(19) United States
(12) Patent Application Publication
(10) Pub. No.: US 2004/0043903 A1
(43) Pub. Date: Mar. 4, 2004

(54) 2-Aryl-5-trifluoromethylpyridines

(76) Inventors: Michael Puhl, Lampertheim (DE); Andreas Gypser, Mannheim (DE); Gerhard Hamprecht, Weinheim (DE); Thorsten Volk, Mannheim (DE); Peter Schafer, Ottersheim (DE); Robert Reinhard, Ludwigshafen (DE); Ingo Sagasser, Dannstadt-Schauernheim (DE); Cyrill Zagar, Mannheim (DE); Matthias Witschel, Bad Durkheim (DE); Andreas Landes, Romerberg (DE)

Correspondence Address: KEIL & WEINKAUF, 1350 CONNECTICUT AVENUE, N.W., WASHINGTON, DC, 20036, US

(21) Appl. No.: 10/416,462

(22) PCT Filed: Nov. 21, 2001
(86) PCT No.: PCT/EP01/13534

(30) Foreign Application Priority Data
Nov. 22, 2000 (DE) 10057854.3

(51) Int. Cl. A01N 43/40; C07D 213/84

(52) U.S. Cl. 504/244; 546/286; 546/329

(57) ABSTRACT

The present invention relates to 2-aryl-5-trifluoromethylpyridines of the formula I

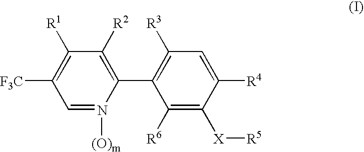

in which the variables m, R¹, R², R³, R⁴, R⁵, R⁶ and X have the meanings given in claim 1, and their agriculturally tolerated salts.

Moreover, the invention relates to the use of compounds I and their salts as herbicides and/or for the desiccation and/or defoliation of plants, to herbicidal compositions and compositions for the desiccation and/or defoliation of plants comprising the compounds I and/or their salts as active substances.

The present invention relates to 2-aryl-5-trifluoromethylpyridines, to their pyridine N-oxides and their agriculturally useful salts, and to their use as herbicides, desiccants or defoliants.

Herbicidally active 2-aryl-5-trifluoromethylpyridines have been described on several occasions in the prior art (see, for example, DE 4323916, WO 95/02580, WO 95/02590, WO 96/21645, WO 96/21646, WO 96/21647, WO 96/21645, WO 97/06143, WO 97/11059, WO 97/30059, WO 98/07700 and WO 99/06394).

The prior-art 2-aryl-5-trifluoromethylpyridines leave something to be desired in some cases with regard to their activity and/or selectivity with respect to harmful plants. Moreover, there is a constant need to provide novel herbicidally active substances to avoid the possibility of resistance build-up against known herbicides.

It was an object of the present invention to provide novel herbicides by means of which harmful plants can be controlled better than hitherto. Advantageously, the novel herbicides should have a high activity with regard to harmful plants. Moreover, crop plant tolerance is desired.

We have found that this object is achieved, surprisingly, by 2-aryl-5-trifluoromethylpyridines, their N-oxides and their agriculturally useful salts which have a particularly high herbicidal activity when they have an amino group or a methyl group in the 4-position of the pyridine ring, a halogen atom being attached in the 3-position and the 6-position being unsubstituted.

Accordingly, the present invention relates to 2-aryl-5-trifluoromethylpyridines of the formula I

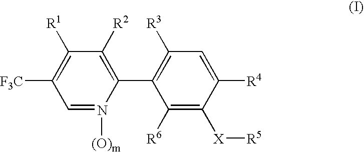

in which the variables m, R¹, R², R³, R⁴, R⁵, R⁶ and X have the following meanings:

m is 0 or 1,

X is a chemical bond, a methylene, 1,2-ethylene, propane-1,3-diyl, ethene-1,2-diyl or ethyne-1,2-diyl chain, or an oxymethylene or thiamethylene chain bonded to the phenyl ring via the hetero atom, it being possible for all chains to be unsubstituted or to have attached to them one or two substituents, in each case selected from the group consisting of cyano, carboxyl, halogen, C₁-C₄-alkyl, C₁-C₄-haloalkyl, C₁-C₄-alkoxy, (C₁-C₄-alkoxy)carbonyl, di(C₁-C₄-alkyl)amino and phenyl;

R¹ is NH₂ or CH₃;

R² is halogen;

R³ is hydrogen or halogen;

R⁴ is halogen, cyano, OH, C₁-C₄-alkoxy or C₁-C₄-alkoxycarbonyl-C₁-C₄-alkoxy;

R⁵ is hydrogen, nitro, cyano, halogen, halosulfonyl, N₃, —O—Y—R⁷, —O—CO—Y—R⁷, —N(Y—R⁷)(Z-R⁸), —N(Y—R⁷)—SO₂-Z-R⁸, —N(SO₂—Y—R⁷)(SO₂-Z-R⁸), —N(Y—R⁷)—CO-Z-R⁸, —N(Y—R⁷)(O-Z-R⁸), —S—Y—R⁷, —SO—Y—R⁷, —SO₂—Y—R⁷, —SO₂—O—Y—R⁷, —SO₂—N(Y—R⁷)(Z-R⁸), —CO—Y—R⁷, —C(═NOR⁹)—Y—R⁷, —C(═NOR⁹)—O—Y—R⁷, —CO—O—Y—R⁷, —CO—S—Y—R⁷, —CO—N(Y—R⁷)(Z-R⁸), —CO—N(Y—R⁷)(O-Z-R⁸) or —PO(O—Y—R⁷)₂;

R⁶ is hydrogen or

R⁴ and X—R⁵ or X—R⁵ and R⁶ are a 3- or 4-membered chain whose chain members, in addition to carbon, can have 1, 2 or 3 hetero atoms selected from among nitrogen, oxygen and sulfur atoms, which hetero atoms can be unsubstituted or can have attached to them, in turn, one, two or three substituents, and whose members can also encompass one or two nonadjacent carbonyl, thiocarbonyl or sulfonyl groups,

Y, Z independently of one another are:

a chemical bond, a methylene or ethylene group which can be unsubstituted or can have attached to it one or two substituents, in each case selected from the group consisting of carboxyl, C₁-C₄-alkyl, C₁-C₄-haloalkyl, (C₁-C₄-alkoxy)carbonyl and phenyl;

R⁷, R⁸ independently of one another are:

hydrogen, C₃-C₈-cycloalkyl-C₁-C₄-alkyl, C₁-C₆-alkyl, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, —CH(R¹⁰) (R¹¹), —C(R¹⁰) (R¹¹)—CN, —C(R¹⁰) (R¹¹)-halogen, —C(R¹⁰)(R¹¹)—OR¹², —C(R¹⁰)(R¹¹)—N(R¹²)R¹³, —C(R¹⁰)(R¹¹)—N(R¹²)—OR¹³, —C(R¹⁰)(R¹¹)—SR¹², —C(R¹⁰)(R¹¹)—SO—R¹², —C(R¹⁰) (R¹¹)—SO₂—R¹², —C(R¹⁰) (R¹¹)—SO₂—OR¹², —C(R¹⁰)(R¹¹)—SO₂—N(R¹²)R¹³, —C(R¹⁰)(R¹¹)—CO—R¹², —C(R¹⁰)(R¹¹)—C(═NOR¹⁴)—R¹², —C(R¹⁰)(R¹¹)—CO—OR¹², —C(R¹⁰)(R¹¹)—CO—SR¹², —C(R¹⁰)(R¹¹)—CO—N(R¹²)R¹³, —C(R¹⁰)(R¹¹)—CO—N(R¹²)—OR¹³, —C(R¹⁰)(R¹¹)—PO(OR¹²)₂, C₃-C₈-cycloalkyl which can contain a carbonyl or thiocarbonyl ring member,

phenyl or 3-, 4-, 5-, 6- or 7-membered heterocyclyl which can contain a carbonyl or thiocarbonyl ring member,

it being possible for each cycloalkyl ring, for the phenyl ring and for each heterocyclyl ring to be unsubstituted or to have attached to it one, two, three or four substituents, in each case selected from the group consisting of cyano, nitro, amino, hydroxyl, carboxyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, (C₁-C₄-alkoxy)carbonyl and di(C₁-C₄-alkyl)amino;

R⁹ is hydrogen, C₁-C₆-alkyl, C₁-C₄-alkoxycarbonyl-C₁-C₄-alkoxy, C₄-C₈-cycloalkyl-C₁-C₄-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;

where the variables R¹⁰ to R¹⁴ have the following meanings:

R¹⁰, R¹¹ independently of one another are

hydrogen, C₁-C₄-alkyl, C₁-C₄-alkoxy-C₁-C₄-alkyl, C₁-C₄-alkylthio-C₁-C₄-alkyl, (C₁-C₄-alkoxy)carbonyl-C₁-C₄-alkyl or phenyl-C₁-C₄-alkyl, it being possible for the phenyl ring to be unsubstituted or to have attached to it one to three substituents, in each case selected from the group consisting of cyano, nitro, carboxyl, halogen, C₁-C₄-alkyl, C₁-C₄-haloalkyl and (C₁-C₄-alkoxy)carbonyl;

R¹², R¹³ independently of one another are

hydrogen, C₁-C₆-alkyl, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, C₃-C₈-cycloalkyl, C₃-C₈-cycloalkyl-C₁-C₄-alkyl, phenyl, phenyl-C₁-C₄-alkyl, 3- to 7-membered heterocyclyl or heterocyclyl-C₁-C₄-alkyl, it being possible for each cycloalkyl and each heterocyclyl ring to contain a carbonyl or thiocarbonyl ring member, and where each cycloalkyl ring, the phenyl ring and each heterocyclyl ring can be unsubstituted or have attached to it one, two, three or four substituents, in each case selected from the group consisting of cyano, nitro, amino, hydroxyl, carboxyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, (C₁-C₄-alkoxy)carbonyl and di(C₁-C₄-alkyl)amino;

R¹⁴ is hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₁-C₄-alkoxycarbonyl-C₁-C₄-alkyl, phenyl or phenyl-C₁-C₄-alkyl;

and the agriculturally useful salts of I.

Furthermore, the invention relates to:

the use of compounds I and their salts as herbicides and/or for the desiccation and/or defoliation of plants,

herbicidal compositions and compositions for the desiccation and/or defoliation of plants comprising the compounds I and/or their salts as active substances,

intermediates for the preparation of the compounds I

processes for the preparation of herbicidal compositions and compositions for the desiccation and/or defoliation of plants using the compounds I, and

methods of controlling undesired vegetation (harmful plants) and for the desiccation and/or defoliation of plants with the compounds I and/or their salts.

The compounds of the formula I can form geometric isomers, for example E/Z isomers, in the substituents. The invention relates not only to the pure isomers, but also to their mixtures. Moreover, the compounds of the formula I can have one or more chiral centers in the substituents, in which case they are present as enantiomer or diastereomer mixtures. The invention relates to the pure enantiomers and diastereomers and also to their mixtures.

Suitable among agriculturally useful salts are especially the salts of those cations and the acid addition salts of those acids whose cations, or anions, do not adversely affect the herbicidal action of the compounds I. Thus, suitable cations are, in particular, the ions of the alkali metals, preferably sodium and potassium, of the alkaline earth metals, preferably calcium, magnesium and barium, and of the transition metals, preferably manganese, copper, zinc and iron, and the ammonium ion which, if desired, can have attached to it one to four C₁-C₄-alkyl substituents and/or a phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium and sulfoxonium ions, preferably tri(C₁-C₄-alkyl)sulfoxonium.

Anions of useful acid addition salts are mainly chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting I with an acid of the anion in question, preferably hydrochloric acid, hydrobromic acid, sulfuric acid, phosphoric acid or nitric acid.

The organic moieties mentioned in the definition of the substituents R¹, R², R⁴, R⁷ to R¹⁸ or as radicals on cycloalkyl rings, phenyl rings or heterocyclic rings or on X, Y and Z constitute, like the meaning halogen, collective terms for individual enumerations of the individual group members. All carbon chains, i.e. all alkyl, haloalkyl, phenylalkyl, cycloalkylalkyl, alkoxy, haloalkoxy, alkylthio, haloalkylthio, alkylsulfinyl, haloalkylsulfinyl, alkylsulfonyl, haloalkylsulfonyl, alkenyl, haloalkenyl, alkynyl and haloalkynyl groups and corresponding moieties in larger groups such as alkoxycarbonyl, phenylalkyl, cycloalkylalkyl, alkoxycarbonylalkyl etc. can be straight-chain or branched, the prefix Cₙ-Cₘ in each case indicating the possible number of carbon atoms in the group. Halogenated substituents preferably have attached to them one, two, three, four or five identical or different halogen atoms. The meaning halogen denotes in each case fluorine, chlorine, bromine or iodine.

Other examples of meanings are:

—C₁-C₄-alkyl: CH₃, C₂H₅, n-propyl, CH(CH₃)₂, n-butyl, CH(CH₃)—C₂H₅, CH₂—CH(CH₃)₂ and C(CH₃)₃;

—C₁-C₄-haloalkyl: a C₁-C₄-alkyl radical as mentioned above which is partially or fully substituted by fluorine, chlorine, bromine and/or iodine, i.e., for example, CH₂F, CHF₂, CF₃, CH₂Cl, dichloromethyl, trichloromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, C₂F₅, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl;

—C₁-C₆-alkyl: C₁-C₄-alkyl as mentioned above and also, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably methyl, ethyl, n-propyl, 1-methylethyl, n-butyl, 1,1-dimethylethyl, n-pentyl or n-hexyl;

C₁-C₆-haloalkyl: a C₁-C₆-alkyl radical as mentioned above which is partially or fully substituted by fluorine, chlorine, bromine and/or iodine, i.e., for example, one of the radicals mentioned under C₁-C₄-haloalkyl and also 5-fluoro-1-pentyl, 5-chloro-1-pentyl, 5-bromo-1-pentyl, 5-iodo-1-pentyl, 5,5,5-trichloro-1-pentyl, undecafluoropentyl, 6-fluoro-1-hexyl, 6-chloro-1-hexyl, 6-bromo-1-hexyl, 6-iodo-1-hexyl, 6,6,6-trichloro-1-hexyl or dodecafluorohexyl;

phenyl-C₁-C₄-alkyl: benzyl, 1-phenylethyl, 2-phenylethyl, 1-phenylprop-1-yl, 2-phenylprop-1-yl, 3-phenylprop-1-yl, 1-phenylbut-1-yl, 2-phenylbut-1-yl, 3-phenylbut-1-yl, 4-phenylbut-1-yl, 1-phenylbut-2-yl, 2-phenylbut-2-yl, 3-phenylbut-2-yl, 4-phenylbut-2-yl, 1-(phenylmethyl)eth-1-yl, 1-(phenylmethyl)-1-(methyl)eth-1-yl or 1-(phenylmethyl)prop-1-yl, preferably benzyl or 2-phenylethyl;

heterocyclyl-C₁-C₄-alkyl: heterocyclylmethyl, 1-heterocyclylethyl, 2-heterocyclylethyl, 1-heterocyclylprop-1-yl, 2-heterocyclylprop-1-yl, 3-heterocyclylprop-1-yl, 1-heterocyclylbut-1-yl, 2-heterocyclylbut-1-yl, 3-heterocyclylbut-1-yl, 4-heterocyclylbut-1-yl, 1-heterocyclylbut-2-yl, 2-heterocyclylbut-2-yl, 3-heterocyclylbut-2-yl, 4-heterocyclylbut-2-yl, 1-(heterocyclylmethyl)eth-1-yl, 1-(heterocyclylmethyl)-1-(methyl)eth-1-yl or 1-(heterocyclylmethyl)prop-1-yl, preferably heterocyclylmethyl or 2-heterocyclylethyl;

C₁-C₄-alkoxy: OCH₃, OC₂H₅, n-propoxy, OCH(CH₃)₂, n-butoxy, OCH(CH₃)—C₂H₅, OCH₂—CH(CH₃)₂ or OC(CH₃)₃, preferably OCH₃, OC₂H₅ or OCH(CH₃)₂;

C₁-C₄-haloalkoxy: a C₁-C₄-alkoxy radical as mentioned above which is partially or fully substituted by fluorine, chlorine, bromine and/or iodine, i.e., for example, OCH₂F, OCHF₂, OCF₃, OCH₂Cl, OCH(Cl)₂, OC(Cl)₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, OC₂F₅, 2-fluoropropoxy, 3-fluoropropoxy, 2,2-difluoropropoxy, 2,3-difluoropropoxy, 2-chloropropoxy, 3-chloropropoxy, 2,3-dichloropropoxy., 2-bromopropoxy, 3-bromopropoxy, 3,3,3-trifluoropropoxy, 3,3,3-trichloropropoxy, 2,2,3,3,3-pentafluoropropoxy, OCF₂—C₂F₅, 1-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy, preferably OCHF₂, OCF₃, dichlorofluoromethoxy, chlorodifluoromethoxy or 2,2,2-trifluoroethoxy;

C₁-C₄-alkylthio: SCH₃, SC₂Hs, n-propylthio, SCH(CH₃)₂, n-butylthio, SCH(CH₃)—C₂H₅, SCH₂—CH(CH₃)₂ or SC(CH₃)₃, preferably SCH₃ or SC₂H₅;

—C₁-C₄-haloalkylthio: a C₁-C₄-alkylthio radical as mentioned above which is partially or fully substituted by fluorine, chlorine, bromine and/or iodine, i.e., for example, SCH₂F, SCHF₂, SCH₂Cl, SCH(Cl)₂, SC(Cl)₃, SCF₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio, SC₂F₅, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or SCF₂—CF₂—C₂F₅, preferably SCHF₂, SCF₃, dichlorofluoromethylthio, chlorodifluoromethylthio or 2,2,2-trifluoroethylthio;

C₁-C₄-alkoxy-C₁-C₄-alkyl: C₁-C₄-alkyl which is substituted by C₁-C₄-alkoxy as mentioned above, i.e., for example, CH₂—OCH₃, CH₂—OC₂H₅, n-propoxymethyl, CH₂—OCH(CH₃)₂, n-butoxymethyl, (1-methylpropoxy)methyl, (2-methylpropoxy)methyl, CH₂—OC(CH₃)₃, 2-(methoxy)ethyl, 2-(ethoxy)ethyl, 2-(n-propoxy)ethyl, 2-(1-methylethoxy)ethyl, 2-(n-butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy)propyl, 2-(n-propoxy)propyl, 2-(1-methylethoxy)propyl, 2-(n-butoxy)propyl, 2-(1-methylpropoxy)propyl, 2-(2-methylpropoxy)propyl, 2-(1,1-dimethylethoxy)propyl, 3-(methoxy)propyl, 3-(ethoxy)propyl, 3-(n-propoxy)propyl, 3-(1-methylethoxy)propyl, 3-(n-butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(n-propoxy)butyl, 2-(1-methylethoxy)butyl, 2-(n-butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(n-propoxy)butyl, 3-(1-methylethoxy)butyl, 3-(n-butoxy)butyl, 3-(1-methylpropoxy)butyl, 3-(2-methylpropoxy)butyl, 3-(1,1-dimethylethoxy)butyl, 4-(methoxy)butyl, 4-(ethoxy)butyl, 4-(n-propoxy)butyl, 4-(1-methylethoxy)butyl, 4-(n-butoxy)butyl, 4-(1-methylpropoxy)butyl, 4-(2-methylpropoxy)butyl or 4-(1,1-dimethylethoxy)butyl, preferably CH₂—OCH₃, CH₂—OC₂H₅, 2-methoxyethyl or 2-ethoxyethyl;

C₁-C₄-alkylthio-C₁-C₄-alkyl: C₁-C₄-alkyl which is substituted by C₁-C₄-alkylthio as mentioned above, i.e., for example, CH₂—SCH₃, CH₂—SC₂H₅, n-propylthiomethyl, CH₂—SCH(CH₃)₂, n-butylthiomethyl, (1-methylpropylthio)methyl, (2-methylpropylthio)methyl, CH₂—SC(CH₃)₂, 2-(methylthio)ethyl, 2-(ethylthio)ethyl, 2-(n-propylthio)ethyl, 2-(1-methylethylthio)ethyl, 2-(n-butylthio)ethyl, 2-(1-methylpropylthio)ethyl, 2-(2-methylpropylthio)ethyl, 2-(1,1-dimethylethylthio)ethyl, 2-(methylthio)propyl, 2-(ethylthio)propyl, 2-(n-propylthio)propyl, 2-(1-methylethylthio)propyl, 2-(n-butylthio)propyl, 2-(1-methylpropylthio)propyl, 2-(2-methylpropylthio)propyl, 2-(1,1-dimethylethylthio)propyl, 3-(methylthio)propyl, 3-(ethylthio)propyl, 3-(n-propylthio)propyl, 3-(1-methylethylthio)propyl, 3-(n-butylthio)propyl, 3-(1-methylpropylthio)propyl, 3-(2-methylpropylthio)propyl, 3-(1,1-dimethylethylthio)propyl, 2-(methylthio)butyl, 2-(ethylthio)butyl, 2-(n-propylthio)butyl, 2-(1-methylethylthio)butyl, 2-(n-butylthio)butyl, 2-(1-methylpropylthio)butyl, 2-(2-methylpropylthio)butyl, 2-(1,1-dimethylethylthio)butyl, 3-(methylthio)butyl, 3-(ethylthio)butyl, 3-(n-propylthio)butyl, 3-(1-methylethylthio)butyl, 3-(n-butylthio)butyl, 3-(1-methylpropylthio)butyl, 3-(2-methylpropylthio)butyl, 3-(1,1-dimethylethylthio)butyl, 4-(methylthio)butyl, 4-(ethylthio)butyl, 4-(n-propylthio)butyl, 4-(1-methylethylthio)butyl, 4-(n-butylthio)butyl, 4-(1-methylpropylthio)butyl, 4-(2-methylpropylthio)butyl or 4-(1,1-dimethylethylthio)butyl, preferably CH₂—SCH₃, CH₂—SC₂H₅, 2-methylthioethyl or 2-ethylthioethyl;

(C₁-C₄-alkyl)carbonyl: CO—CH₃, CO—C₂H₅, CO—CH₂—C₂H₅, CO—CH(CH₃)₂, n-butylcarbonyl, CO—CH(CH₃)—C₂H₅, CO—CH₂—CH(CH₃)₂ or CO—C(CH₃)₃, preferably CO—CH₃ or CO—C₂H₅;

(C₁-C₄-haloalkyl)carbonyl: a (C₁-C₄-alkyl)carbonyl radical as mentioned above which is partially or fully substituted by fluorine, chlorine, bromine and/or iodine, i.e., for example, CO—CH₂F, CO—CHF₂, CO—CF₃, CO—CH₂C₁, CO—CH(Cl)₂, CO—C(Cl)₃, chlorofluoromethylcarbonyl, dichlorofluoromethylcarbonyl, chlorodifluoromethylcarbonyl, 2-fluoroethylcarbonyl, 2-chloroethylcarbonyl, 2-bromoethylcarbonyl, 2-iodoethylcarbonyl, 2,2-difluoroethylcarbonyl, 2,2,2-trifluoroethylcarbonyl, 2-chloro-2-fluoroethylcarbonyl, 2-chloro-2,2-difluoroethylcarbonyl, 2,2-dichloro-2-fluoroethylcarbonyl, 2,2,2-trichloroethylcarbonyl, CO—C₂F₅, 2-fluoropropylcarbonyl, 3-fluoropropylcarbonyl, 2,2-difluoropropylcarbonyl, 2,3-difluoropropylcarbonyl, 2-chloropropylcarbonyl, 3-chloropropylcarbonyl, 2,3-dichloropropylcarbonyl, 2-bromopropylcarbonyl, 3-bromopropylcarbonyl, 3,3,3-trifluoropropylcarbonyl, 3,3,3-trichloropropylcarbonyl, 2,2,3,3,3-pentafluoropropylcarbonyl, CO—CF₂—C₂F₅, 1-(CH₂F)-2-fluoroethylcarbonyl, 1-(CH₂Cl)-2-chlorothylcarbonyl, 1-(CH₂Br)-2-bromoethylcarbonyl, 4-fluorobutylcarbonyl, 4-chlorobutylcarbonyl, 4-bromobutylcarbonyl or nonafluorobutylcarbonyl, preferably CO—CF₃, CO—CH₂C₁, or 2,2,2-trifluoroethylcarbonyl;

(C₁-C₄-alkyl)carbonyloxy: O—CO—CH₃, O—CO—C₂H₅, O—CO—CH₂—C₂H₅, O—CO—CH(CH₃)₂, O—CO—CH₂—CH₂—C₂H₅, O—CO—CH(CH₃)—C₂H₅, O—CO—CH₂—CH(CH₃)₂ or O—CO—C(CH₃)₃, preferably O—CO—CH₃ or O—CO—C₂H₅;

(C₁-C₄-haloalkyl)carbonyloxy: a (C₁-C₄-alkyl)carbonyl radical as mentioned above which is partially or fully substituted by fluorine, chlorine, bromine and/or iodine, i.e., for example, O—CO—CH₂F, O—CO—CHF₂, O—CO—CF₃, O—CO—CH₂Cl, O—CO—CH(Cl)₂, —CO—C(Cl)₃, chlorofluoromethylcarbonyloxy, dichlorofluoromethylcarbonyloxy, chlorodifluoromethylcarbonyloxy, 2-fluoroethylcarbonyloxy, 2-chloroethylcarbonyloxy, 2-bromoethylcarbonyloxy, 2-iodoethylcarbonyloxy, 2,2-difluoroethylcarbonyloxy, 2,2,2-trifluoroethylcarbonyloxy, 2-chloro-2-fluoroethylcarbonyloxy, 2-chloro-2,2-difluoroethylcarbonyloxy, 2,2-dichloro-2-fluoroethylcarbonyloxy, 2,2,2-trichloroethylcarbonyloxy, O—CO—C₂F₅, 2-fluoropropylcarbonyloxy, 3-fluoropropylcarbonyloxy, 2,2-difluoropropylcarbonyloxy, 2,3-difluoropropylcarbonyloxy, 2-chloropropylcarbonyloxy, 3-chloropropylcarbonyloxy, 2,3-dichloropropylcarbonyloxy, 2-bromopropylcarbonyloxy, 3-bromopropylcarbonyloxy, 3,3,3-trifluoropropylcarbonyloxy, 3,3,3-trichloropropylcarbonyloxy, 2,2,3,3,3-pentafluoropropylcarbonyloxy, heptafluoropropylcarbonyloxy, 1-(CH₂F)-2-fluoroethylcarbonyloxy, 1-(CH₂Cl)-2-chlorothylcarbonyloxy, 1-(CH₂Br)-2-bromoethylcarbonyloxy, 4-fluorobutylcarbonyloxy, 4-chlorobutylcarbonyloxy, 4-bromobutylcarbonyloxy or nonafluorobutylcarbonyloxy, preferably O—CO—CF₃, O—CO—CH₂Cl or 2,2,2-trifluoroethylcarbonyloxy;

(C₁-C₄-alkoxy)carbonyl: CO—OCH₃, CO—OC₂H₅, n-propoxycarbonyl, CO—OCH(CH₃)₂, n-butoxycarbonyl, CO—OCH(CH₃)—C₂H₅, CO—OCH₂—CH(CH₃)₂ or CO—OC(CH₃)₃, preferably CO—OCH₃ or CO—OC₂H₅;

(C₁-C₄-alkoxy)carbonyl-C₁-C₄-alkyl: C₁-C₄-alkyl which is substituted by (C₁-C₄-alkoxy)carbonyl as mentioned above, i.e., for example, methoxycarbonylmethyl, ethoxycarbonylmethyl, n-propoxycarbonylmethyl, (1-methylethoxycarbonyl)methyl, n-butoxycarbonylmethyl, (1-methylpropoxycarbonyl)methyl, (2-methylpropoxycarbonyl)methyl, (1,1-dimethylethoxycarbonyl)methyl, 1-(methoxycarbonyl)ethyl, 1-(ethoxycarbonyl)ethyl, 1-(n-propoxycarbonyl)ethyl, 1-(1-methylethoxycarbonyl)ethyl, 1-(n-butoxycarbonyl)ethyl, 2-(methoxycarbonyl)ethyl, 2-(ethoxycarbonyl)ethyl, 2-(n-propoxycarbonyl)ethyl, 2-(1-methylethoxycarbonyl)ethyl, 2-(n-butoxycarbonyl)ethyl, 2-(1-methylpropoxycarbonyl)ethyl, 2-(2-methylpropoxycarbonyl)ethyl, 2-(1,1-dimethylethoxycarbonyl)ethyl, 1-(methoxycarbonyl)-1-methylethyl, 1-(ethoxycarbonyl)-1-methylethyl, 1-(n-propoxycarbonyl)-1-methylethyl, 1-(1-methylethoxycarbonyl)-1-methylethyl, 1-(n-butoxycarbonyl)-1-methylethyl, 2-(methoxycarbonyl)propyl, 2-(ethoxycarbonyl)propyl, 2-(n-propoxycarbonyl)propyl, 2-(1-methylethoxycarbonyl)propyl, 2-(n-butoxycarbonyl)propyl, 2-(1-methylpropoxycarbonyl)propyl, 2-(2-methylpropoxycarbonyl)propyl, 2-(1,1-dimethylethoxycarbonyl)propyl, 3-(methoxycarbonyl)propyl, 3-(ethoxycarbonyl)propyl, 3-(n-propoxycarbonyl)propyl, 3-(1-methylethoxycarbonyl)propyl, 3-(n-butoxycarbonyl)propyl, 3-(1-methylpropoxycarbonyl)propyl, 3-(2-methylpropoxycarbonyl)propyl, 3-(1,1-dimethylethoxycarbonyl)propyl, 2-(methoxycarbonyl)-butyl, 2-(ethoxycarbonyl)butyl, 2-(n-propoxycarbonyl)butyl, 2-(1-methylethoxycarbonyl)butyl, 2-(n-butoxycarbonyl)butyl, 2-(1-methylpropoxycarbonyl)butyl, 2-(2-methylpropoxycarbonyl)butyl, 2-(1,1-dimethylethoxycarbonyl)butyl, 3-(methoxycarbonyl)butyl, 3-(ethoxycarbonyl)butyl, 3-(n-propoxycarbonyl)butyl, 3-(1-methylethoxycarbonyl)butyl, 3-(n-butoxycarbonyl)butyl, 3-(1-methylpropoxycarbonyl)butyl, 3-(2-methylpropoxycarbonyl)butyl, 3-(1,1-dimethylethoxycarbonyl)butyl, 4-(methoxycarbonyl)butyl, 4-(ethoxycarbonyl)butyl, 4-(n-propoxycarbonyl)butyl, 4-(1-methylethoxycarbonyl)butyl, 4-(n-butoxycarbonyl)butyl, 4-(1-methylpropoxycarbonyl)butyl, 4-(2-methylpropoxycarbonyl)butyl or 4-(1,1-dimethylethoxycarbonyl)butyl, preferably methoxycarbonylmethyl, ethoxycarbonylmethyl, 1-(methoxycarbonyl)ethyl or 1-(ethoxycarbonyl )ethyl;

(C₁-C₄-alkoxy)carbonyl-C₁-C₄-alkoxy: C₁-C₄-alkoxy which is substituted by (C₁-C₄-alkoxy)carbonyl as mentioned above, i.e., for example, methoxycarbonylmethoxy, ethoxycarbonylmethoxy, n-propoxycarbonylmethoxy, (1-methylethoxycarbonyl)methoxy, n-butoxycarbonylmethoxy, (1-methylpropoxycarbonyl)methoxy, (2-methylpropoxycarbonyl)methoxy, (1,1-dimethylethoxycarbonyl)methoxy, 1-(methoxycarbonyl)ethoxy, 1-(ethoxycarbonyl)ethoxy, 1-(n-propoxycarbonyl)ethoxy, 1-(1-methylethoxycarbonyl)ethoxy, 1-(n-butoxycarbonyl)ethoxy, 2-(methoxycarbonyl)ethoxy, 2-(ethoxycarbonyl)ethoxy, 2-(n-propoxycarbonyl)ethoxy, 2-(1-methylethoxycarbonyl)ethoxy, 2-(n-butoxycarbonyl)ethoxy, 2-(1-methylpropoxycarbonyl)ethoxy, 2-(2-methylpropoxycarbonyl)ethoxy, 2-(1,1-dimethylethoxycarbonyl)ethoxy, 1-(methoxycarbonyl)-1-methylethoxy, 1-(ethoxycarbonyl)-1-methylethoxy, 1-(n-propoxycarbonyl)-1-methylethoxy, 1-(1-methylethoxycarbonyl)-1-methylethoxy, 1-(n-butoxycarbonyl)-1-methylethoxy, 2-(methoxycarbonyl)propoxy, 2-(ethoxycarbonyl)propoxy, 2-(n-propoxycarbonyl)propoxy, 2-(1-methylethoxycarbonyl)propoxy, 2-(n-butoxycarbonyl)propoxy, 2-(1-methylpropoxycarbonyl)propoxy, 2-(2-methylpropoxycarbonyl)propoxy, 2-(1,1-dimethylethoxycarbonyl)propoxy, 3-(methoxycarbonyl)propoxy, 3-(ethoxycarbonyl)propoxy, 3-(n-propoxycarbonyl)propoxy, 3-(1-methylethoxycarbonyl)propoxy, 3-(n-butoxycarbonyl)propoxy, 3-(1-methylpropoxycarbonyl)propoxy, 3-(2-methylpropoxycarbonyl)propoxy, 3-(1,1-dimethylethoxycarbonyl)propoxy, 2-(methoxycarbonyl)butoxy, 2-(ethoxycarbonyl)butoxy, 2-(n-propoxycarbonyl)butoxy, 2-(1-methylethoxycarbonyl)butoxy, 2-(n-butoxycarbonyl)butoxy, 2-(1-methylpropoxycarbonyl)butoxy, 2-(2-methylpropoxycarbonyl)butoxy, 2-(1,1-dimethylethoxycarbonyl)butoxy, 3-(methoxycarbonyl)butoxy, 3-(ethoxycarbonyl)butoxy, 3-(n-propoxycarbonyl)butoxy, 3-(1-methylethoxycarbonyl)butoxy, 3-(n-butoxycarbonyl)butoxy, 3-(1-methylpropoxycarbonyl)butoxy, 3-(2-methylpropoxycarbonyl)butoxy, 3-(1,1-dimethylethoxycarbonyl)butoxy, 4-(methoxycarbonyl)butoxy, 4-(ethoxycarbonyl)butoxy, 4-(n-propoxycarbonyl)butoxy, 4-(1-methylethoxycarbonyl)butoxy, 4-(n-butoxycarbonyl)butoxy, 4-(1-methylpropoxycarbonyl)butoxy, 4-(2-methylpropoxycarbonyl)butyl or 4-(1,1-dimethylethoxycarbonyl)butoxy, preferably methoxycarbonylmethoxy, ethoxycarbonylmethoxy, 1-(methoxycarbonyl)ethoxy or 1-(ethoxycarbonyl)ethoxy;

(C₁-C₄-alkoxy)carbonyl-C₁-C₄-alkylthio: C₁-C₄-alkylthio which is substituted by (C₁-C₄-alkoxy)carbonyl as mentioned above, i.e., for example, methoxycarbonylmethylthio, ethoxycarbonylmethylthio, n-propoxycarbonylmethylthio, (1-methylethoxycarbonyl)methylthio, n-butoxycarbonylmethylthio, (1-methylpropoxycarbonyl)methylthio, (2-methylpropoxycarbonyl)methylthio, (1,1-dimethylethoxycarbonyl)methylthio, 1-(methoxycarbonyl)ethylthio, 1-(ethoxycarbonyl)ethylthio, 1-(n-propoxycarbonyl)ethylthio, 1-(1-methylethoxycarbonyl)ethylthio, 1-(n-butoxycarbonyl)ethylthio, 2-(methoxycarbonyl)ethylthio, 2-(ethoxycarbonyl)ethylthio, 2-(n-propoxycarbonyl)ethylthio, 2-(1-methylethoxycarbonyl)ethylthio, 2-(n-butoxycarbonyl)ethylthio, 2-(1-methylpropoxycarbonyl)ethylthio, 2-(2-methylpropoxycarbonyl)ethylthio, 2-(1,1-dimethylethoxycarbonyl)ethylthio, 2-(methoxycarbonyl)propylthio, 2-(ethoxycarbonyl)propylthio, 2-(n-propoxycarbonyl)propylthio, 2-(1-methylethoxycarbonyl)propylthio, 2-(n-butoxycarbonyl)propylthio, 2-(1-methylpropoxycarbonyl)propylthio, 2-(2-methylpropoxycarbonyl)propylthio, 2-(1,1-dimethylethoxycarbonyl)propylthio, 3-(methoxycarbonyl)propylthio, 3-(ethoxycarbonyl)propylthio, 3-(n-propoxycarbonyl)propylthio, 3-(1-methylethoxycarbonyl)propylthio, 3-(n-butoxycarbonyl)propylthio, 3-(1-methylpropoxycarbonyl)propylthio, 3-(2-methylpropoxycarbonyl)propylthio, 3-(1,1-dimethylethoxycarbonyl)propylthio, 2-(methoxycarbonyl)butylthio, 2-(ethoxycarbonyl)butylthio, 2-(n-propoxycarbonyl)butylthio, 2-(1-methylethoxycarbonyl)butylthio, 2-(n-butoxycarbonyl)butylthio, 2-(1-methylpropoxycarbonyl)butylthio, 2-(2-methylpropoxycarbonyl)butylthio, 2-(1,1-dimethylethoxycarbonyl)butylthio, 3-(methoxycarbonyl)butylthio, 3-(ethoxycarbonyl)butylthio, 3-(n-propoxycarbonyl)butylthio, 3-(1-methylethoxycarbonyl)butylthio, 3-(n-butoxycarbonyl)butylthio, 3-(1-methylpropoxycarbonyl)butylthio, 3-(2-methylpropoxycarbonyl)butylthio, 3-(1,1-dimethylethoxycarbonyl)butylthio, 4-(methoxycarbonyl)butylthio, 4-(ethoxycarbonyl)butylthio, 4-(n-propoxycarbonyl)butylthio, 4-(1-methylethoxycarbonyl)butylthio, 4-(n-butoxycarbonyl)butylthio, 4-(1-methylpropoxycarbonyl)butylthio, 4-(2-methylpropoxycarbonyl)butyl or 4-(1,1-dimethylethoxycarbonyl)butylthio, preferably methoxycarbonylmethylthio, ethoxycarbonylmethylthio, 1-(methoxycarbonyl)ethylthio or 1-(ethoxycarbonyl)ethylthio;

C₁-C₄-alkylsulfinyl: SO—CH₃, SO—C₂H₅, SO—CH₂—C₂H₅, SO—CH(CH₃)₂, n-butylsulfinyl, SO—CH(CH₃)—C₂H₅, SO—CH₂—CH(CH₃)₂ or SO—C(CH₃)₃, preferably SO—CH₃ or SO—C₂H₅;

C₁-C₄-haloalkylsulfinyl: a C₁-C₄-alkylsulfinyl radical as mentioned above which is partially or fully substituted by fluorine, chlorine, bromine and/or iodine, i.e., for example, SO—CH₂F, SO—CHF₂, SO—CF₃, SO—CH₂C₁, SO—CH(Cl)₂, SO—C(Cl)₃, chlorofluoromethylsulfinyl, dichlorofluoromethylsulfinyl, chlorodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2-chloro-2-fluoroethylsulfinyl, 2-chloro-2,2-difluoroethylsulfinyl, 2,2-dichloro-2-fluoroethylsulfinyl, 2,2,2-trichloroethylsulfinyl, SO—C₂F₅, 2-fluoropropylsulfinyl, 3-fluoropropylsulfinyl, 2,2-difluoropropylsulfinyl, 2,3-difluoropropylsulfinyl, 2-chloropropylsulfinyl, 3-chloropropylsulfinyl, 2,3-dichloropropylsulfinyl, 2-bromopropylsulfinyl, 3-bromopropylsulfinyl, 3,3,3-trifluoropropylsulfinyl, 3,3,3-trichloropropylsulfinyl, SO—CH₂—C₂F₅, SO—CF₂—C₂F₅, 1-(fluoromethyl)-2-fluoroethylsulfinyl, 1-(chloromethyl)-2-chlorothylsulfinyl, 1-(bromomethyl)-2-bromoethylsulfinyl, 4-fluorobutylsulfinyl, 4-chlorobutylsulfinyl, 4-bromobutylsulfinyl or nonafluorobutylsulfinyl, preferably SO—CF₃, SO—CH₂Cl or 2,2,2-trifluoroethylsulfinyl;

C₁-C₄-alkylsulfonyl: SO₂—CH₃, SO₂—C₂H₅, SO₂—CH₂—C₂H₅, SO₂—CH(CH₃)₂, n-butylsulfonyl, SO₂—CH(CH₃)—C₂H₅, SO₂—CH₂—CH(CH₃)₂ or SO₂—C(CH₃)₃, preferably SO₂—CH₃ or SO₂—C₂H₅;

C₁-C₄-haloalkylsulfonyl: a C₁-C₄-alkylsulfonyl radical as mentioned above which is partially or fully substituted by fluorine, chlorine, bromine and/or iodine, i.e., for example, SO₂—CH₂F, SO₂—CHF₂, SO₂—CF₃, SO₂—CH₂C1, SO₂—CH(Cl)₂, SO₂—C(Cl)₃, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl, SO₂—C₂F₅, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, SO₂—CH₂—C₂F₅, SO₂—CF₂—C₂F₅, 1-(fluoromethyl)-2-fluoroethylsulfonyl, 1-(chloromethyl)-2-chlorothylsulfonyl, 1-(bromomethyl)-2-bromoethylsulfonyl, 4-fluorobutylsulfonyl, 4-chlorobutylsulfonyl, 4-bromobutylsulfonyl or nonafluorobutylsulfonyl, preferably SO₂—CF₃, SO₂—CH₂Cl or 2,2,2-trifluoroethylsulfonyl;

di(C₁-C₄-alkyl)amino: N(CH₃)₂, N(C₂H₅)₂, N,N-dipropylamino, N[CH(CH₃)₂]₂, N,N-dibutylamino, N,N-di(1-methylpropyl)amino, N,N-di(2-methylpropyl)amino, N[C(CH₃)₃]₂, N-ethyl-N-methylamino, N-methyl-N-propylamino, N-methyl-N-(1-methylethyl)amino, N-butyl-N-methylamino, N-methyl-N-(1-methylpropyl)amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methylethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methylpropyl)-N-(2-methylpropyl)amino, N-(1,1-dimethylethyl)-N-(1-methylpropyl)amino or N-(1,1-dimethylethyl)-N-(2-methylpropyl)amino, preferably N(CH₃)₂ or N(C₂H₅);

di(C₁-C₄-alkyl)aminocarbonyl: e.g. N,N-dimethylaminocarbonyl, N,N-diethylaminocarbonyl, N,N-di(1-methylethyl)aminocarbonyl, N,N-dipropylaminocarbonyl, N,N-dibutylaminocarbonyl, N,N-di(1-methylpropyl)aminocarbonyl, N,N-di(2-methylpropyl)aminocarbonyl, N,N-di(1,1-dimethylethyl)aminocarbonyl, N-ethyl-N-methylaminocarbonyl, N-methyl-N-propylaminocarbonyl, N-methyl-N-(1-methylethyl)aminocarbonyl, N-butyl-N-methylaminocarbonyl, N-methyl-N-(1-methylpropyl)aminocarbonyl, N-methyl-N-(2-methylpropyl)aminocarbonyl, N-(1,1-dimethylethyl)-N-methylaminocarbonyl, N-ethyl-N-propylaminocarbonyl, N-ethyl-N-(1-methylethyl)aminocarbonyl, N-butyl-N-ethylaminocarbonyl, N-ethyl-N-(1-methylpropyl)aminocarbonyl, N-ethyl-N-(2-methylpropyl)aminocarbonyl, N-ethyl-N-(1,1-dimethylethyl)aminocarbonyl, N-(1-methylethyl)-N-propylaminocarbonyl, N-Butyl-N-propylaminocarbonyl, N-(1-methylpropyl)-N-propylaminocarbonyl, N-(2-methylpropyl)-N-propylaminocarbonyl, N-(1,1-dimethylethyl)-N-propylaminocarbonyl, N-butyl-N-(1-methylethyl)aminocarbonyl, N-(1-methylethyl)-N-(1-methylpropyl)aminocarbonyl, N-(1-methylethyl)-N-(2-methylpropyl)aminocarbonyl, N-(1,1-dimethylethyl)-N-(1-methylethyl)aminocarbonyl, N-butyl-N-(1-methylpropyl)aminocarbonyl, N-butyl-N-(2-methylpropyl)aminocarbonyl, N-butyl-N-(1,1-dimethylethyl)aminocarbonyl, N-(1-methylpropyl)-N-(2-methylpropyl)aminocarbonyl, N-(1,1-dimethylethyl)-N-(1-methylpropyl)aminocarbonyl or N-(1,1-dimethylethyl)-N-(2-methylpropyl)aminocarbonyl;

di(C₁-C₄-alkyl)aminocarbonyl-C₁-C₄-alkyl: C₁-C₄-alkyl which is monosubstituted by di(C₁-C₄-alkyl)aminocarbonyl, for example di(C₁-C₄-alkyl)aminocarbonylmethyl, 1- or 2-di(C₁-C₄-alkyl)aminocarbonylethyl, 1-, 2- or 3-di(C₁-C₄-alkyl)aminocarbonylpropyl;

di(C₁-C₄-alkyl)aminocarbonyl-C₁-C₄-alkoxy: C₁-C₄-alkoxy which is monosubstituted by di(C₁-C₄-alkyl)aminocarbonyl, for example di(C₁-C₄-alkyl)aminocarbonylmethoxy, 1- or 2-di(C₁-C₄-alkyl)aminocarbonylethoxy, 1-, 2- or 3-di(C₁-C₄-alkyl)aminocarbonylpropoxy;

di(C₁-C₄-alkyl)aminocarbonyl-C₁-C₄-alkylthio: C₁-C₄-alkylthio which is monosubstituted by di(C₁-C₄-alkyl)aminocarbonyl, for example di(C₁-C₄-alkyl)aminocarbonylmethylthio, 1- or 2-di(C₁-C₄-alkyl)aminocarbonylethylthio, 1-, 2- or 3-di(C₁-C₄-alkyl)aminocarbonylpropylthio;

C₂-C₆-alkenyl: vinyl, prop-1-en-1-yl, allyl, 1-methylethenyl, 1-buten-1-yl, 1-buten-2-yl, 1-buten-3-yl, 2-buten-1-yl, 1-methylprop-1-en-1-yl, 2-methylprop-1-en-1-yl, 1-methylprop-2-en-1-yl, 2-methylprop-2-en-1-yl, n-penten-1-yl, n-penten-2-yl, n-penten-3-yl, n-penten-4-yl, 1-methylbut-1-en-1-yl, 2-methylbut-1-en-1-yl, 3-methylbut-1-en-1-yl, 1-methylbut-2-en-1-yl, 2-methylbut-2-en-1-yl, 3-methylbut-2-en-1-yl, 1-methylbut-3-en-1-yl, 2-methylbut-3-en-1-yl, 3-methylbut-3-en-1-yl, 1,1-dimethylprop-2-en-1-yl, 1,2-dimethylprop-1-en-1-yl, 1,2-dimethylprop-2-en-1-yl, 1-ethylprop-1-en-2-yl, 1-ethylprop-2-en-1-yl, n-hex-1-en-1-yl, n-hex-2-en-1-yl, n-hex-3-en-1-yl, n-hex-4-en-1-yl, n-hex-5-en-1-yl, 1-methylpent-1-en-1-yl, 2-methylpent-1-en-1-yl, 3-methylpent-1-en-1-yl, 4-methylpent-1-en-1-yl, 1-methylpent-2-en-1-yl, 2-methylpent-2-en-1-yl, 3-methylpent-2-en-1-yl, 4-methylpent-2-en-1-yl, 1-methylpent-3-en-1-yl, 2-methylpent-3-en-1-yl, 3-methylpent-3-en-1-yl, 4-methylpent-3-en-1-yl, 1-methylpent-4-en-1-yl, 2-methylpent-4-en-1-yl, 3-methylpent-4-en-1-yl, 4-methylpent-4-en-1-yl, 1,1-dimethylbut-2-en-1-yl, 1,1-dimethylbut-3-en-1-yl, 1,2-dimethylbut-1-en-1-yl, 1,2-dimethylbut-2-en-1-yl, 1,2-dimethylbut-3-en-1-yl, 1,3-dimethylbut-1-en-1-yl, 1,3-dimethylbut-2-en-1-yl, 1,3-dimethylbut-3-en-1-yl, 2,2-dimethylbut-3-en-1-yl, 2,3-dimethylbut-1-en-1-yl, 2,3-dimethylbut-2-en-1-yl, 2,3-dimethylbut-3-en-1-yl, 3,3-dimethylbut-1-en-1-yl, 3,3-dimethylbut-2-en-1-yl, 1-ethylbut-1-en-1-yl, 1-ethylbut-2-en-1-yl, 1-ethylbut-3-en-1-yl, 2-ethylbut-1-en-1-yl, 2-ethylbut-2-en-1-yl, 2-ethylbut-3-en-1-yl, 1,1,2-trimethylprop-2-en-1-yl, 1-ethyl-1-methylprop-2-en-1-yl, 1-ethyl-2-methylprop-1-en-1-yl or 1-ethyl-2-methylprop-2-en-1-yl;

C₂-C₆-haloalkenyl: C₂-C₆-alkenyl as mentioned above which is partially or fully substituted by fluorine, chlorine and/or bromine, i.e., for example, 2-chlorovinyl, 2-chloroallyl, 3-chloroallyl, 2,3-dichloroallyl, 3,3-dichloroallyl, 2,3,3-trichloroallyl, 2,3-dichlorobut-2-enyl, 2-bromoallyl, 3-bromoallyl, 2,3-dibromoallyl, 3,3-dibromoallyl, 2,3,3-tribromoallyl and 2,3-dibromobut-2-enyl, preferably C₃— or C₄-haloalkenyl;

C₂-C₆-alkynyl: ethynyl and C₃-C₆-alkynyl such as prop-1-yn-1-yl, prop-2-yn-1-yl, n-but-1-yn-1-yl, n-but-1-yn-3-yl, n-but-1-yn-4-yl, n-but-2-yn-1-yl, n-pent-1-yn-1-yl, n-pent-1-yn-3-yl, n-pent-1-yn-4-yl, n-pent-1-yn-5-yl, n-pent-2-yn-1-yl, n-pent-2-yn-4-yl, n-pent-2-yn-5-yl, 3-methylbut-1-yn-3-yl, 3-methylbut-1-yn-4-yl, n-hex-1-yn-1-yl, n-hex-1-yn-3-yl, n-hex-1-yn-4-yl, n-hex-1-yn-5-yl, n-hex-1-yn-6-yl, n-hex-2-yn-1-yl, n-hex-2-yn-4-yl, n-hex-2-yn-5-yl, n-hex-2-yn-6-yl, n-hex-3-yn-1-yl, n-hex-3-yn-2-yl, 3-methylpent-1-yn-1-yl, 3-methylpent-1-yn-3-yl, 3-methylpent-1-yn-4-yl, 3-methylpent-1-yn-5-yl, 4-methylpent-1-yn-1-yl, 4-methylpent-2-yn-4-yl or 4-methylpent-2-yn-5-yl, preferably prop-2-yn-1-yl;

C₂-C₆-haloalkynyl: C₂-C₆-alkynyl as mentioned above which is partially or fully substituted by fluorine, chlorine and/or bromine, i.e., for example, 1,1-difluoroprop-2-yn-1-yl, 1,1-difluorobut-2-yn-1-yl, 4-fluorobut-2-yn-1-yl, 4-chlorobut-2-yn-1-yl, 5-fluoropent-3-yn-1-yl or 6-fluorohex-4-yn-1-yl, preferably C₃— or C₄-haloalkynyl;

C₃-C₈-cycloalkyl: cyclopropyl, cyclobutyl, cyclopentyl, cyclohexyl, cycloheptyl or cyclooctyl;

C₃-C₈-cycloalkyl which contains a carbonyl or thiocarbonyl ring member, for example cyclobutanon-2-yl, cyclobutanon-3-yl, cyclopentanon-2-yl, cyclopentanon-3-yl, cyclohexanon-2-yl, cyclohexanon-4-yl, cycloheptanon-2-yl, cyclooctanon-2-yl, cyclobutanethion-2-yl, cyclobutanethion-3-yl, cyclopentanethion-2-yl, cyclopentanethion-3-yl, cyclohexanethion-2-yl, cyclohexanethion-4-yl, cycloheptanethion-2-yl or cyclooctanethion-2-yl, preferably cyclopentanon-2-yl or cyclohexanon-2-yl;

C₃-C₈-cycloalkyl-C₁-C₄-alkyl: cyclopropylmethyl, 1-cyclopropylethyl, 2-cyclopropylethyl, 1-cyclopropylprop-1-yl, 2-cyclopropylprop-1-yl, 3-cyclopropylprop-1-yl, 1-cyclopropylbut-1-yl, 2-cyclopropylbut-1-yl, 3-cyclopropylbut-1-yl, 4-cyclopropylbut-1-yl, 1-cyclopropylbut-2-yl, 2-cyclopropylbut-2-yl, 3-cyclopropylbut-2-yl, 4-cyclopropylbut-2-yl, 1-(cyclopropylmethyl)eth-1-yl, 1-(cyclopropylmethyl)-1-(methyl)eth-1-yl, 1-(cyclopropylmethyl)prop-1-yl, cyclobutylmethyl, 1-cyclobutylethyl, 2-cyclobutylethyl, 1-cyclobutylprop-1-yl, 2-cyclobutylprop-1-yl, 3-cyclobutylprop-1-yl, 1-cyclobutylbut-1-yl, 2-cyclobutylbut-1-yl, 3-cyclobutylbut-1-yl, 4-cyclobutylbut-1-yl, 1-cyclobutylbut-2-yl, 2-cyclobutylbut-2-yl, 3-cyclobutylbut-2-yl, 4-cyclobutylbut-2-yl, 1-(cyclobutylmethyl)eth-1-yl, 1-(cyclobutylmethyl)-1-(methyl)eth-1-yl, 1-(cyclobutylmethyl)prop-1-yl, cyclopentylmethyl, 1-cyclopentylethyl, 2-cyclopentylethyl, 1-cyclopentylprop-1-yl, 2-cyclopentylprop-1-yl, 3-cyclopentylprop-1-yl, 1-cyclopentylbut-1-yl, 2-cyclopentylbut-1-yl, 3-cyclopentylbut-1-yl, 4-cyclopentylbut-1-yl, 1-cyclopentylbut-2-yl, 2-cyclopentylbut-2-yl, 3-cyclopentylbut-2-yl, 4-cyclopentylbut-2-yl, 1-(cyclopentylmethyl)eth-1-yl, 1-(cyclopentylmethyl)-1-(methyl)eth-1-yl, 1-(cyclopentylmethyl)prop-1-yl, cyclohexylmethyl, 1-cyclohexylethyl, 2-cyclohexylethyl, 1-cyclohexylprop-1-yl, 2-cyclohexylprop-1-yl, 3-cyclohexylprop-1-yl, 1-cyclohexylbut-1-yl, 2-cyclohexylbut-1-yl, 3-cyclohexylbut-1-yl, 4-cyclohexylbut-1-yl, 1-cyclohexylbut-2-yl, 2-cyclohexylbut-2-yl, 3-cyclohexylbut-2-yl, 4-cyclohexylbut-2-yl, 1-(cyclohexylmethyl)eth-1-yl, 1-(cyclohexylmethyl)-1-(methyl)eth-1-yl, 1-(cyclohexylmethyl)prop-1-yl, cycloheptylmethyl, 1-cycloheptylethyl, 2-cycloheptylethyl, 1-cycloheptylprop-1-yl, 2-cycloheptylprop-1-yl, 3-cycloheptylprop-1-yl, 1-cycloheptylbut-1-yl, 2-cycloheptylbut-1-yl, 3-cycloheptylbut-1-yl, 4-cycloheptylbut-1-yl, 1-cycloheptylbut-2-yl, 2-cycloheptylbut-2-yl, 3-cycloheptylbut-2-yl, 4-cycloheptylbut-2-yl, 1-(cycloheptylmethyl)eth-1-yl, 1-(cycloheptylmethyl)-1-(methyl)eth-1-yl, 1-(cycloheptylmethyl)prop-1-yl, cyclooctylmethyl, 1-cyclooctylethyl, 2-cyclooctylethyl, 1-cyclooctylprop-1-yl, 2-cyclooctylprop-1-yl, 3-cyclooctylprop-1-yl, 1-cyclooctylbut-1-yl, 2-cyclooctylbut-1-yl, 3-cyclooctylbut-1-yl, 4-cyclooctylbut-1-yl, 1-cyclooctylbut-2-yl, 2-cyclooctylbut-2-yl, 3-cyclooctylbut-2-yl, 4-cyclooctylbut-2-yl, 1-(cyclooctylmethyl)eth-1-yl, 1-(cyclooctylmethyl)-1-(methyl)eth-1-yl or 1-(cyclooctylmethyl)prop-1-yl, preferably cyclopropylmethyl, cyclobutylmethyl, cyclopentylmethyl or cyclohexylmethyl;

C₃-C₈-cycloalkyl-C₁-C₄-alkyl which contains a carbonyl or thiocarbonyl ring member, for example cyclobutanon-2-ylmethyl, cyclobutanon-3-ylmethyl, cyclopentanon-2-ylmethyl, cyclopentanon-3-ylmethyl, cyclohexanon-2-ylmethyl, cyclohexanon-4-ylmethyl, cycloheptanon-2-ylmethyl, cyclooctanon-2-ylmethyl, cyclobutanethion-2-ylmethyl, cyclobutanethion-3-ylmethyl, cyclopentanethion-2-ylmethyl, cyclopentanethion-3-ylmethyl, cyclohexanethion-2-ylmethyl, cyclohexanethion-4-ylmethyl, cycloheptanethion-2-ylmethyl, cyclooctanethion-2-ylmethyl, 1-(cyclobutanon-2-yl)ethyl, 1-(cyclobutanon-3-yl)ethyl, 1-(cyclopentanon-2-yl)ethyl, 1-(cyclopentanon-3-yl)ethyl, 1-(cyclohexanon-2-yl)ethyl, 1-(cyclohexanon-4-yl)ethyl, 1-(cycloheptanon-2-yl)ethyl, 1-(cyclooctanon-2-yl)ethyl, 1-(cyclobutanethion-2-yl)ethyl, 1-(cyclobutanethion-3-yl)ethyl, 1-(cyclopentanethion-2-yl)ethyl, 1-(cyclopentanethion-3-yl)ethyl, 1-(cyclohexanethion-2-yl)ethyl, 1-(cyclohexanethion-4-yl ) ethyl, 1-(cycloheptanethion-2-yl)ethyl, 1-(cyclooctanethion-2-yl)ethyl, 2-(cyclobutanon-2-yl)ethyl, 2-(cyclobutanon-3-yl)ethyl, 2-(cyclopentanon-2-yl)ethyl, 2-(cyclopentanon-3-yl)ethyl, 2-(cyclohexanon-2-yl)ethyl, 2-(cyclohexanon-4-yl)ethyl, 2-(cycloheptanon-2-yl)ethyl, 2-(cyclooctanon-2-yl)ethyl, 2-(cyclobutanethion-2-yl)ethyl, 2-(cyclobutanethion-3-yl)ethyl, 2-(cyclopentanethion-2-yl)ethyl, 2-(cyclopentanethion-3-yl)ethyl, 2-(cyclohexanethion-2-yl)ethyl, 2-(cyclohexanethion-4-yl)ethyl, 2-(cycloheptanethion-2-yl)ethyl, 2-(cyclooctanethion-2-yl)ethyl, 3-(cyclobutanon-2-yl)propyl, 3-(cyclobutanon-3-yl)propyl, 3-(cyclopentanon-2-yl)propyl, 3-(cyclopentanon-3-yl)propyl, 3-(cyclohexanon-2-yl)propyl, 3-(cyclohexanon-4-yl)propyl, 3-(cycloheptanon-2-yl)propyl, 3-(cyclooctanon-2-yl)propyl, 3-(cyclobutanethion-2-yl)propyl, 3-(cyclobutanethion-3-yl)propyl, 3-(cyclopentanethion-2-yl)propyl, 3-(cyclopentanethion-3-yl)propyl, 3-(cyclohexanethion-2-yl)propyl, 3-(cyclohexanethion-4-yl)propyl, 3-(cycloheptanethion-2-yl)propyl, 3-(cyclooctanethion-2-yl)propyl, 4-(cyclobutanon-2-yl)butyl, 4-(cyclobutanon-3-yl)butyl, 4-(cyclopentanon-2-yl)butyl, 4-(cyclopentanon-3-yl)butyl, 4-(cyclohexanon-2-yl)butyl, 4-(cyclohexanon-4-yl)butyl, 4-(cycloheptanon-2-yl)butyl, 4-(cyclooctanon-2-yl)butyl, 4-(cyclobutanethion-2-yl)butyl, 4-(cyclobutanethion-3-yl)butyl, 4-(cyclopentanethion-2-yl)butyl, 4-(cyclopentanethion-3-yl)butyl, 4-(cyclohexanethion-2-yl)butyl, 4-(cyclohexanethion-4-yl)butyl, 4-(cycloheptanethion-2-yl)butyl or 4-(cyclooctanethion-2-yl)butyl, preferably cyclopentanon-2-ylmethyl, cyclohexanon-2-ylmethyl, 2-(cyclopentanon-2-yl)ethyl or 2-(cyclohexanon-2-yl)ethyl.

3- to 7-membered heterocyclyl is understood as meaning not only saturated, partially or fully unsaturated, but also aromatic, heterocycles with one, two or three hetero atoms, the hetero atoms being selected from among nitrogen atoms, oxygen and sulfur atoms. Saturated 3- to 7-membered heterocyclyl may also contain a carbonyl or thiocarbonyl ring member.

Examples of saturated heterocycles which may contain a carbonyl or thiocarbonyl ring member are:

oxiranyl, thiiranyl, aziridin-1-yl, aziridin-2-yl, diaziridin-1-yl, diaziridin-3-yl, oxetan-2-yl, oxetan-3-yl, thietan-2-yl, thietan-3-yl, azetidin-1-yl, azetidin-2-yl, azetidin-3-yl, tetrahydrofuran-2-yl, tetrahydrofuran-3-yl, tetrahydrothiophen-2-yl, tetrahydrothiophen-3-yl, pyrrolidin-1-yl, pyrrolidin-2-yl, pyrrolidin-3-yl, 1,3-dioxolan-2-yl, 1,3-dioxolan-4-yl, 1,3-oxathiolan-2-yl, 1,3-oxathiolan-4-yl, 1,3-oxathiolan-5-yl, 1,3-oxazolidin-2-yl, 1,3-oxazolidin-3-yl, 1,3-oxazolidin-4-yl, 1,3-oxazolidin-5-yl, 1,2-oxazolidin-2-yl, 1,2-oxazolidin-3-yl, 1,2-oxazolidin-4-yl, 1,2-oxazolidin-5-yl, 1,3-dithiolan-2-yl, 1,3-dithiolan-4-yl, tetrahydropyrazol-1-yl, tetrahydropyrazol-3-yl, tetrahydropyrazol-4-yl, tetrahydropyran-2-yl, tetrahydropyran-3-yl, tetrahydropyran-4-yl, tetrahydrothiopyran-2-yl, tetrahydrothiopyran-3-yl, tetrahydropyran-4-yl, piperidin-1-yl, piperidin-2-yl, piperidin-3-yl, piperidin-4-yl, 1,3-dioxan-2-yl, 1,3-dioxan-4-yl, 1,3-dioxan-5-yl, 1,4-dioxan-2-yl, 1,3-oxathian-2-yl, 1,3-oxathian-4-yl, 1,3-oxathian-5-yl, 1,3-oxathian-6-yl, 1,4-oxathian-2-yl, 1,4-oxathian-3-yl, morpholin-2-yl, morpholin-3-yl, morpholin-4-yl, hexahydropyridazin-1-yl, hexahydropyridazin-3-yl, hexahydropyridazin-4-yl, hexahydropyrimidin-1-yl, hexahydropyrimidin-2-yl, hexahydropyrimidin-4-yl, hexahydropyrimidin-5-yl, piperazin-1-yl, piperazin-2-yl, piperazin-3-yl, hexahydro-1,3,5-triazin-1-yl, hexahydro-1,3,5-triazin-2-yl, oxepan-2-yl, oxepan-3-yl, oxepan-4-yl, thiepan-2-yl, thiepan-3-yl, thiepan-4-yl, 1,3-dioxepan-2-yl, 1,3-dioxepan-4-yl, 1,3-dioxepan-5-yl, 1,3-dioxepan-6-yl, 1,3-dithiepan-2-yl, 1,3-dithiepan-4-yl, 1,3-dithiepan-5-yl, 1,3-dithiepan-6-yl, 1,4-dioxepan-2-yl, 1,4-dioxepan-7-yl, hexahydroazepin-1-yl, hexahydroazepin-2-yl, hexahydroazepin-3-yl, hexahydroazepin-4-yl, hexahydro-1,3-diazepin-1-yl, hexahydro-1,3-diazepin-2-yl, hexahydro-1,3-diazepin-4-yl, hexahydro-1,4-diazepin-1-yl and hexahydro-1,4-diazepin-2-yl.

Examples of unsaturated heterocycles which may contain a carbonyl or thiocarbonyl ring member are:

dihydrofuran-2-yl, 1,2-oxazolin-3-yl, 1,2-oxazolin-5-yl, 1,3-oxazolin-2-yl.

Examples of aromatic heterocyclyl are the 5- and 6-membered aromatic, heterocyclic radicals, for example furyl such as 2-furyl and 3-furyl, thienyl such as 2-thienyl and 3-thienyl, pyrrolyl such as 2-pyrrolyl and 3-pyrrolyl, isoxazolyl such as 3-isoxazolyl, 4-isoxazolyl and 5-isoxazolyl, isothiazolyl such as 3-isothiazolyl, 4-isothiazolyl and 5-isothiazolyl, pyrazolyl such as 3-pyrazolyl, 4-pyrazolyl and 5-pyrazolyl, oxazolyl such as 2-oxazolyl, 4-oxazolyl and 5-oxazolyl, thiazolyl such as 2-thiazolyl, 4-thiazolyl and 5-thiazolyl, imidazolyl such as 2-imidazolyl and 4-imidazolyl, oxadiazolyl such as 1,2,4-oxadiazol-3-yl, 1,2,4-oxadiazol-5-yl and 1,3,4-oxadiazol-2-yl, thiadiazolyl such as 1,2,4-thiadiazol-3-yl, 1,2,4-thiadiazol-5-yl and 1,3,4-thiadiazol-2-yl, triazolyl such as 1,2,4-triazol-1-yl, 1,2,4-triazol-3-yl and 1,2,4-triazol-4-yl, pyridinyl such as 2-pyridinyl, 3-pyridinyl and 4-pyridinyl, pyridazinyl such as 3-pyridazinyl and 4-pyridazinyl, pyrimidinyl such as 2-pyrimidinyl, 4-pyrimidinyl and 5-pyrimidinyl, furthermore 2-pyrazinyl, 1,3,5-triazin-2-yl and 1,2,4-triazin-3-yl, in particular pyridyl, pyrimidyl, furanyl and thienyl.

Examples of fused rings are, in addition to phenyl, the abovementioned heteroaromatic groups, in particular pyridine, pyrazine, pyridazine, pyrimidine, furan, dihydrofuran, thiophene, dihydrothiophene, pyrrole, dihydropyrrole, 1,3-dioxolane, 1,3-dioxolan-2-one, isoxazole, oxazole, oxazolinone, isothiazole, thiazole, pyrazole, pyrazoline, imidazole, imidazolinone, dihydroimidazole, 1,2,3-triazole, 1,1-dioxodihydroisothiazole, dihydro-1,4-dioxine, pyridone, dihydro-1,4-oxazine, dihydro-1,4-oxazin-2-one, dihydro-1,4-oxazin-3-one, dihydro-1,3-oxazine, dihydro-1,3-thiazin-2-one, dihydro-1,4-thiazine, dihydro-1,4-thiazin-2-one, dihydro-1,4-thiazin-3-one, dihydro-1,3-thiazine and dihydro-1,3-thiazin-2-one, which, in turn, can have one, two or three substituents. Examples of suitable substituents on the fused ring are the meanings given hereinbelow for R¹⁵, R¹⁶, R¹⁷ and R¹⁸.

With regard to the use of the 2-aryl-5-trifluoromethylpyridines I as herbicides or desiccants/defoliants, those compounds I are preferred in which R² is fluorine or chlorine. R¹ is preferably methyl. Furthermore preferred compounds I are those in which the variables R³ and R⁴ have the following meanings, in each case alone or in combination:

R³ is hydrogen, chlorine or, in particular, fluorine,

R⁴ is halogen, preferably chlorine, and cyano.

In the compounds in which R⁶ is hydrogen and X—R⁵ together with R⁴ do not form a chain (hereinbelow compounds IA), X, R⁴ and R⁵ independently of one another and preferably together have the following preferred meanings:

R⁴ is chlorine or cyano,

X is a chemical bond, methylene, ethane-1,2-diyl, ethene-1,2-diyl which can be unsubstituted or have attached to it a substituent selected from among C₁-C₄-alkyl, specifically methyl, or halogen, specifically chlorine, for example 1- or 2-chloroethane-1,2-diyl, 1- or 2-chloroethene-1,2-diyl, 1- or 2-bromoethane-1,2-diyl, 1- or 2-bromoethene-1,2-diyl, 1- or 2-methylethane-1,2-diyl, 1- or 2-methylethene-1,2-diyl, in particular a chemical bond, 1- or 2-chloroethane-1,2-diyl, 1- or 2-chloroethene-1,2-diyl, 1- or 2-bromoethene-1,2-diyl, 1- or 2-methylethene-1,2-diyl. If X is substituted ethane-1,2-diyl, ethene-1,2-diyl, the substituent is preferably attached to the carbon atom which is adjacent to the group R⁵;

R⁵ is hydrogen, fluorine, nitro, chlorosulfonyl, —O—Y—R⁷, —O—CO—Y—R⁷, —N(Y—R⁷) (Z-R⁸), —N(Y—R⁷)—SO₂-Z-R⁸, —N(SO₂—Y—R⁷)(SO₂-Z-R⁸), —S—Y—R⁷, —SO₂—N(Y—R⁷)(Z-R⁸), —C(═NOR⁹)—Y—R⁷, —C(═NOR⁹) Y—R⁷, —C Y—R⁷, PO(O—Y—R⁷) or —CO—N(Y—R⁷)(Z-R⁸), in particular —O—Y—R⁷, —S—Y—R⁷, —N(Y—R⁷)—SO₂-Z-R⁸ or —C Y—R⁷,

especially preferably —CO——O—Y—R⁷ and —O—Y—R⁷.

The variables R⁷, R⁸, R⁹, Y, Z mentioned in the definition of the variables R⁵ preferably have the following meanings:

Y, Z independently of one another are a chemical bond or methylene;

R⁷, R⁸ independently of one another are

hydrogen, C₁-C₄-haloalkyl, C₂-C₆-alkenyl, C₂-C₆-alkynyl, —CH(R¹⁰) (R¹¹), C₁-C₄-alkoxy-C₁-C₄-alkyl, —C(R¹⁰) (R¹¹)—N(R¹²)R¹³, —C(R¹⁰)(R¹¹)—CO—OR¹², —C(R¹⁰)(R¹¹)—CO—N(R¹²)R¹³, C₃-C₈-cycloalkyl or phenyl, it being possible for the cycloalkyl ring and the phenyl ring to be unsubstituted or to have attached to it one or two substituents, in each case selected from the group consisting of cyano, nitro, halogen, C₁-C₄-alkyl, C₁-C₄-alkoxy, C₁-C₄-alkylsulfonyl, (C₁-C₄-alkyl)carbonyl, (C₁-C₄-alkyl)carbonyloxy and (C₁-C₄-alkoxy)carbonyl;

in particular hydrogen, C₁-C₆-haloalkyl, C₁-C₄-alkoxy-C₁-C₄-alkyl, C₂-C₆-alkenyl, C₂-C₆-alkynyl, —CH(R¹⁰) (R¹¹), —C(R¹⁰)(R¹¹)—CO—OR¹², —C(R¹⁰)(R¹¹)—CO—N(R¹²)R¹³, phenyl or C₃-C₈-Cycloalkyl, especially preferably hydrogen, C₁-C₆-alkyl, C₁-C₄-alkoxy-C₁-C₄-alkyl, C₂-C₆-alkenyl, C₂-C₆-alkynyl, —C(R¹⁰) (R¹¹)—CO—OR¹², or C₃-C₈-Cycloalkyl.

In this context, the variables R¹⁰, R¹¹, R¹² and R¹³ independently of one another preferably have the meanings stated hereinbelow:

R¹⁰ is hydrogen or C₁-C₄-alkyl, specifically methyl or ethyl;

R¹¹ is hydrogen or C₁-C₄-alkyl, specifically methyl or ethyl;

R¹², R¹³ independently of one another are hydrogen, C₁-C₆-alkyl, C₁-C₆-haloalkyl, C₂-C₆-alkenyl, C₂-C₆-alkynyl, C₃-C₈-cycloalkyl, C₃-C₈-cycloalkyl-C₁-C₄-alkyl, or C₁-C₄-alkoxy-C₁-C₄-alkyl, in particular hydrogen or C₁-C₆-alkyl;

R⁹ is C₁-C₆-alkyl, C₁-C₄-alkoxycarbonyl-C₁-C₄-alkyl, C₂-C₆-alkenyl, in particular methyl or ethyl.

In a further preferred embodiment R⁷ and R⁸ independently of one another are C₃-C₈-cycloalkyl-C₁-C₄-alkyl or C₁-C₆-alkyl.

R⁵ is very especially preferably C₃-C₄-alkynyloxy, C₁-C₄-alkoxy, C₃-C₄-alkenyloxy, OCH(R¹⁹)—COOR²⁰, CO—OR²¹ or COO—CH(R²²)COOR²³, where

R¹⁹, R²² independently of one another are hydrogen or C₁-C₄-alkyl,

R²⁰, R²¹, R²³ independently of one another are C₁-C₄-alkyl, C₃-C₆-alkenyl, C₃-C₆-alkynyl, C₁-C₄-haloalkyl, C₁-C₄-alkoxy-C₁-C₄-alkyl;

in particular when X is a single bond.

Those compounds IA in which X is a single bond and R⁵ is COO—CH(R²²)COOR²³, where R²² and R²³ independently of one another are C₁-C₄-alkyl, have a very particularly high activity, in particular when the carbon atom to which the group R²² is attached is in S configuration.

R⁴ and XR⁵ or XR⁵ and R⁶ in formula I may also form a 3- or 4-membered chain which, in addition to carbon, can have 1, 2 or 3, preferably 2, hetero atoms selected from among nitrogen, oxygen and sulfur atoms, which chain can be unsubstituted or, in turn, have attached to it one, two or three substituents and whose members can also encompass one or two nonadjacent carbonyl, thiocarbonyl or sulfonyl groups. Such compounds are termed compounds IB and IC hereinbelow.

Examples are compounds IB where R⁴ together with X—R⁵ in formula I are a chain of the formulae: —O—C(R¹⁵, R¹⁶) N—CO—N(R¹⁷)—, —S—C(R¹⁵, R¹⁶)ₙ—CO—N(R¹⁷)—, —O—C(R¹⁵, R¹⁶)ₙ—CS—N(R¹⁷)—, —S—C(R¹⁵, R¹⁶)ₙ—CS—N(R¹⁷)—, —N═C(R¹⁸)—O— or —N═C(R¹⁸)—S— (compounds IB) in which the variables n, R¹⁵ to R¹⁸ have the following meanings:

n is 0 or 1, in particular 1,

R¹⁵, R¹⁶ independently of one another are

hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;

R¹⁷ is hydrogen, hydroxyl, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylsulfonyl, C₁-C₄-haloalkylsulfonyl, C₁-C₄-alkylcarbonyl, C₁-C₄-haloalkylcarbonyl, C₁-C₄-alkoxycarbonyl, C₁-C₄-alkoxy-C₁-C₄-alkyl, C₁-C₄-alkoxycarbonyl-C₁-C₄-alkyl, C₁-C₄-alkoxycarbonyl-C₁-C₄-alkoxy, mono- and di(C₁-C₄-alkyl)aminocarbonyl, mono- and di(C₁-C₄-alkyl)aminocarbonyl-C₁-C₄-alkyl, mono- and di(C₁-C₄-alkyl)aminocarbonyl-C₁-C₄-alkoxy, phenyl, phenyl-C₁-C₄-alkyl, C₃-C₈-cycloalkyl, C₃-C₈-cycloalkyl-C₁-C₄-alkyl, 3-, 4-, 5-, 6- or 7-membered heterocyclyl, 3-, 4-, 5-, 6- or 7-membered heterocyclyl-C₁-C₄-alkyl, preferably 5- or 6-membered, preferably saturated, heterocyclyl which has in each case one or two, preferably one, ring hetero atom selected from among oxygen, nitrogen or sulfur;

R¹⁸ is hydrogen, halogen, cyano, amino, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylamino, di(C₁-C₄-alkyl)amino, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkylcarbonyl, C₁-C₄-haloalkylcarbonyl, C₁-C₄-alkoxy-C₁-C₄-alkyl, C₁-C₄-alkoxycarbonyl, C₁-C₄-alkoxycarbonyl-C₁-C₄-alkyl, C₁-C₄-alkoxycarbonyl-C₁-C₄-alkoxy, C₁-C₄-alkoxycarbonyl-C₁-C₄-alkylthio, di(C₁-C₄-alkyl)aminocarbonyl, di(C₁-C₄-alkyl)aminocarbonyl-C₁-C₄-alkyl, di(C₁-C₄-alkyl)aminocarbonyl-C₁-C₄-alkoxy, di (C₁-C₄-alkyl) aminocarbonyl-C₁-C₄-alkylthio, C₃-C₈-cycloalkyl, phenyl, phenyl-C₁-C₄-alkyl, C₃-C₈-cycloalkyl-C₁-C₄-alkyl, 3-, 4-, 5-, 6- or 7-membered, preferably 5- or 6-membered, preferably saturated, heterocyclyl which has one or two, preferably one, ring hetero atom selected from among oxygen, nitrogen or sulfur.

The variables R¹⁵ to R¹⁸ preferably have the following meanings:

R¹⁵, R¹⁶ independently of one another are hydrogen or methyl;

R¹⁷ is hydrogen, hydroxyl, C₁-C₄-alkyl, C₁-C₄-haloalkyl, C₂-C₆-alkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkoxycarbonyl-C₁-C₄-alkyl, C₁-C₄-alkoxycarbonyl-C₁-C₄-alkoxy, C₃-C₈-cycloalkyl, C₃-C₈-cycloalkyl-C₁-C₄-alkyl or phenyl-C₁-C₄-alkyl or 3-, 4-, 5- or 6-membered, preferably 5- or 6-membered, preferably saturated, heterocyclyl which has one ring hetero atom selected from among oxygen, nitrogen or sulfur;

R¹⁸ is hydrogen, halogen, amino, C₁-C₆-alkyl, C₁-C₆-haloalkyl, C₂-C₆-alkenyl, C₂-C₆-haloalkenyl, C₂-C₆-alkynyl, C₁-C₄-alkoxy, C₃-C₆-alkenyloxy, C₃-C₆-alkynyloxy, C₁-C₄-alkylamino, di(C₁-C₄-alkyl)amino, C₁-C₄-alkylthio, C₁-C₄-alkoxycarbonyl-C₁-C₄-alkyl, C₁-C₄-alkoxycarbonyl-C₁-C₄-alkoxy, C₁-C₄-alkoxycarbonyl-C₁-C₄-alkylthio, C₃-C₈-cycloalkyl, phenyl, phenyl-C₁-C₄-alkyl, C₃-C₈-cycloalkyl-C₁-C₄-alkyl, 3-, 4-, 5- or 6-membered, preferably 5- or 6-membered, preferably saturated, heterocyclyl which has one ring hetero atom selected from among oxygen, nitrogen or sulfur.

Especially preferred among the compounds IB are those compounds in which R⁴ together with X—R⁵ is a chain of the formula —O—[C(R¹⁵)(R¹⁶)]ₙ—CO—N(R¹⁷)—, —S—[C(R¹⁵)(R¹⁶)]ₙ—CO—N(R¹⁷)— where n=0 or 1. R¹⁵ and R¹⁷ in particular have the meanings mentioned as being preferred. Among them, very especially preferred compounds IB are those in which the nitrogen atom of the chain —O—C(R¹⁶)(R¹⁵)—CO—N(R¹⁷)— or —S—C(R¹⁶)(R¹⁵)—CO—N(R¹⁷)—is bonded to the carbon atom of the phenyl ring in formula I which has the group X—R⁵ attached to it (meta position relative to the pyridine group). R¹⁶ in these chains is preferably hydrogen. In the compounds IB, R³ is preferably halogen and in particular fluorine, or else hydrogen.

Examples of compounds IC are those compounds of the formula I in which R⁶ together with X—R⁵ is a chain of the formulae: —O—(C(R¹⁵, R¹⁶))ₙ—CO—N(R¹⁷)—, —S—(C(R¹⁵, R¹⁶))ₙ-Co-N(R¹⁷)— where n=0 or 1, —N═C(R¹⁸)—O— or —N═C(R¹⁸)—S— (compounds IC).

In this context, the variables R¹⁵ to R¹⁸ have the meanings mentioned above, in particular the meanings mentioned as preferred. Preferred amongst these compounds are in particular those in which R⁶ together with X—R⁵ is a chain of the formula —N═C(R¹⁸)—O— or of the formula —N═C(R¹⁸)—S—.

In these compounds, the nitrogen atom of the chain is preferably bonded to the C atom of the phenyl ring in formula I which has the X—R⁵ group attached to it. In the compounds IC, R³ is preferably fluorine or hydrogen. R⁴ is preferably chlorine or cyano.

The 2-aryl-5-trifluoromethylpyridines according to the invention can be employed both as pyridines, where m assumes the value 0, or as pyridine-N-oxides, i.e. compounds of the formula I where m=1.

Especially preferred are the compounds of the formula IAa (compounds IA where m=0, R¹═CH₃ and R²═Cl, R³═F and R⁴═Cl) in which the variable X—R⁵ has the abovementioned meanings, in particular the meanings mentioned in each case one line of Table 1 (compounds IAa.1-IAa.232).

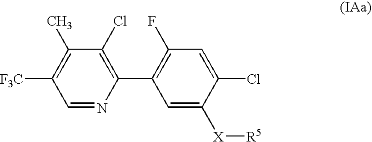

TABLE 1

| No. | X-R⁵ |
|---|---|
| 1 | H |
| 2 | OH |
| 3 | OCH₃ |
| 4 | OCH₂CH₃ |
| 5 | OCH₂CH₂Cl |
| 6 | OCH₂CH₂OCH₃ |
| 7 | OCH₂CH₂SCH₃ |
| 8 | OCH(CH₃)₂ |
| 9 | OCH₂CH═CH₂ |
| 10 | OCH₂CCH |
| 11 | OCH(CH₃)CCH |
| 12 | O—cyclopentyl |
| 13 | OCH₂COOH |
| 14 | OCH₂COOCH₃ |
| 15 | OCH₂COOCH₂CH₃ |
| 16 | OCH₂COOCH₂CH₂Cl |
| 17 | OCH₂COOCH₂CH₂OCH₃ |
| 18 | OCH₂COOCH₂CH₂SCH₃ |
| 19 | OCH₂COOCH(CH₃)₂ |
| 20 | OCH₂COOCH₂CH═CH₂ |
| 21 | OCH₂COOCH₂CCH |
| 22 | OCH₂COOCH₂COOCH₃ |
| 23 | OCH₂CONH₂ |
| 24 | OCH₂CONHCH₃ |
| 25 | OCH₂CON(CH₃)₂ |
| 26 | OCH₂CONH(OCH₃) |
| 27 | OCH₂CON(CH₃)(OCH₃) |
| 28 | OCH₂CONHCH₂COOCH₃ |
| 29 | OCH₂CON(CH₃)CH₂COOCH₃ |
| 30 | OCH₂CONHCH(CH₃)COOCH₃ |
| 31 | OCH₂CON(CH₃)CH(CH₃)COOCH₃ |
| 32 | OCH(CH₃)COOH |
| 33 | OCH(CH₃)COOCH₃ |
| 34 | OCH(CH₃)COOCH₂CH₃ |
| 35 | OCH(CH₃)COOCH₂CH₂Cl |
| 36 | OCH(CH₃)COOCH₂CH₂OCH₃ |
| 37 | OCH(CH₃)COOCH₂CH₂SCH₃ |
| 38 | OCH(CH₃)COOCH(CH₃)₂ |
| 39 | OCH(CH₃)COOCH₂CH═CH₂ |
| 40 | OCH(CH₃)COOCH₂CCH |
| 41 | OCH(CH₃)COOCH₂COOCH₃ |
| 42 | OCH(CH₃)CONH₂ |
| 43 | OCH(CH₃)CONHCH₃ |
| 44 | OCH(CH₃)CON(CH₃)₂ |
| 45 | OCH(CH₃)CONH(OCH₃) |
| 46 | OCH(CH₃)CON(CH₃)(OCH₃) |
| 47 | OCH(CH₃)CONHCH₂COOCH₃ |
| 48 | OCH(CH₃)CON(CH₃)CH₂COOCH₃ |
| 49 | OCH(CH₃)CONHCH(CH₃)COOCH₃ |
| 50 | OC(CH₃)₂COOCH₂CH═CH₂ |
| 51 | OC(CH₃)₂COOCH₂CCH |
| 52 | OC(CH₃)₂COOCH₂COOCH₃ |
| 53 | SH |
| 54 | SCH₃ |
| 55 | SCH₂CH₃ |
| 56 | SCH₂CH₂Cl |
| 57 | SCH₂CH₂OCH₃ |
| 58 | SCH(CH₃)₂ |
| 59 | SCH₂CH═CH₂ |
| 60 | SCH₂CCH |
| 61 | SCH(CH₃)CCH |
| 62 | S—cyclopentyl |
| 63 | SCH₂COOH |
| 64 | SCH₂COOCH₃ |
| 65 | SCH₂COOCH₂CH₃ |
| 66 | SCH₂COOCH₂CH₂Cl |
| 67 | SCH₂COOCH₂CH₂OCH₃ |
| 68 | SCH₂COOCH(CH₃)₂ |
| 69 | SCH₂COOCH₂CH═CH₂ |
| 70 | SCH₂COOCH₂CCH |
| 71 | SCH₂COOCH₂COOCH₃ |
| 72 | SCH(CH₃)COOCH₃ |
| 73 | SCH(CH₃)COOCH₂CH₃ |
| 74 | SCH(CH₃)COOCH₂CH═CH₂ |
| 75 | SCH(CH₃)COOCH₂CCH₃ |
| 76 | SCH(CH₃)COOCH₂COOCH₃ |
| 77 | SCH₂CONH₂ |
| 78 | SCH₂CONHCH₃ |
| 79 | SCH₂CON(CH₃)₂ |
| 80 | SCH₂CONHCH₂COOCH₃ |
| 81 | NO₂ |
| 82 | NHOH |
| 83 | NH₂ |
| 84 | N₃ |
| 85 | NHCH₃ |
| 86 | N(CH₃)₂ |
| 87 | NCH(CH₃)₂ |
| 88 | NHCH₂CH═CH₂ |
| 89 | N(CH₃)CH₂CCH |
| 90 | N(CH₃)CH₂CH═CH₂ |
| 91 | N(CH₃)CH₂CCH |
| 92 | N(CH₃)CH(CH₃)CCH |
| 93 | NHCH₂COOCH₃ |
| 94 | NHCH₂COOCH₂CH₃ |
| 95 | NHCH₂COOCH₂CH₂Cl |
| 96 | NHCH₂COOCH₂CH₂OCH₃ |
| 97 | NHCH₂COOCH₂CH═CH₂ |
| 98 | NHCH₂COOCH₂CCH |
| 99 | N(CH₃)CH₂COOCH₃ |
| 100 | N(CH₃)CH₂COOCH₂CH₃ |
| 101 | N(CH₃)CH₂COOCH₂CH₂Cl |
| 102 | N(CH₃)CH₂COOCH₂CH₂OCH₃ |
| 103 | N(CH₃)CH₂COOCH₂CH═CH₂ |
| 104 | N(CH₃)CH₂COOCH₂CCH |
| 105 | NHCH(CH₃)COOCH₃ |
| 106 | NHCH(CH₃)COOCH₂CH₃ |
| 107 | NHCH(CH₃)COOCH₂CH₂Cl |
| 108 | NHCH(CH₃)COOCH₂CH₂OCH₃ |
| 109 | NHCH(CH₃)COOCH₂CH═CH₂ |
| 110 | NHCH(CH₃)COOCH₂CCH |
| 111 | N(CH₃)CH(CH₃)COOCH₃ |
| 112 | N(CH₃)CH(CH₃)COOCH₂CH₃ |
| 113 | N(CH₃)CH(CH₃)COOCH₂CH₂Cl |
| 114 | N(CH₃)CH(CH₃)COOCH₂CH₂OCH₃ |
| 115 | N(CH₃)CH(CH₃)COOCH₂CH═CH₂ |
| 116 | N(CH₃)CH(CH₃)COOCH₂CCH |
| 117 | NHSO₂CH₃ |
| 118 | NHSO₂CH₂Cl |
| 119 | N(SO₂CH₃)₂ |
| 120 | NHSO₂CH₂CH₃ |
| 121 | N(SO₂CH₂CH₃)₂ |
| 122 | N(CH₃)SO₂CH₃ |
| 123 | N(CH₃)SO₂CH₂CH₃ |
| 124 | COOH |
| 125 | COCl |
| 126 | COOCH₃ |
| 127 | COOCH₂CH₃ |
| 128 | COOCH₂CH₂Cl |
| 129 | COOCH₂CH₂OCH₃ |
| 130 | COOCH₂CH₂SCH₃ |
| 131 | COOCH(CH₃)₂ |
| 132 | COOCH₂CH═CH₂ |
| 133 | COOCH₂CCH |
| 134 | COOCH₂COOH |
| 135 | COOCH₂COOCH₃ |
| 136 | COOCH₂COOCH₂CH₃ |
| 137 | COOCH₂COOCH₂CH₂Cl |
| 138 | COOCH₂COOCH₂CH₂OCH₃ |
| 139 | COOCH₂COOCH₂CH₂SCH₃ |
| 140 | COOCH₂COOCH₂CH═CH₂ |
| 141 | COOCH₂COOCH₂CCH |
| 142 | COOCH(CH₃)COOH |
| 143 | COOCH(CH₃)COOCH₃ |
| 144 | COOCH(CH₃)COOCH₂CH₃ |
| 145 | COOCH(CH₃)COOCH₂CH₂Cl |
| 146 | COOCH(CH₃)COOCH₂CH₂OCH₃ |
| 147 | COOCH(CH₃)COOCH₂CH₂SCH₃ |
| 148 | COOCH(CH₃)COOCH₂CH═CH₂ |
| 149 | COOCH(CH₃)COOCH₂CCH |
| 150 | COOC(CH₃)₂COOH |
| 151 | COOC(CH₃)₂COOCH₃ |
| 152 | COOC(CH₃)₂COOCH₂CH₃ |
| 153 | COOC(CH₃)₂COOCH₂CH═CH₂ |
| 154 | COOC(CH₃)₂COOCH₂CCH |
| 155 | CONH₂ |
| 156 | CONHCH₃ |
| 157 | CON(CH₃)₂ |
| 158 | CONH(OCH₃) |
| 159 | CON(CH₃)(OCH₃) |
| 160 | CONHCH₂COOCH₃ |
| 161 | CONHCH₂COOCH₂CH₃ |
| 162 | CONHCH₂COOCH₂CH₂Cl |
| 163 | CONHCH₂COOCH₂CH₂OCH₃ |
| 164 | CONHCH₂COOCH₂CH═CH₂ |
| 165 | CONHCH₂COOCH₂CCH |
| 166 | CON(CH₃)CH₂COOCH₃ |
| 167 | CON(CH₃)CH₂COOCH₂CH₃ |
| 168 | CON(CH₃)CH₂COOCH₂CH₂Cl |
| 169 | CON(CH₃)CH₂COOCH₂CH₂OCH₃ |
| 170 | CON(CH₃)CH₂COOCH₂CH═CH₂ |
| 171 | CON(CH₃)CH₂COOCH₂CCH |
| 172 | CONHCH(CH₃)COOCH₃ |
| 173 | CONHCH(CH₃)COOCH₂CH₃ |
| 174 | CONHCH(CH₃)COOCH₂CH₂Cl |
| 175 | CONHCH(CH₃)COOCH₂CH₂OCH₃ |
| 176 | CONHCH(CH₃)COOCH₂CH═CH₂ |
| 177 | CONHCH(CH₃)COOCH₂CCH |
| 178 | CON(CH₃)CH(CH₃)COOCH₃ |
| 179 | CON(CH₃)CH(CH₃)COOCH₂CH₃ |
| 180 | CON(CH₃)CH(CH₃)COOCH₂CH₂Cl |
| 181 | CON(CH₃)CH(CH₃)COOCH₂CH₂OCH₃ |
| 182 | CON(CH₃)CH(CH₃)COOCH₂CH═CH₂ |
| 183 | CON(CH₃)CH(CH₃)COOCH₂CCH |
| 184 | CONHSO₂CH₃ |
| 185 | CONHSO₂CH₂CH₃ |
| 186 | CH₂OH |
| 187 | CHO |
| 188 | CH═NOH |
| 189 | CH═NOCH₃ |
| 190 | CH═NOCH₂CH₃ |
| 191 | CH═NOCH₂COOCH₃ |
| 192 | CH═NOCH₂COOCH₂CH₃ |
| 193 | CH═NOCH(CH₃)COOCH₃ |
| 194 | C(═NOCH₃)(OCH₃) |
| 195 | C(═NOCH₃)(OCH₂COOCH₃) |
| 196 | C(═NOH)CH₃ |
| 197 | C(═NOCH₃)CH₃ |
| 198 | CH₂CHClCOOH |
| 199 | CH₂CHClCOOCH₃ |
| 200 | CH₂CHClCOOCH₂CH₃ |
| 201 | CH₂CHClCOOCH₂CH₂Cl |
| 202 | CH₂CHClCOOCH₂CH₂OCH₃ |
| 203 | CH₂CHClCOOCH₂COOCH₃ |
| 204 | CH₂CHClCONH₂ |
| 205 | CH₂CHClCONHCH₃ |
| 206 | CH₂CHClCON(CH₃)₂ |
| 207 | CH₂CHClCONCH₂COOCH₃ |
| 208 | CH═CHCOOH |
| 209 | CH═CHCOOCH₃ |
| 210 | CH═CHCOOCH₂CH₃ |
| 211 | CH═CClCOOH |
| 212 | CH═CClCOOCH₃ |
| 213 | CH═CClCOOCH₂CH₃ |
| 214 | CH═CClCOOCH₂CH₂Cl |
| 215 | CH═CClCOOCH₂CH₂OCH₃ |
| 216 | CH═CClCOOCH₂COOCH₃ |
| 217 | CH═CClCONH₂ |
| 218 | CH═CClCONHCH₃ |
| 219 | CH═CClCON(CH₃)₂ |
| 220 | CH═CClCONHCH₂COOCH₃ |
| 221 | CH═CBrCOOH |
| 222 | CH═CBrCOOCH₃ |
| 223 | CH═CBrCOOCH₂CH₃ |
| 224 | CH═CBrCOOCH₂CH₂Cl |
| 225 | CH═CBrCOOCH₂CH₂OCH₃ |
| 226 | CH═CBrCOOCH₂COOCH₃ |
| 227 | CH═C(CH₃)COOH |
| 228 | CH═C(CH₃)COOCH₃ |
| 229 | CH═C(CH₃)COOCH₂CH₃ |
| 230 | CH═C(CH₃)COOCH₂CH₂Cl |
| 231 | CH═C(CH₃)COOCH₂CH₂OCH₃ |
| 232 | CH═C(CH₃)COOCH₂COOCH₃ |

Especially preferred are, moreover, the compounds of the formulae IAb to IAq mentioned hereinbelow, in which the variable X—R⁵ has the abovementioned meanings, in particular the meanings mentioned in in each case one line of Table 1 (compounds IAb.1-IAb.232 to IAq.1-IAq.232).

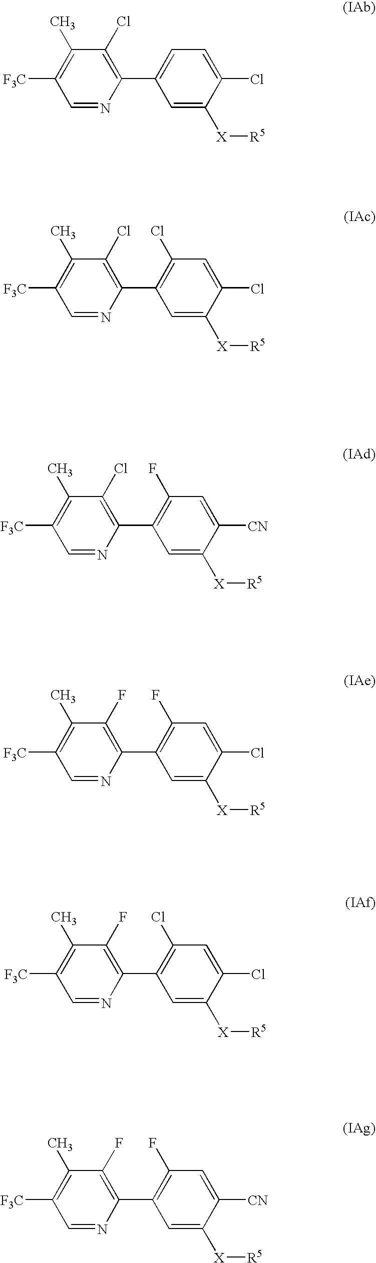

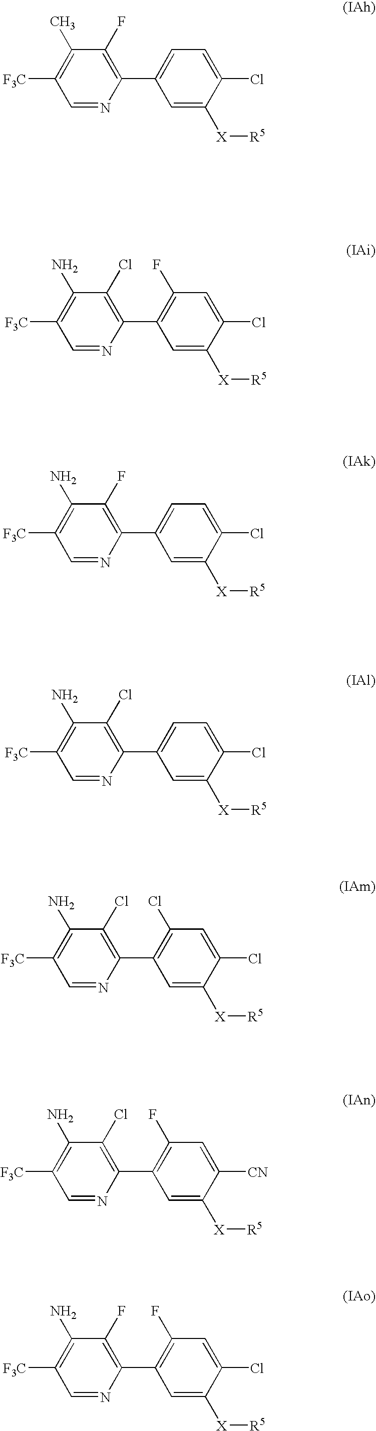

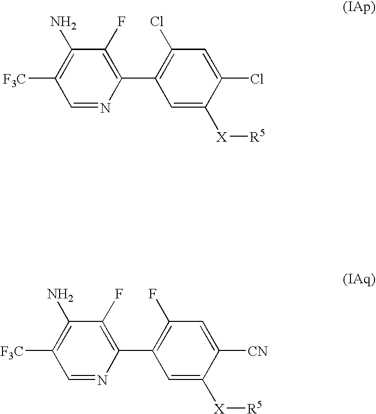

Among the compounds of the formulae IAa to Iaq, the compounds of the formulae IAa to IAh, in particular the compounds of the formulae IAa and Iad, are especially preferred, in particular those where X is a single bond and R⁵ is —CO—O—Y—R⁷ and —O—Y—R⁷ and is especially preferably C₃-C₄-alkynyloxy, OCH(R¹⁹)—COOR²⁰, CO—OR²¹ or COO—CH(R²²)COOR²³, where R⁷, R¹⁹ to R²³ have the abovementioned meanings.

Especially preferred are, moreover, the compounds of the formulae IBa to IBf mentioned hereinbelow in which the variables R³ and R¹⁷ have the abovementioned meanings, in particular the meanings mentioned in in each case one line of Table 2 (compounds IBa.1-IBa.108 to IBf.1-IBf.108).

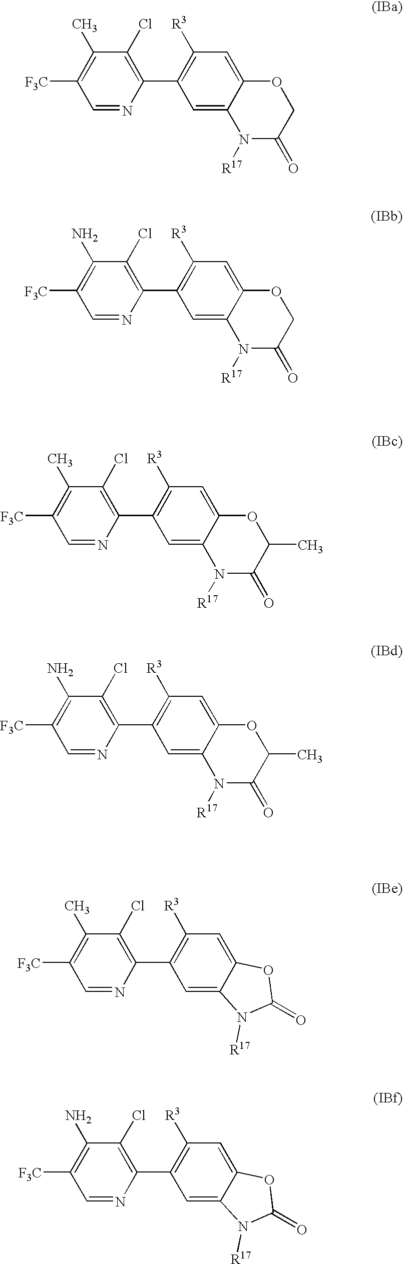

TABLE 2

| No. | R³ | R¹⁷ |
|---|---|---|
| 1 | F | H |
| 2 | F | CH₃ |
| 3 | F | CH₂F |
| 4 | F | CHF₂ |
| 5 | F | CH₂OCH₃ |
| 6 | F | CH₂SCH₃ |
| 7 | F | CH₂—COOCH₃ |
| 8 | F | CH₂—COOC₂H₅ |
| 9 | F | CH(CH₃)—COOCH₃ |
| 10 | F | CH(CH₃)—COOC₂H₅ |
| 11 | F | CH₂CH₃ |
| 12 | F | CH₂CH₂Cl |
| 13 | F | CH₂CH₂OCH₃ |
| 14 | F | CH₂CH₂SCH₃ |
| 15 | F | n—C₃H₇ |
| 16 | F | CH(CH₃)₂ |
| 17 | F | n—C₄H₉ |
| 18 | F | CH(CH₃)—C₂H₅ |
| 19 | F | CH₂—CH(CH₃)₂ |
| 20 | F | CH₂—(cyclo—C₅H₉) |
| 21 | F | CH₂—CH═CH₂ |
| 22 | F | CH₂—CH═CH—CH₃ |
| 23 | F | CH(CH₃)—CH═CH₂ |
| 24 | F | CH₂—C≡CH |
| 25 | F | CH₂—C≡C—CH₃ |
| 26 | F | CH(CH₃)—C≡CH |
| 27 | F | CH₂—phenyl |
| 28 | F | OH |
| 29 | F | O—CH₃ |
| 30 | F | O—CH₂F |
| 31 | F | O—CHF₂ |
| 32 | F | O—CH₂OCH₃ |
| 33 | F | O—CH₂SCH₃ |
| 34 | F | O—CH₂—COOCH₃ |
| 35 | F | O—CH₂—COOC₂H₅ |
| 36 | F | O—CH(CH₃)—COOCH₃ |
| 37 | F | O—CH(CH₃)—COOC₂H₅ |
| 38 | F | O—CH₂CH₃ |
| 39 | F | O—CH₂CH₂Cl |
| 40 | F | O—CH₂CH₂OCH₃ |
| 41 | F | O—CH₂CH₂SCH₃ |
| 42 | F | O—n—C₃H₇ |
| 43 | F | O—CH(CH₃)₂ |
| 44 | F | O—n—C₄H₉ |
| 45 | F | O—CH(CH₃)—C₂H₅ |
| 46 | F | O—CH₂—CH(CH₃)₂ |
| 47 | F | O—CH₂—(cyclo—C₅H₉) |
| 48 | F | O—CH₂—CH═CH₂ |
| 49 | F | O—CH₂—CH═CH—CH₃ |
| 50 | F | O—CH(CH₃)—CH═CH₂ |
| 51 | F | O—CH₂—C≡CH |
| 52 | F | O—CH₂—C≡C—CH₃ |
| 53 | F | O—CH(CH₃)—C≡CH |
| 54 | F | O—CH₂—phenyl |
| 55 | H | H |
| 56 | H | CH₃ |
| 57 | H | CH₂F |
| 58 | H | CHF₂ |
| 59 | H | CH₂OCH₃ |
| 60 | H | CH₂SCH₃ |
| 61 | H | CH₂—COOCH₃ |
| 62 | H | CH₂—COOC₂H₅ |
| 63 | H | CH(CH₃)—COOCH₃ |
| 64 | H | CH(CH₃)—COOC₂H₅ |
| 65 | H | CH₂CH₃ |
| 66 | H | CH₂CH₂Cl |
| 67 | H | CH₂CH₂OCH₃ |
| 68 | H | CH₂CH₂SCH₃ |
| 69 | H | n—C₃H₇ |
| 70 | H | CH(CH₃)₂ |
| 71 | H | n—C₄H₉ |
| 72 | H | CH(CH₃)—C₂H₅ |
| 73 | H | CH₂—CH(CH₃)₂ |
| 74 | H | CH₂—(cyclo—C₅H₉) |
| 75 | H | CH₂—CH═CH₂ |
| 76 | H | CH₂—CH═CH—CH₃ |
| 77 | H | CH(CH₃)—CH═CH₂ |
| 78 | H | CH₂—C≡CH |
| 79 | H | CH₂—C≡C—CH₃ |
| 80 | H | CH(CH₃)—C≡CH |
| 81 | H | CH₂—phenyl |
| 82 | H | OH |
| 83 | H | O—CH₃ |
| 84 | H | O—CH₂F |
| 85 | H | O—CHF₂ |
| 86 | H | O—CH₂OCH₃ |
| 87 | H | O—CH₂SCH₃ |
| 88 | H | O—CH₂—COOCH₃ |
| 89 | H | O—CH₂—COOC₂H₅ |
| 90 | H | O—CH(CH₃)—COOCH₃ |
| 91 | H | O—CH(CH₃)—COOC₂H₅ |
| 92 | H | O—CH₂CH₃ |
| 93 | H | O—CH₂CH₂Cl |
| 94 | H | O—CH₂CH₂OCH₃ |
| 95 | H | O—CH₂CH₂SCH₃ |
| 96 | H | O—n—C₃H₇ |
| 97 | H | O—CH(CH₃)₂ |
| 98 | H | O—n—C₄H₉ |
| 99 | H | O—CH(CH₃)—C₂H₅ |
| 100 | H | O—CH₂—CH(CH₃)₂ |
| 101 | H | O—CH₂—(cyclo—C₅H₉) |
| 102 | H | O—CH₂—CH═CH₂ |
| 103 | H | O—CH₂—CH═CH—CH₃ |
| 104 | H | O—CH(CH₃)—CH═CH₂ |
| 105 | H | O—CH₂—C≡CH |
| 106 | H | O—CH₂—C≡C—CH₃ |
| 107 | H | O—CH(CH₃)—C≡CH |
| 108 | H | O—CH₂—phenyl |

Especially preferred are, moreover, the compounds of the formulae ICa to ICh mentioned hereinbelow in which the variables R³ and R¹⁸ have the abovementioned meaning, in particular the meaning mentioned in in each case one line of Table 3 (compounds ICa.1-ICa.351 to ICh.1-ICh.351).

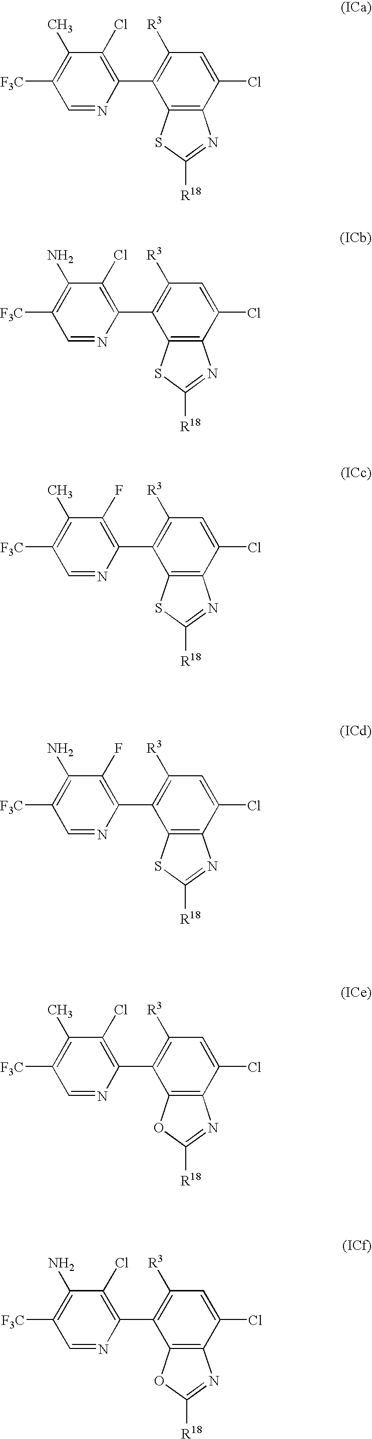

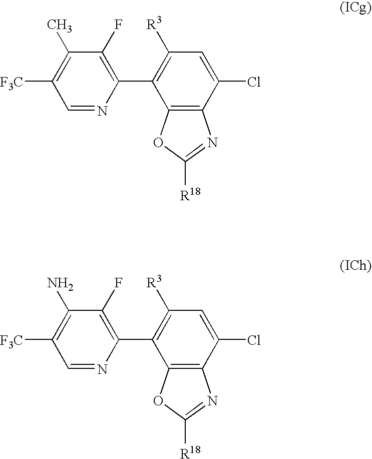

TABLE 3

| No. | R³ | R¹⁸ |
|---|---|---|
| 1 | F | H |
| 2 | F | CH₃ |
| 3 | F | CH₂CH₃ |
| 4 | F | CH₂CH₂CH₃ |
| 5 | F | CH₂CH₂CH₂CH₃ |
| 6 | F | CH(CH₃)₂ |
| 7 | F | CHCH₃CH₂CH₃ |
| 8 | F | C(CH₃)₃ |
| 9 | F | CH₂—cyclopropyl |
| 10 | F | cyclopropyl |
| 11 | F | CH₂CH═CH₂ |
| 12 | F | CH₂CH₂CH═CH₂ |
| 13 | F | CH₂C≡CH |
| 14 | F | CH₂OCH₃ |
| 15 | F | CH₂CH₂OCH₃ |
| 16 | F | CH₂CN |
| 17 | F | CH₂F |
| 18 | F | CH₂Cl |
| 19 | F | CF₃ |
| 20 | F | CH₂COOCH₃ |
| 21 | F | CH₂COOCH₂CH₃ |
| 22 | F | CH₂CON(CH₃)₂ |
| 23 | F | CH₂CH₂CO₂CH₃ |
| 24 | F | CH₂CH₂CO₂CH₂CH₃ |
| 25 | F | CH₂CHClCO₂CH₃ |
| 26 | F | CH₂CHClCOOCH₂CH₃ |
| 27 | F | CH₂CH₂PO(OCH₃)₂ |
| 28 | F | CH₂CH₂PO(OCH₂CH₃)₂ |
| 29 | F | CH₂CHClPO(OCH₃)₂ |
| 30 | F | CH₂CHClPO(OCH₂CH₃)₂ |
| 31 | F | phenyl |
| 32 | F | NO₂ |
| 33 | F | F |
| 34 | F | Cl |
| 35 | F | Br |
| 36 | F | OCH₃ |
| 37 | F | OCH₂CH₃ |
| 38 | F | OCH₂CH₂CH₃ |
| 39 | F | OCH(CH₃)₂ |
| 40 | F | OCH₂CH₂CH₂CH₃ |
| 41 | F | OC(CH₃)₃ |
| 42 | F | OCH₂CH═CH₂ |
| 43 | F | OCH₂CH═CHCH₃ |
| 44 | F | OCH(CH₃)CH═CH₂ |
| 45 | F | OCH₂C≡CH |
| 46 | F | OCH(CH₃)C≡CH |
| 47 | F | OCH₂OCH₃ |
| 48 | F | OCH₂CH₂OCH₃ |
| 49 | F | OCH₂CN |
| 50 | F | OCH₂F |
| 51 | F | OCH₂CF₃ |
| 52 | F | OCH₂COOCH₃ |
| 53 | F | OCH₂COOCH₂CH₃ |
| 54 | F | OCH₂COOCH₂CO₂CH₃ |
| 55 | F | OCH₂CON(CH₃)₂ |
| 56 | F | OCHCH₃COOCH₃ |
| 57 | F | OCHCH₃COOCH₂CH₃ |
| 58 | F | OCH₂COOCH₂COOCH₃ |
| 59 | F | OCH₂PO(OCH₃)₂ |
| 60 | F | OCH₂PO(OCH₂CH₃)₂ |
| 61 | F | OCH₂CH₂PO(OCH₃)₂ |
| 62 | F | OCH₂CH₂PO(OCH₂CH₃)₂ |
| 63 | F | O—phenyl |
| 64 | F | NH₂ |
| 65 | F | NHCH₃ |
| 66 | F | N(CH₃)₂ |
| 67 | F | NHCH₂CH₃ |
| 68 | F | N(CH₂CH₃)₂ |
| 69 | F | NHCH₂CH₂CH₃ |
| 70 | F | N(CH₂CH₂CH₃)₂ |
| 71 | F | NHCH(CH₃)₂ |
| 72 | F | N(CH(CH₃)₂)₂ |
| 73 | F | NHCH₂CH═CH₂ |
| 74 | F | N(CH₂CH═CH₂)₂ |
| 75 | F | NHCH₂CH═CHCH₃ |
| 76 | F | N(CH₂CH═CHCH₃)₂ |
| 77 | F | NHCH₂C≡CH |
| 78 | F | N(CH₂C≡CH)₂ |
| 79 | F | NHCH₂COOCH₃ |
| 80 | F | NHCH₂COOCH₂CH₃ |
| 81 | F | NHCH₂COOCH₂CO₂CH₃ |
| 82 | F | NCH₃CH₂COOCH₃ |
| 83 | F | NCH₃CH₂COOCH₂CH₃ |
| 84 | F | NCH₃CH₂COOCH₂CO₂CH₃ |
| 85 | F | NCH₃CH(CH₃)CO₂CH₃ |
| 86 | F | SH |
| 87 | F | SCH₃ |
| 88 | F | SCH₂CH₃ |
| 89 | F | SCH₂CH₂CH₃ |
| 90 | F | SCH(CH₃)₂ |
| 91 | F | SCH₂CH₂CH₂CH₃ |
| 92 | F | SC(CH₃)₃ |
| 93 | F | SCH₂CH═CH₂ |
| 94 | F | SCH₂CH═CHCH₃ |
| 95 | F | SCH(CH₃)CH═CH₂ |
| 96 | F | SCH₂C≡CH |
| 97 | F | SCH(CH₃)C≡CH |
| 98 | F | SCH₂OCH₃ |
| 99 | F | SCH₂CH₂OCH₃ |
| 100 | F | SCH₂CN |
| 101 | F | SCH₂F |
| 102 | F | SCH₂CF₃ |
| 103 | F | SCH₂COOCH₃ |
| 104 | F | SCH₂COOCH₂CH₃ |
| 105 | F | SCH₂CON(CH₃)₂ |
| 106 | F | SCHCH₃COOCH₃ |
| 107 | F | SCH₂PO(OCH₃)₂ |
| 108 | F | SCH₂PO(OCH₂CH₃)₂ |
| 109 | F | SCH₂CH₂PO(OCH₃)₂ |
| 110 | F | SCH₂CH₂PO(OCH₂CH₃)₂ |
| 111 | F | SCHCH₃COOCH₂CH₃ |
| 112 | F | SCH₂COOCH₂COOCH₃ |
| 113 | F | S—phenyl |
| 114 | F | S(O)CH₃ |
| 115 | F | S(O)CH₂CH₃ |
| 116 | F | S(O)₂CH₃ |
| 117 | F | S(O)₂CH₂CH₃ |
| 118 | Cl | H |
| 119 | Cl | CH₃ |
| 120 | Cl | CH₂CH₃ |
| 121 | Cl | CH₂CH₂CH₃ |
| 122 | Cl | CH₂CH₂CH₂CH₃ |
| 123 | Cl | CH(CH₃)₂ |
| 124 | Cl | CHCH₃CH₂CH₃ |
| 125 | Cl | C(CH₃)₃ |
| 126 | Cl | CH₂—cylopropyl |
| 127 | Cl | cyclopropyl |
| 128 | Cl | CH₂CH═CH₂ |
| 129 | Cl | CH₂CH₂CH═CH₂ |
| 130 | Cl | CH₂C≡CH |
| 131 | Cl | CH₂OCH₃ |
| 132 | Cl | CH₂CH₂OCH₃ |
| 133 | Cl | CH₂CN |
| 134 | Cl | CH₂F |
| 135 | Cl | CH₂Cl |
| 136 | Cl | CF₃ |
| 137 | Cl | CH₂COOCH₃ |
| 138 | Cl | CH₂COOCH₂CH₃ |
| 139 | Cl | CH₂CON(CH₃)₂ |
| 140 | Cl | CH₂CH₂CO₂CH₃ |
| 141 | Cl | CH₂CH₂CO₂CH₂CH₃ |
| 142 | Cl | CH₂CHClCO₂CH₃ |
| 143 | Cl | CH₂CHClCOOCH₂CH₃ |
| 144 | Cl | CH₂CH₂PO(OCH₃)₂ |
| 145 | Cl | CH₂CH₂PO(OCH₂CH₃)₂ |
| 146 | Cl | CH₂CHClPO(OCH₃)₂ |
| 147 | Cl | CH₂CHClPO(OCH₂CH₃)₂ |
| 148 | Cl | phenyl |
| 149 | Cl | NO₂ |
| 150 | Cl | F |
| 151 | Cl | Cl |
| 152 | Cl | Br |
| 153 | Cl | OCH₃ |
| 154 | Cl | OCH₂CH₃ |
| 155 | Cl | OCH₂CH₂CH₃ |
| 156 | Cl | OCH(CH₃)₂ |
| 157 | Cl | OCH₂CH₂CH₂CH₃ |
| 158 | Cl | OC(CH₃)₃ |
| 159 | Cl | OCH₂CH═CH₂ |
| 160 | Cl | OCH₂CH═CHCH₃ |
| 161 | Cl | OCH(CH₃)CH═CH₂ |
| 162 | Cl | OCH₂C≡CH |
| 163 | Cl | OCH(CH₃)C≡CH |
| 164 | Cl | OCH₂OCH₃ |
| 165 | Cl | OCH₂CH₂OCH₃ |
| 166 | Cl | OCH₂CN |
| 167 | Cl | OCH₂F |
| 168 | Cl | OCH₂CF₃ |
| 169 | Cl | OCH₂COOCH₃ |
| 170 | Cl | OCH₂COOCH₂CH₃ |
| 171 | Cl | OCH₂COOCH₂CO₂CH₃ |
| 172 | Cl | OCH₂CON(CH₃)₂ |
| 173 | Cl | OCHCH₃COOCH₃ |
| 174 | Cl | OCHCH₃COOCH₂CH₃ |
| 175 | Cl | OCH₂COOCH₂COOCH₃ |
| 176 | Cl | OCH₂PO(OCH₃)₂ |
| 177 | Cl | OCH₂PO(OCH₂CH₃)₂ |
| 178 | Cl | OCH₂CH₂PO(OCH₃)₂ |
| 179 | Cl | OCH₂CH₂PO(OCH₂CH₃)₂ |
| 180 | Cl | O—phenyl |
| 181 | Cl | NH₂ |
| 182 | Cl | NHCH₃ |
| 183 | Cl | N(CH₃)₂ |
| 184 | Cl | NHCH₂CH₃ |
| 185 | Cl | N(CH₂CH₃)₂ |
| 186 | Cl | NHCH₂CH₂CH₃ |
| 187 | Cl | N(CH₂CH₂CH₃)₂ |
| 188 | Cl | NHCH(CH₃)₂ |
| 189 | Cl | N(CH(CH₃)₂)₂ |
| 190 | Cl | NHCH₂CH═CH₂ |
| 191 | Cl | N(CH₂CH═CH₂)₂ |
| 192 | Cl | NHCH₂CH═CHCH₃ |
| 193 | Cl | N(CH₂CH═CHCH₃)₂ |
| 194 | Cl | NHCH₂C≡CH |
| 195 | Cl | N(CH₂C≡CH)₂ |
| 196 | Cl | NHCH₂COOCH₃ |
| 197 | Cl | NHCH₂COOCH₂CH₃ |
| 198 | Cl | NHCH₂COOCH₂CO₂CH₃ |
| 199 | Cl | NCH₃CH₂COOCH₃ |
| 200 | Cl | NCH₃CH₂COOCH₂CH₃ |
| 201 | Cl | NCH₃CH₂COOCH₂CO₂CH₃ |
| 202 | Cl | NCH₃CH(CH₃)CO₂CH₃ |
| 203 | Cl | SH |
| 204 | Cl | SCH₃ |
| 205 | Cl | SCH₂CH₃ |
| 206 | Cl | SCH₂CH₂CH₃ |
| 207 | Cl | SCH(CH₃)₂ |
| 208 | Cl | SCH₂CH₂CH₂CH₃ |
| 209 | Cl | SC(CH₃)₃ |
| 210 | Cl | SCH₂CH═CH₂ |
| 211 | Cl | SCH₂CH═CHCH₃ |
| 212 | Cl | SCH(CH₃)CH═CH₂ |
| 213 | Cl | SCH₂C≡CH |
| 214 | Cl | SCH(CH₃)C≡CH |
| 215 | Cl | SCH₂OCH₃ |
| 216 | Cl | SCH₂CH₂OCH₃ |
| 217 | Cl | SCH₂CN |
| 218 | Cl | SCH₂F |
| 219 | Cl | SCH₂CF₃ |
| 220 | Cl | SCH₂COOCH₃ |
| 221 | Cl | SCH₂COOCH₂CH₃ |
| 222 | Cl | SCH₂CON(CH₃)₂ |
| 223 | Cl | SCHCH₃COOCH₃ |
| 224 | Cl | SCH₂PO(OCH₃)₂ |
| 225 | Cl | SCH₂PO(OCH₂CH₃)₂ |
| 226 | Cl | SCH₂CH₂PO(OCH₃)₂ |
| 227 | Cl | SCH₂CH₂PO(OCH₂CH₃)₂ |
| 228 | Cl | SCHCH₃COOCH₂CH₃ |
| 229 | Cl | SCH₂COOCH₂COOCH₃ |
| 230 | Cl | S—phenyl |
| 231 | Cl | S(O)CH₃ |
| 232 | Cl | S(O)CH₂CH₃ |
| 233 | Cl | S(O)₂CH₃ |
| 234 | Cl | S(O)₂CH₂CH₃ |
| 235 | H | H |
| 236 | H | CH₃ |
| 237 | H | CH₂CH₃ |
| 238 | H | CH₂CH₂CH₃ |
| 239 | H | CH₂CH₂CH₂CH₃ |
| 240 | H | CH(CH₃)₂ |
| 241 | H | CHCH₃CH₂CH₃ |
| 242 | H | C(CH₃)₃ |
| 243 | H | CH₂—cyclopropyl |
| 244 | H | cyclopropyl |
| 245 | H | CH₂CH═CH₂ |
| 246 | H | CH₂CH₂CH═CH₂ |
| 247 | H | CH₂C≡CH |
| 248 | H | CH₂OCH₃ |
| 249 | H | CH₂CH₂OCH₃ |
| 250 | H | CH₂CN |
| 251 | H | CH₂F |
| 252 | H | CH₂Cl |
| 253 | H | CF₃ |
| 254 | H | CH₂COOCH₃ |
| 255 | H | CH₂COOCH₂CH₃ |
| 256 | H | CH₂CON(CH₃)₂ |
| 257 | H | CH₂CH₂CO₂CH₃ |
| 258 | H | CH₂CH₂CO₂CH₂CH₃ |
| 259 | H | CH₂CHClCO₂CH₃ |
| 260 | H | CH₂CHClCOOCH₂CH₃ |
| 261 | H | CH₂CH₂PO(OCH₃)₂ |
| 262 | H | CH₂CH₂PO(OCH₂CH₃)₂ |
| 263 | H | CH₂CHClPO(OCH₃)₂ |
| 264 | H | CH₂CHClPO(OCH₂CH₃)₂ |
| 265 | H | phenyl |
| 266 | H | NO₂ |
| 267 | H | F |
| 268 | H | Cl |
| 269 | H | Br |
| 270 | H | OCH₃ |
| 271 | H | OCH₂CH₃ |
| 272 | H | OCH₂CH₂CH₃ |
| 273 | H | OCH(CH₃)₂ |
| 274 | H | OCH₂CH₂CH₂CH₃ |
| 275 | H | OC(CH₃)₃ |
| 276 | H | OCH₂CH═CH₂ |
| 277 | H | OCH₂CH═CHCH₃ |
| 278 | H | OCH(CH₃)CH═CH₂ |
| 279 | H | OCH₂C≡CH |
| 280 | H | OCH(CH₃)C≡CH |
| 281 | H | OCH₂OCH₃ |
| 282 | H | OCH₂CH₂OCH₃ |
| 283 | H | OCH₂CN |
| 284 | H | OCH₂F |
| 285 | H | OCH₂CF₃ |
| 286 | H | OCH₂COOCH₃ |
| 287 | H | OCH₂COOCH₂CH₃ |
| 288 | H | OCH₂COOCH₂CO2CH₃ |
| 289 | H | OCH₂CON(CH₃)₂ |
| 290 | H | OCHCH₃COOCH₃ |
| 291 | H | OCHCH₃COOCH₂CH₃ |
| 292 | H | OCH₂COOCH₂COOCH₃ |
| 293 | H | OCH₂PO(OCH₃)₂ |
| 294 | H | OCH₂PO(OCH₂CH₃)₂ |
| 295 | H | OCH₂CH₂PO(OCH₃)₂ |
| 296 | H | OCH₂CH₂PO(OCH₂CH₃)₂ |
| 297 | H | O—phenyl |
| 298 | H | NH₂ |
| 299 | H | NHCH₃ |
| 300 | H | N(CH₃)₂ |
| 301 | H | NHCH₂CH₃ |
| 302 | H | N(CH₂CH₃)₂ |
| 303 | H | NHCH₂CH₂CH₃ |
| 304 | H | N(CH₂CH₂CH₃)₂ |
| 305 | H | NHCH(CH₃)₂ |
| 306 | H | N(CH(CH₃)₂)₂ |
| 307 | H | NHCH₂CH═CH₂ |
| 308 | H | N(CH₂CH═CH₂)₂ |
| 309 | H | NHCH₂CH═CHCH₃ |
| 310 | H | N(CH₂CH═CHCH₃)₂ |
| 311 | H | NHCH₂C≡CH |
| 312 | H | N(CH₂C≡CH)₂ |
| 313 | H | NHCH₂COOCH₃ |
| 314 | H | NHCH₂COOCH₂CH₃ |
| 315 | H | NHCH₂COOCH₂CO₂CH₃ |
| 316 | H | NCH₃CH₂COOCH₃ |
| 317 | H | NCH₃CH₂COOCH₂CH₃ |
| 318 | H | NCH₃CH₂COOCH₂CO₂CH₃ |
| 319 | H | N(CH₃)CH(CH₃)CO₂CH₃ |
| 320 | H | SH |
| 321 | H | SCH₃ |
| 322 | H | SCH₂CH₃ |
| 323 | H | SCH₂CH₂CH₃ |
| 324 | H | SCH(CH₃)₂ |
| 325 | H | SCH₂CH₂CH₂CH₃ |
| 326 | H | SC(CH₃)₃ |
| 327 | H | SCH₂CH═CH₂ |
| 328 | H | SCH₂CH═CHCH₃ |
| 329 | H | SCH(CH₃)CH═CH₂ |
| 330 | H | SCH₂C≡CH |
| 331 | H | SCH(CH₃)C≡CH |
| 332 | H | SCH₂OCH₃ |
| 333 | H | SCH₂CH₂OCH₃ |
| 334 | H | SCH₂CN |
| 335 | H | SCH₂F |
| 336 | H | SCH₂CF₃ |
| 337 | H | SCH₂COOCH₃ |
| 338 | H | SCH₂COOCH₂CH₃ |
| 339 | H | SCH₂CON(CH₃)₂ |
| 340 | H | SCHCH₃COOCH₃ |
| 341 | H | SCH₂PO(OCH₃)₂ |
| 342 | H | SCH₂PO(OCH₂CH₃)₂ |
| 343 | H | SCH₂CH₂PO(OCH₃)₂ |
| 344 | H | SCH₂CH₂PO(OCH₂CH₃)₂ |
| 345 | H | SCHCH₃COOCH₂CH₃ |
| 346 | H | SCH₂COOCH₂COOCH₃ |
| 347 | H | S—phenyl |
| 348 | H | S(O)CH₃ |
| 349 | H | S(O)CH₂CH₃ |
| 350 | H | S(O)₂CH₃ |
| 351 | H | S(O)₂CH₂CH₃ |

The 2-aryl-5-trifluoromethylpyridines, their N-oxides and their salts can be prepared analogously to the preparation of the 2-aryl-5-trifluoromethylpyridines, which are known from the prior 5 art cited at the outset.

Preferred procedure for this purpose is to couple a suitably substituted pyridine of the formula II

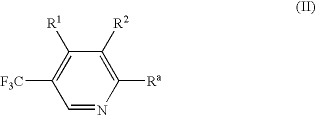

in which the variables R¹ and R² have the abovementioned meanings, or R¹ is a protected amino group, and

R^{a} is halogen or S(O)ₖ-phenyl where k is 0, 1 or 2

with an organometallic compound of the formula III

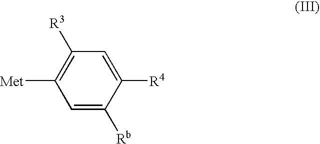

where Met is a metal atom or a semimetal or a radical bonded via a metal atom or semimetal atom, R³ and R⁴ have the abovementioned meanings and R^{b} is a substituent which is compatible with the metal atom or the semimetal which can be converted by known methods into one of the groups X—R⁵, or is a group X—R⁵ which is compatible with the metal or the semimetal. The reaction of II with III is preferably carried out in the presence of catalytically active amounts of a transition metal of the VIIIb group of the periodic system, for example Ni or Pd, it being possible for the metal to be employed as such, in doped or supported form, as a complex compound or as a salt.

Examples of suitable groups Met are, in particular, Mg-Hal and Zn-Hal, where Hal is halogen, and —B(OR′)₂, where R′ is hydrogen or C₁-C₁₀-alkyl.

Examples of suitable radicals R^{b} are the groups X—R^{5′} mentioned hereinbelow, where X has the abovementioned meanings and R^{5′} is selected from among hydrogen, cyano, halogen, —O—Y—R⁷, —O—CO—Y—R⁷, —N(Y—R⁷)(Z-R⁸), —S—Y—R⁷, —CO—Y—R⁷, —CO—O—Y—R⁷, —CO—N(Y—R⁷)(Z-R⁸), —CO—N(Y—R⁷)(O-Z-R⁸) and —PO(O—Y—R⁷)₂; with the abovementioned meanings of Y, Z, R⁷ and R⁸. R^{b} is, in particular, hydrogen, C₁-C₄-alkyl, halogen, a group —O—Y—R⁷ or a group —CO—O—Y—R⁷.

The above-defined pyridines of the formula II where R^{a} is OH, C₁-C₄-alkoxy or benzyloxy (compounds VI, VII, IX and X described hereinbelow and N-protected derivatives of II where R¹═NH₂) and the boronic acid compounds of the formula IIIa

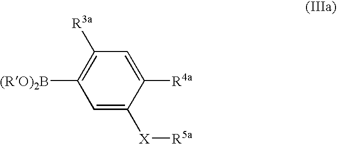

in which X is a single bond and the variables R′, R^{3a}, R^{4a} and R^{5a} have the following meanings:

R′ is hydrogen or C₁-C₁₀-alkyl or two radicals R′ form a chain of the formula —CH₂—CH₂— or —CH₂—CH₂—CH₂;

R^{3a} is hydrogen or halogen;

R^{4a} is halogen or C₁-C₄-alkoxy;

R^{5a} is hydrogen, cyano, halogen, —O—Y—R^{7a}, —O—CO—Y—R⁷, —S—Y—R^{7a}, —CO—O—Y—R⁷ or —PO(O—Y—R^{7a})₂; where R^{7a} is a group —C(R¹⁰)(R¹¹)—CO—OR¹² and Y, R⁷, R¹⁰, R¹¹ and R¹² have the abovementioned meanings;

or R^{4a} is CN and R^{5a} has the following meaning:

R^{5a} is cyano, halogen, —O—Y—R⁷, —O—CO—Y—R⁷, —S—Y—R⁷, —CO—O—Y—R⁷ or —PO(O—Y—R⁷)₂; where Y and R⁷ have the abovementioned meanings;

are novel and, being important intermediates for the preparation of the 2-aryl-5-trifluoromethylpyridines of the formula I according to the invention, are likewise subject matter of the present invention. In the boronic acids IIIa, X and Y are preferably single bonds. Especially important intermediates among the boronic acid derivatives IIIa are those compounds in which R^{4a} is chlorine and X—R^{5a} is CN, —O—Y—R^{7a}, —O—CO—Y—R⁷ or —CO—O—Y—R⁷. In these formulae, R⁷ has the abovementioned meanings and in this case is especially preferably C₁-C₄-alkyl or C₁-C₄-alkyloxycarbonyl-C₁-C₄-alkyl. R^{7a} is preferably a C₁-C₄-alkyloxycarbonyl-C₁-C₄-alkyl radical. If R^{4a} is CN, then X—R^{5a} is preferably cyano, halogen, —O—Y—R⁷, —O—CO—Y—R⁷ or —CO—O—Y—R⁷. R⁷ in this case preferably represents C₁-C₄-alkyl or C₁-C₄-alkyloxycarbonyl-C₁-C₄-alkyl.

To prepare the compounds I according to the invention, it is preferred to react a chloropyridine derivative (compound II where R^{a}═Cl) with a phenylboronic acid or boronic acid ester (compound III where Met=B(OH)₂ or B(OR′)₂) or with a Grignard compound (compound III where Met=Hal-Mg, for example Cl—Mg) or with a zinc compound (compound III where Met=Hal-Zn, in particular Cl—Zn) in the presence of catalytically active amounts of a palladium or nickel compound and in the event of boronic acid coupling additionally in the presence of a base in an organic solvent or in a mixture of an organic solvent with water at ambient temperature or elevated temperatures.

The processes and conditions for such reactions are known to the skilled worker and can be found for example in the reviews by F. Diederich, P. J. Stang (Ed.) Metal-catalyzed Cross-coupling Reactions, Wiley-VCH-Verlag Weinheim 1998, W. A. Herrmann et al., Angew. Chem. 39, 2000, p. 1602, or W. A. Herrmann et al., “Applied Homogeneous Catalysis with Organometallic Compounds” Wiley-VCH 1996, p. 764, and in WO 95/02580, WO 95/02590, WO 98/11070, EP 972765-A1 and the prior art stated therein.

Suitable palladium catalyts are, in addition to palladium carboxylates such as palladium(II) acetate, also palladium/phosphane complexes such as tetrakistriphenylphosphanepalladium, totriphenylphosphanepalladium(II) chloride, to (1,2-diphenylphosphanoethane)palladium(II) chloride, to (1,3-diphenylphosphanopropane)palladium(II) chloride, to (1,4-diphenylphosphanobutane)palladium(II) chloride and to (diphenylphosphano)ferrocenylpalladium(II) chloride. However, palladium halides such as palladium(II) chloride may also be reacted in situ with phosphine ligands to give the catalytically active complexes. Examples of suitable phosphine ligands are arylphosphanes which are unsubstituted or substituted in the ortho, meta or para position by halogen, alkyl and/or SO₃H, such as triphenylphosphine, 1,2-bis(diphenylphosphano)ethane, 1,3-bis(diphenylphosphano)propane, 1,4-bis(diphenylphosphano)butane, to (diphenylphosphano)ferrocene, hetarylphosphanes such as trisfurylphosphine or trispyridylphosphine. Corresponding platinum catalysts are also suitable.

Suitable Ni catalysts are nickel(II) acetyl acetonate, alone or in conjunction with the abovementioned phosphine ligands, or Ni(II) acetyl acetonate with imidazolium carbene ligands, and complexes of nickel(II) salts with the abovementioned phosphine ligands, for example to (triphenylphosphine)nickel(II) chloride, [1,3-bis(diphenylphosphano)propane]nickel(II) chloride, [1,4-bis(diphenylphosphano)butane]nickel(II) chloride and [bis(diphenylphosphano)ferrocene]nickel(II) chloride.

The catalyst is usually employed in a substoichiometric amount, preferably from 0.001-0.8 equivalents and especially preferably from 0.01 to 0.5 equivalents, based on the pyridine II employed.

The molar ratio of compound II to compound III is preferably in the range of from 0.95:1 to 1:1.5.

If required, suitable bases are alkali metal hydroxides, alkaline earth metal hydroxides, alkali metal (hydrogen) carbonates and alkali metal (hydrogen) phosphates such as NaOH, NaHCO₃, Na₂CO₃, KHCO₃, K₂CO₃, Ba(OH)₂, K₃PO₄, alkali metal alkoxides, alkaline earth metal alkoxides, thallium alkoxides and transition metal alkoxides such as sodium ethoxide and thallium ethoxide. Alkali metal fluorides such as potassium fluoride, cesium fluoride, ammonium fluorides and tetrabutylammonium fluoride are also suitable as bases. The base is usually employed in an approximately stoichiometric amount or in an up to 10-fold excess, based on compound II.

Suitable solvents are organic solvents such as DMF, dimethylacetamide, toluene, tetrahydrofuran (THF), dioxane and dimethoxyethane. In the event of boronic acid coupling, the abovementioned solvents may also be employed in a mixture with water, for example in a ratio of approximately 5:1 to 1:5, preferably in a ratio of approximately 2:1 to 1:2, and in particular of approximately 1:1.

The reaction temperature is usually above the melting point and can be up to the boiling point of the solvent. It is preferably in the range between 50 and 150° C.

Moreover, the compounds I according to the invention can also be obtained by coupling the corresponding 2-pyridinyl sulfoxides (compounds II where R^{a}═S(O)ₖR^{b}) or 2-pyridinyl sulfones (compounds II where R^{a}═S(O)₂R^{b}) with a phenyl-Grignard compound III (compound III where Met=Mg-Hal). The reaction can be carried out analogously to the procedures described in JP 2000080082, WO 98 54137, WO 98 11069, WO 98/11070 and WO 98/11072, so that reference is made herewith to the disclosure of these publications.

The compounds of the formula II which are required for the preparation of the 2-aryl-5-trifluoromethylpyridines I according to the invention can be prepared starting from the commercially available dichloropyridines IV (Hal=Cl, R^{2d}═Cl, CAS-No.:69045-84-7, Hal=R^{2d}═F, CAS-No.: 89402-42-6) following the schemes 1 and 2 hereinbelow.

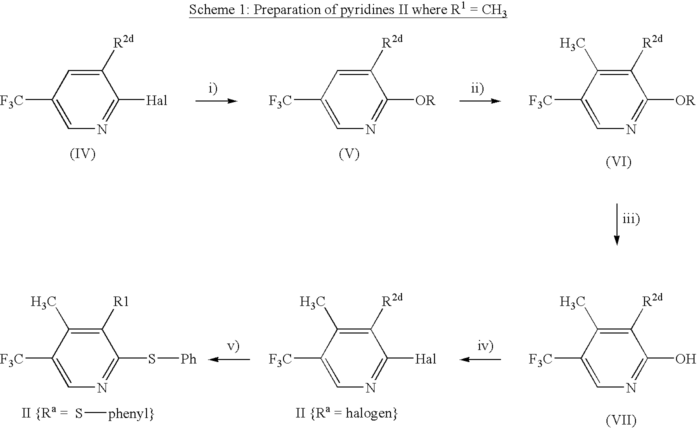

In scheme 1, R^{2d} is halogen, in particular fluorine or chlorine. Hal is also halogen, in particular fluorine or chlorine. R is C₁-C₁₀-alkyl or benzyl. R^{b} has the abovementioned meanings.

In accordance with scheme 1, the pyridine compounds V are first prepared by reacting the dihalopyridines IV with alcohols ROH in the presence of bases or by reacting IV with the corresponding alkoxides (step i)). Such reactions are known in principle and described, for example, in Tome et al. Tetrahedron Lett. 34 (41) 1993 p. 6639, Gerster et al. J. Org. Chem. 31 1966 p. 3259 and in WO 98/11069, which are herewith referred to.

Surprisingly, the introduction of the methyl group in the 4-position of the pyridine ring in step ii) can be carried out by a two-step reaction sequence comprising first the metalation, in particular lithiation, of the 4-position and subsequently the reaction of the pyridine anion thus obtained with an electrophilic methylating agent. An undesired halogen-metal exchange or the formation of undesired isomers or adducts in the 6-position is not observed. This procedure opens up for the first time a route for the preparation of the compounds II and thus for the preparation of the compounds I. The compounds II and the methods illustrated in schemes 1 and 2 are therefore also subject matter of the present invention.

This procedure has not been described as yet in the prior art, even though examples of regioselective metalations on pyridine derivatives are found occasionally in the literature. EP-A 0953566, for example, describes the derivatization of 2-alkoxy-5-trifluoromethylpyridines by metalation of the 4-position of the pyridine ring in the vicinity of a trifluoromethyl group using sterically demanding lithium amide bases. However, this publication does not teach that such a metalation is possible in pyridines which have a halogen atom bonded to the pyridine ring without a halogen-metal exchange taking place. In accordance with the prior art, this would have been expected owing to the ortho-directing effect of the alkoxy group (see JOC 1990, 55 p. 69).

To carry out the lithiation, the pyridine derivative V is usually reacted, in step ii), with at least one equivalent of an organolithium compoumd, for methyllithium, n-butyllithium or sec-butyllithium, or with a lithium amide such as lithium diisopropylamide or lithium-2,2,6,6-tetramethylpiperidine (LiTMP) in an aprotic, preferably etherial, organic solvent such as tetrahydrofuran or methyl tert-butyl ether. As a rule, the reaction is carried out at temperatures of below −30° C., preferably in the range of −120° C. to −40° C., and in particular in the range of from −75° C. to −60° C. To carry out the methylation, 1 to 20 equivalents, preferably 1 to 10 equivalents, of an electrophilic methylating agent are subsequently added. In some cases it may be advantageous to add the lithiated pyridine to a solution of electrophilic methylating agent.

Suitable as electrophilic methylating agents are a multiplicity of customary methylating agents such as methyl halides, preferably methyl chloride, methyl bromide, methyl iodide, furthermore dimethyl sulfate, methyl tosylate and methyl triflate.

Starting with the 2-alkoxy-4-methyl-5-trifluoromethylpyridines VI obtained in step ii), the halopyridines II are then prepared in a two-step synthesis sequence comprising an ether cleavage of the pyridine IV in step iii) and the subsequent conversion of the resulting hydroxypyridine VII or the tautomeric pyridone in step iv) into the halogen compound, in particular into the chlorine compound II {R^{a}=halogen, in particular chlorine}.

To carry out the ether cleavage in step iii), the pyridine compound VI is treated with a strong Lewis acid such as, for example, boron tribromide, trimethylsilyl iodide or a hydrohalic acid such as concentrated hydrobromic acid, depending on the radical R. If R in formula VI is benzyl, the ether cleavage can also be carried out by means of hydrogenolysis, for example by treating VI with hydrogen in the presence of a transition metal catalyst such as palladium or platinum on active charcoal or Raney nickel. The conditions for this procedure follow the methods known from protection-group chemistry as are described, for example, in Kocienski et al. “Protecting Groups”, Thieme Verlag 1994.

The subsequent conversion of the hydroxypyridine VII in step iv), which, depending on the solvent, may also be present in the form of the tautomeric pyridone, is known to the skilled worker in principle and is generally carried out by reacting VII with a Lewis-acidic halogenating agent such as phosgene, thionyl chloride, phosphorus oxychloride or phosphorus(V) chloride. To this end, the halogenating agent is employed in equimolar amounts or in an up to 10-fold excess in an inert organic solvent such as chloroform, dichloroethane, toluene or in very large excess as the solvent. As a rule, the reaction temperatures range from 20° C. to 120° C., preferably from 40° C. to 100° C., very especially preferably from 40° C. to 80° C. As regards further details on steps iii) and iv), reference is made at this point to EP-A 72777, in particular the examples, which apply analogously to steps iii) and iv) of Scheme 1.

The thiopyridines II can then be prepared analogously to processes known from the literature by reacting chloropyridines II with thiols R^{b}SH in the presence of a base or of a catalyst. As regards the reaction conditions for these reactions, reference is made to WO 98/11072, WO 98/11070, WO 98/11069 and WO 98/54137, WO 98/54139 and JP 2000080082. The further oxidation to give the sulfoxides II {R^{a}═SO-phenyl} or the sulfones II {R^{a}═SO₂-phenyl} can also be carried out analogously to the publications mentioned herein.

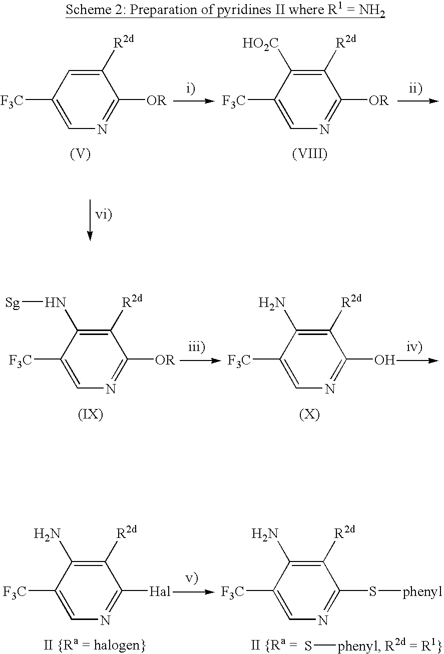

In Scheme 2, R^{2d} is halogen, in particular fluorine or chlorine. Hal is also halogen, in particular fluorine or chlorine. R is C₁-C₁₀-alkyl or benzyl. R^{b} has the abovementioned meanings. Sg is hydrogen or a protecting group.

The preparation of the aminopyridines II (R¹═NH₂) is similar to the preparation of the methylpyridines II (R¹═CH₃). First, an alkoxypyridine compound V is metalated, in particular lithiated, in step i) and subsequently reacted with CO₂ or a carbonic acid derivative to give the carboxylic acid VIII. As regards step i), what has been said for step ii) in Scheme 1 applies analogously.

Using known processes, the carboxylic acid VIII is then converted in step ii) into the amine IX (Sg=H) or a suitably protected derivative IX. The methods of converting carboxylic acid derivatives into amines are known to the skilled worker as Hofmann, Curtius and Schmidt degradation. As regards the conditions for the reaction, reference is made for example to Houben-Weyl Organo-Stickstoff-Verbindungen IV, Vol. E16d Part 2, pages 1160-1167, Thieme Verlag Stuttgart.

Steps iii), iv) and v) of Scheme 2 are then carried out analogously to the steps described in Scheme 1. If Sg is a protecting group, that is to say other than hydrogen, Sg is generally eliminated under ether cleavage conditions (step iii) in Schemes 1 and 2). When oxidizing the aminomercaptopyridines II (R¹═NH₂, R^{a}═S—R^{b}), it may be necessary to introduce a protecting group at the amino group before the oxidation. Suitable protecting groups are, for example, acetyl and benzyloxycarbonyl.

Moreover, the aminopyridines II can be prepared by lithiating compound V and subsequently reacting the lithiated pyridine with an electrophilic aminating reagent such as tosyl azide, phosphinyl azide, t-butylvinyl azide, hydroxylamine or 2,4-dinitrophenyl hydroxylamine ether (Scheme 2, step vi)). These methods are known to the skilled worker and described, for example, in K. Krohn, Electrophilic Amination, in Mulzer, Altenbach, Braun, Krohn, Reissig (editors) “Organic Synthesis Highlights” VCH 1991, p. 45; Kozikowski et al. Tetrahedron Lett. 30 (33) 1989, p. 4613.

TABLE 4a
Intermediates of Scheme 1

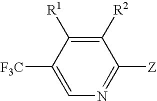

| Compound | R¹ | R² | Z |
|---|---|---|---|
| V.1 | H | Cl | OCH₂Ph |
| V.2 | H | Cl | OCH₃ |
| V.3 | H | Cl | OCH₂CH₃ |
| V.4 | H | Cl | OCH(CH₃)2 |
| V.5 | H | F | OCH₂Ph |
| V.6 | H | F | OCH₃ |
| V.7 | H | F | OCH₂CH₃ |
| V.8 | H | F | OCH(CH₃)₂ |
| VI.1 | CH₃ | Cl | OCH₂Ph |
| VI.2 | CH₃ | Cl | OCH₃ |
| VI.3 | CH₃ | Cl | OCH₂CH₃ |
| VI.4 | CH₃ | Cl | OCH(CH₃)₂ |
| VII.1 | CH₃ | Cl | OH |
| II.1 | CH₃ | Cl | Cl |
| VI.5 | CH₃ | F | OCH₂Ph |
| VI.6 | CH₃ | F | OCH₃ |
| VI.7 | CH₃ | F | OCH₂CH₃ |
| VI.8 | CH₃ | F | OCH(CH₃)₂ |
| VII.2 | CH₃ | F | OH |
| II.2 | CH₃ | F | Cl |
| II.3 | CH₃ | Cl | S-phenyl |
| II.4 | CH₃ | F | S-phenyl |
| II.5 | CH₃ | Cl | S(O)-phenyl |
| II.6 | CH₃ | F | S(O)-phenyl |
| II.7 | CH₃ | Cl | S(O)₂-phenyl |
| II.8 | CH₃ | F | S(O)₂-phenyl |

TABLE 4b
Intermediates of Scheme 2

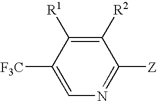

| Compound | R¹ | R² | Z |
|---|---|---|---|
| VIII.1 | COOH | Cl | OCH₂Ph |
| VIII.2 | COOH | Cl | OCH₂Ph |
| IX.1 | tert-butoxycarbonyl-NH | Cl | OCH₂Ph |
| IX.2 | tert-butoxycarbonyl-NH | Cl | OCH₂Ph |
| X.1 | NH₂ | F | OH |
| X.2 | NH₂ | F | OH |
| II.9 | NH₂ | Cl | Cl |
| II.10 | NH₂ | F | Cl |
| II.11 | NH₂ | Cl | S-phenyl |
| II.12 | NH₂ | F | S-phenyl |
| II.13 | NH₂ | Cl | S(O)-phenyl |
| II.14 | NH₂ | F | S(O)-phenyl |
| II.15 | NH₂ | Cl | S(O)₂-phenyl |
| II.16 | NH₂ | F | S(O)₂-phenyl |

Some of the compounds III required for synthesizing the 2-aryl-5-trifluoromethylpyridines I are known from the literature or can generally be prepared by known methods, preferably from the corresponding halogen compounds.

Some of the boronic acids which are especially suitable for preparing the 2-aryl-5-trifluoromethylpyridines I according to the invention (compounds III where Met=B(OR′)₂) are known from the literature, for example 2-fluoro-4-chloro-5-methoxyphenylboronic acid (CAS-No.: 153122-60-2), 2-fluoro-4-chlorophenylboronic acid (CAS-No.: 160591-91-3) or 2-fluoro-4-methoxyphenylboronic acid (CAS-No.: 162101-31-7).

Moreover, they can be prepared analogously to known methods by reacting the corresponding phenyl-Grignard compounds (compound III where Met=Mg-Hal) with boric esters (see, for example, Houben-Weyl, Vol. 13, Part 3a, pages 616-654, Thieme Verlag 1982).

Usually, the preparation of the phenyl-Grignard compounds required for this purpose is carried out as described therein, starting from the corresponding phenyl bromide, and reacting it with magnesium or a second Grignard reagent. The reaction temperatures required for this purpose only make it possible to obtain those Grignard compounds in which the group R⁴ or R^{b} in formula III is a radical which does not react with a Grignard compound.

Surprisingly, it has been found that phenyl-Grignard compounds III (compounds III where Met=Mg-Hal) can be scavenged at low temperatures using borates (R′O)₃B. To this end, the corresponding phenyl iodides are first converted into Grignard compounds. The reaction of functionalized aromatic iodides to give Grignard reagents is known, in principle, from the literature (see, for example, Knochel et al, Angew. Chem. 1998, 110, p. 1801 and DE-A 19836408) and is usually carried out by reacting the phenyl iodides with other Grignard compounds. The conversion of the resulting phenyl-Grignard compounds III into the boronic acids IIIa is then carried out at low temperatures, i.e. below 0° C., in particular at −10° C. and below, that is to say at temperatures at which a series of groups which are reactive toward Grignard compounds, such as carboxylate, amide and nitrile groups, are not yet attacked. Thus, in this manner, even those boronic compounds IIIa (compound IIIa where Met=B(OR′)₂) which have a substituent which is reactive toward Grignard compounds can be prepared for the first time. Accordingly, the present invention also relates to the above-defined phenylboronic acid compounds of the formula IIIa. Depending on work-up and storage, these compounds can either exist only as monomeric boronic acids or as its trimer boroxine or else as mixtures and employed in the reactions described at the outset.

To prepare the boronic acid compounds (compound III where Met=B(OR′)₂), the corresponding iodides are first converted into the corresponding phenyl-Grignard compound by means of another Grignard compound. Suitable for this purpose are, in particular, alkyl Grignard compounds, for example C₁-C₄-alkylmagnesium halides, in particular the bromides such as methylmagnesium bromide or isopropylmagnesium bromide. For this purpose, the iodide is usually reacted at temperatures of between −78° C. and 0°C., preferably at −60° C. to 0° C. and very especially preferably at −50° C. to −10° C. with an approximately equivalent amount, for example 1 to 1.05 equivalents, of a Grignard compound, preferably isopropylmagnesium bromide or isopropylmagnesium chloride, in an inert organic solvent, preferably an ether such as diethyl ether, tetrahydrofuran, dioxane, dimethoxyethane, methyl-tert-butyl ether or mixtures of these. The Grignard compound is subsequently scavenged at these temperatures using boric esters, preferably lower alkyl esters, very especially trimethyl borate. Work-up under acidic aqueous conditions then yields boronic acid or its trimer; or else, work-up under neutral conditions gives the esters of boronic acid (R′≠H).

Some of the iodides required for the preparation of the boronic acids III are known from the literature (for example 2-fluoro-4-chloro-5-carboisopropoxy-1-iodobenzene, CAS-No.: 264927-52-8), 2-fluoro-4-chloro-5-methoxy-1-iodobenzene (CAS-No.: 174913-22-5), 2-fluor-4-chloro-1-iodobenzene (CAS-No.: 6797-79-1) or can be prepared analogously to these methods (see also Houben-Weyl Vol. 5/4, p. 639 et seq.).

Some boronic acids according to the invention which can be prepared via this route are mentioned by way of example in Table 5:

TABLE 5

| | |
|---|---|
| | (IIIa) |
| 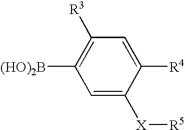 | |

| Compound | R³ | R⁴ | X—R⁵ |
|---|---|---|---|
| IIIa.1 | H | Cl | CO₂CH₃ |
| IIIa.2 | F | Cl | CO₂CH₃ |
| IIIa.3 | Cl | Cl | CO₂CH₃ |
| IIIa.4 | H | Cl | CO₂CH₂CH₃ |
| IIIa.5 | F | Cl | CO₂CH₂CH₃ |
| IIIa.6 | Cl | Cl | CO₂CH₂CH₃ |
| IIIa.7 | H | Cl | CO₂CH(CH₃)₂ |
| IIIa.8 | F | Cl | CO₂CH(CH₃)₂ |
| IIIa.9 | Cl | Cl | CO₂CH(CH₃)₂ |
| IIIa.10 | H | CN | OCH₃ |
| IIIa.11 | F | CN | OCH₃ |
| IIIa.12 | Cl | CN | OCH₃ |
| IIIa.13 | H | CN | F |
| IIIa.14 | F | CN | F |
| IIIa.15 | Cl | CN | F |

Moreover, the compounds of the formula I according to the invention can be prepared by derivatizing other 2-aryl-5-trifluoromethylpyridines.

I For example, compounds IA, where X—R⁵ is a group O—Y—R⁷ can be obtained form the respective methoxy compound IA (X—R⁵═OCH₃) by first cleaving the methyl ether and then alkylating the resulting phenol compound IA (X—R⁵═OH) with a suitable alkylating agent L-Y—R⁷, in which L is a nucleophilically displaceable leaving group, for example a halogen atom, an arylsulfonate group, a sulfate group or similar, preferably in the presence of a base.

Suitable for cleaving the methyl ethers are strong Lewis acids such as boron tribromide and also hydrohalic acids such as HBr or HI.

Preferably, the methoxy compound is reacted with 1 to 5 equivalents of the Lewis acid in an aprotic organic solvent, preferably a chlorohydrocarbon such as dichloromethane, chloroform or 1,2-dichloroethane. The reaction temperature is usually above the melting point and can be as high as the boiling point of the solvent. It is preferably in the range of from 0° C. to 50° C. Further methods and conditions for ether cleavage are described in Kocienski, “Protecting Groups”, Thieme Verlag Stuttgart 1994. The alkylation of the phenol compound IA (X—R⁵═OH) is carried out analogously to methods known from the literature (see, for example, Organikum, VEB Berlin 1988, Chapter D2, Org. Synth, Coll. Vol. III 1955, 140 and Org. Reactions. 2, 1944, 26).

II The compounds IA where X—R⁵ is NO₂, NHOH or NH₂ can be prepared from the compounds of the formula IA where R⁶═X—R⁵═H by nitration and subsequent reduction. If appropriate, an amino group R¹ will previously be protected in the known fashion.

Suitable nitrating reagents are, for example, nitric acid in various concentrations, also concentrated and fuming nitric acid, mixtures of sulfuric acid and nitric acid, also salts of nitric acid, e.g. potassium nitrate, in a mixture with sulfuric acid, also acetyl nitrates and alkyl nitrates.

The reaction can either be carried out without a solvent in an excess of the nitrating reagent or in an inert solvent or diluent, suitable substances being, for example, water, mineral acids, organic acids, halohydrocarbons such as methylene chloride, anhydrides such as acetic anhydride, and mixtures of these.

The starting compound IA {R⁶═XR⁵═H} and nitrating reagent are expediently employed in approximately equimolar amounts; as regards the conversion of the starting compound, it may be advantageous to use the nitrating reagent in an excess up to approximately 10 times the molar amount based on IA. When carrying out the reaction without solvent in the nitrating reagent, the latter is present in an even larger excess.

The reaction temperature is normally −100° C. to 200° C., preferably −30° C. to 50° C.

The compounds IA where R⁶═H and XR⁵═NO₂ can then be reduced to give compounds IA where X—R⁵═NH₂ or —NHOH:

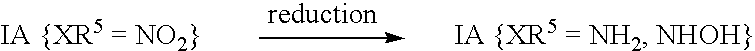

As a rule, the reduction will be carried out by reacting the nitro compound with a metal such as iron, zinc or tin under acidic reaction conditions or else with a complex hydride such as lithium aluminum hydride and sodium borohydride, the reduction being carried out in the solid state or in a solvent or diluent. Depending on the reducing agent used, suitable diluents are, for example, water, alcohols such as methanol, ethanol and isopropanol or ethers such as diethyl ether, methyl tert-butyl ether, dioxane, tetrahydrofuran and ethylene glycol dimethyl ether.

When carrying out the reduction with a metal, the process is preferably carried out in the absence of a solvent in an inorganic acid, in particular in concentrated or dilute hydrochloric acid, or in a liquid organic acid such as acetic acid or propionic acid, However, the acid can also be diluted with an inert solvent, for example one of those mentioned above. The reduction with complex hydrides is preferably carried out in a solvent, for example an ether or an alcohol.

The nitro compound IA {X—R⁵═NO₂} and the reducing agent are frequently employed in approximately equimolar amounts; to optimize the course of the reaction, it may be advantageous to use one of the two components in an excess of up to approximately 10 times the molar amount.

The amount of acid is not criticial. In order to reduce the starting compound as completely as possible, it is expedient to employ at least an equivalent amount of acid. Frequently, the acid is employed in excess, based on the nitro compound IA {X—R⁵═NO₂}.

The reaction temperature is generally in the range of from −30° C. to 200° C., preferably in the range of from 0° C. to 80° C.

For work-up, the reaction mixture is, as a rule, diluted with water and the product is isolated by filtration, crystallization or extraction with a solvent which is largely immiscible with water, for example with ethyl acetate, diethyl ether or methylene chloride. If desired, the product can subsequently be purified as usual.

The nitro group of the compounds IA {X—R⁵═NO₂} can also be hydrogenated catalytically using hydrogen. Catalysts which are suitable for this purpose are, for example, Raney nickel, palladium on charcoal, palladium oxide, platinum and platinum oxide, an amount of from 0.05 to 10.0 mol % of catalyst, based on the compound to be reduced, generally being sufficient. The process is either carried out in the absence of a solvent or in an inert solvent or diluent, for example in acetic acid, a mixture of acetic acid and water, ethyl acetate, ethanol or in toluene. After the catalyst has been removed, the reaction solution can be worked up as customary to give the product. The hydrogenation can be effected under normal hydrogen pressure or under elevated hydrogen pressure.

The resulting amino compounds, in turn, can be reacted with known electrophiles, for example with alkylsulfonyl halides or with the corresponding anhydrides to give the sulfonamides, or with alkyl halides to give the secondary or tertiary anilines.

Compounds IA in which R³ is hydrogen and X—R⁵ is NHOH can be converted into the corresponding 2-(2′-fluoro-5′-aminophenyl)pyridines (R³═F, X—R⁵═NH₂) by means of Bamberger rearrangement with HF as fluorine source. This reaction can be carried out analogously to the method described in WO 97/34872 (see following scheme).

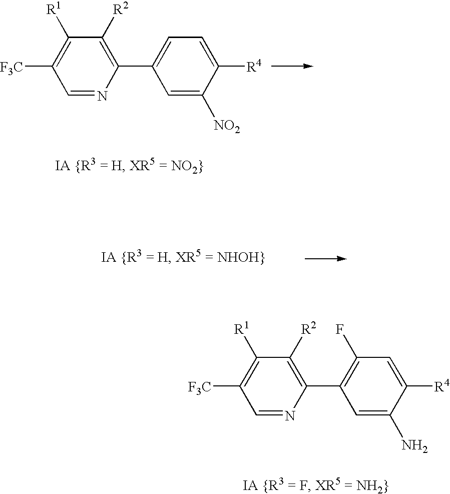

To this end, a nitro compound IA {R³═H, XR⁵═NO₂} is first hydrogenated on a platinum catalyst or a sulfur- or selenium-doped palladium catalyst in the presence of a morpholine compound, and the resulting hydroxylamine IA {R³═H, XR⁵═NHOH} is then reacted with hydrogen fluoride, yielding the fluoroamino compound {R³═F, XR⁵═NH₂}. Owing to further details on the reaction conditions, reference is made herewith to the contents of WO 97/34872.

III Further compounds I can be prepared from the 2-(5′-aminophenyl)pyridines I (X—R⁵═NH₂) by means of their diazonium salts:

X—R⁵=cyano or halogen {for example by Sandmeyer reaction: cf., for example, Houben-Weyl, Methoden der Organischen Chemie [Methods in organic chemistry], Georg Thieme Verlag Stuttgart, Vol. 5/4, 4th Edition 1960, p. 438 et seq.},

X—R⁵=hydroxyl {for example by boiling down with phenol: cf., for example, Org. Synth. Coll. Vol. 3 (1955), p. 130},

X—R⁵=mercapto or C₁-C₆-alkylthio {cf., in this context, for example Houben-Weyl, Methoden der Organischen Chemie, Georg Thieme Verlag Stuttgart, Vol. E11 1984, pp. 43 and 176},

X—R⁵=halosulfonyl {cf. in this context, for example, Houben-Weyl, Methoden der Organischen Chemie, Georg Thieme Verlag Stuttgart, Vol. E11 1984, p. 1069 et seq.},

X—R⁵=for example —CH₂—CH(halogen)-CO—O—Y—R⁸, —CH═C(halogen)-CO—O—Y—R⁷, —CH₂—CH(halogen)-PO—(O—Y—R⁷)₂, —CH═C(halogen)-CO—(O—Y—R⁷)₂ {these are generally products of a Meerwein arylation; cf. in this context, for example, C. S. Rondestredt, Org. React. 11, 189 (1960) and H. P. Doyle et al., J. Org. Chem. 42, 2431 (1977)}.

The respective diazonium salt of IA {X—R⁵═N₂⁺} is prepared, as a rule, in a manner known per se by reacting IA {X—R⁷═NH₂} with a nitrite such as sodium nitrite or potassium nitrite in an aqueous acid solution, for example in hydrochloric acid, hydrobromic acid or sulfuric acid.

To prepare the diazonium salt IA {X—R⁵═N₂⁺}, the amino compound IA {X—R⁵═NH₂} can be reacted with a nitrous ester such as tert-butyl nitrite and isopentyl nitrite under anhydrous conditions, for example in hydrogen chloride-containing glacial acetic acid, in absolute alcohol, in dioxane or tetrahydrofuran, in acetonitrile or in acetone.

The conversion of the resulting diazonium salt into the corresponding compound IA where X—R⁵=cyano, chlorine, bromine or iodine is especially preferably carried out by treatment with a solution or suspension of a copper(I) salt such as copper(I) cyanide, copper(I) chloride, copper(I) bromide and copper(I) iodide, or with an alkali metal salt solution.

The conversion of the resulting diazonium salt into the corresponding hydroxy compound IA {X—R⁵=hydroxyl} is expediently carried out by treating the diazonium salt IA with an aqueous acid, preferably sulfuric acid. The addition of a copper(II) salt such as copper(II) sulfate can have an advantageous effect on the course of the reaction. In general, this reaction is carried out at from 0° C. to 100° C., preferably at the boiling point of the reaction mixture.

Compounds IA where X—R⁵=mercapto, C₁-C₆-alkylthio or halosulfonyl are obtained, for example, by reacting the corresponding diazonium salt of IA with hydrogen sulfide, an alkali metal sulfide, a dialkyl disulfide such as dimethyl disulfide, or with sulfur dioxide.

The Meerwein arylation is usually the reaction of the diazonium salts with alkenes or alkynes. The alkene or alkyne is preferably employed in an excess up to approximately 3000 mol % based on the amount of the diazonium salt. Thus, for example, the reaction of the diazonium salt IA {X—R⁵═N₂⁺} with acrylic esters of the formula H₂C═CH—COO—Y—R⁷, preferably in the presence of copper salts such as Cu(I) halide or Cu(II) halide, for example Cu(I)Cl or Cu(II)Cl₂, yields compounds I where X—R⁵═H₂C—CH(Hal)-COO—Y—R⁷.

The above-described reactions of the diazonium salt IA {X—R⁵═N₂⁺} can be carried out, for example, in water, in aqueous hydrochloric acid or hydrobromic acid, in a ketone such as acetone, diethyl ketone and methyl ethyl ketone, in a nitrile such as acetonitrile, in an ether such as dioxane and tetrahydrofuran, or in an alcohol such as methanol and ethanol.

Unless otherwise stated for the individual reactions, the reaction temperatures are normally from −30° C. to 50° C.

All reactants are preferably employed in approximately stoichiometric amounts, with an excess of one or the other component of up to approximately 3000 mol % also being advantageous.

The mercapto compounds IA {X—R⁵═SH} can also be obtained by reducing the compounds IA where X—R⁷=halosulfonyl which are described hereinbelow. Examples of reducing agents which can be used are transition metals such as iron, zinc and tin (cf., in this context, for example “The Chemistry of the Thiol Group”, John Wiley, 1974, p. 216).

IV Halosulfonation of 4-aryl-1-difluoromethoxyimidazoles IA, where XR⁵ is hydrogen:

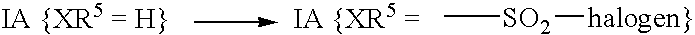

The halosulfonation can be carried out without solvent in an excess of sulfonating reagent or in an inert solvent/diluent, for example in a halogenated hydrocarbon, an ether, an alkyl nitrile or a mineral acid.

Chlorosulfonic acid constitutes both the preferred reagent and a suitable solvent.

The sulfonating reagent is normally employed in slightly substoichiometric amounts (of up to approximately 95 mol %) or in an excess of 1 to 5 times the molar amount based on the starting compound IA (where X—R⁵═H). If the process is carried out without inert solvent, an even larger excess may also be expedient.

The reaction temperature is normally between 0° C. and the boiling point of the reaction mixture.

For work-up, the reaction mixture is treated with, for example, water, whereupon the product can be isolated as usual. The halosulfonated compounds IA {X—R⁵═SO₂C₁}, in turn, are valuable starting materials for compounds IA where X—R⁵═SH, S—Y—R⁷, SO₂OYR⁷ and SO₂—N(Y—R⁷)(Z-R⁸).

The compounds I where X—R⁵═CO—Y—R⁷ are advantageously prepared from 2-(5′-alkoxycarbonylphenyl)pyridines I {X—R⁵ CO₂Rx where R^{x}═C₁-C₄-alkyl}. The latter can be obtained in a particularly efficient manner by the above-described coupling of pyridines II with boronic acids IIIa.

To this end, the following choice of procedures exists:

Hydrolyzing the ester group CO₂Rx to give the free acid, converting the acid into its mixed anhydride with formic acid or carbonic acid and reducing the anhydride with borohydrides such as NaBH₄ or reducing the free acid directly with borane adducts such as the BH₃/dimethyl sulfide complex or the BH₃/THF complex to give the alcohol IA {X—R⁵═CH₂OH} and oxidizing the alcohol I to give the aldehyde IA {X—R⁵═CHO}.

Preparation of the acid chloride IA {X—R⁵═COCl} via free acid and reduction with complex hydrides at low temperature to give the aldehyde directly.

The skilled worker is sufficiently familiar with the methods required for this purpose, for example Larock “Comprehensive Organic Transformations” VCH 1989 Weinheim or Fuhrhop, Penzlin, “Organic Sythesis” VCH Verlag Weinheim 1986.

The 2-(3′-formylphenyl)pyridines IA {X—R⁵═CHO} obtained in this manner can then be reacted further analogously to the processes described in EP-A 240569 and DE-A 3904082, for example in a Wittig reaction. Thus, for example pyridylcinnamic acids/pyridylcinnamic esters IA {X—R⁵═CH═CH—COO—Y—R⁷ or CH═C(Rz)-COO—Y—R⁷ where R^{z}=halogen or C₁₋₄-alkyl} can be prepared. The phosphonium salts, phosphonates or phosphoros ylides required as reactants for this purpose are known or can be synthesized in a manner known per se {cf., in this context, for example Houben-Weyl, Methoden der Organischen Chemie, Vol. El, pp. 636 et seq. and Vol. E2, pp. 345 et seq., Georg Thieme Verlag Stuttgart 1982; Chem. Ber. 95, 1962, 3993}.

The 2-(3′-formylphenyl)pyridines IA can also be converted into compounds IA where X—R⁵═—CO—Y—R⁷ in a manner known per se, for example by reacting them with a suitable organometallic compound Me—Y—R⁷ where Me is a base metal, preferably lithium or magnesium, and subsequently oxidizing the resulting alcohols (cf., for example, J. March, Advanced

Organic Chemistry, 3rd ed., John Wiley, New York 1985, pp. 816 et seq. and 1057 et seq.).

The compounds IA where X—R⁵═—CO—Y—R⁷, in turn, can be reacted further in a Wittig reaction in the manner described above for the aldehydes.

Further possibilities of preparing other 2-aryl-5-trifluoromethylpyridines IA from compounds IA where X—R⁵=formyl include aldol condensation, which is known per se, and Knoevenagel or Perkin condensation reactions. Suitable conditions for these processes can be found, for example, in Nielson, Org. React. 16, 1968, 1 et seq. {aldol condensation}; Org. React. 15, 1967, 204 et seq. {Knoevenagel condensation} and Johnson, Org. React. 1, 1942, 210 et seq. {Perkin condensation}.

The compounds IA where X—R⁵═—CO—Y—R⁷ can also be converted into their corresponding oximes X—R⁵═C(YR⁷)(═NOR⁹) in a manner known per se {cf. in this context, for example, Houben-Weyl, Methoden der Organischen Chemie, Georg Thieme Verlag Stuttgart, Vol. 10/4, 4th Edition 1968, p. 55 et seq. and p. 73 et seq.}.

VI The compounds of the formula IB where XR⁵ and R⁴ form a chain of the formula —O—(CR¹⁵, R¹⁶)ₖCON(R¹⁷)— or —S—(CR¹⁵, R¹⁶)ₖCON(R¹⁷)— can be prepared by coupling, as described above, a halopyridine II with a corresponding boronic acid III (compound III where Met=B(OR′)₂, where R⁴, together with X—R⁵, is O—C(R¹⁵, R¹⁶)ₖ—CO—N(R¹⁷)— or —S—(CR¹⁵, R¹⁶)ₖCON(R¹⁷)—). A further preparation method starts from the aminophenols IA {R⁴═OH and X—R⁵═NH₂ or R⁴═NH₂ and X—R⁵═OH} or aminothiophenols IA {R⁴═SH and X—R⁵═NH₂ or R⁴═NH₂ and X—R⁵═SH}, which are cyclized by known methods (see, for example, U.S. Pat. No. 4,798,620, WO 95/02590, WO 98/07720) using α-halocarboxylic acids or their esters or derivatives of similar reactivity to give the compounds IB (for example in analogy with synthesis scheme 6 of WO 98/07720). The amino(thio)phenols IA required can be prepared by the methods described under II.

Table 6 shows examples of preferred aminophenols of the formula IAr where R¹, R² and R³ have the abovementioned meanings, in particular meanings stated in Table 6:

TABLE 6

| | |
|---|---|
| | (IAr) |
| 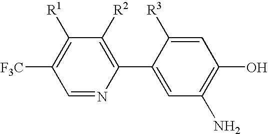 | |

| | R¹ | R² | R³ |
|---|---|---|---|
| IAr.1 | CH₃ | F | F |
| IAr.2 | CH₃ | F | Cl |
| IAr.3 | CH₃ | Cl | F |
| IAr.4 | CH₃ | Cl | Cl |

The compounds IB which can be obtained in this manner, in which R¹⁷ is hydrogen, can be reacted with an alkylating agent R^{17′}-L by methods known per se as are described, for example, in WO 95/02590, WO 98/07700 and the prior art described therein, in Sicker et al. Tetrahedron 52, 1996, 10389 or in DE-A 19508590. L is a nucleophilically displaceable leaving group such as halogen, arylsulfonate, triflate or sulfate, or an isocyanate group. R^{17′} is, for example, C₁-C₆-alkyl, C₁-C₆-haloalkyl, C₂-C₆-alkenyl, C₂-C₆-haloalkenyl, C₂-C₆-alkynyl, C₁-C₄-alkylsulfonyl, C₁-C₄-haloalkylsulfonyl, C₁-C₄-alkylcarbonyl, C₁-C₄-haloalkylcarbonyl, C₁-C₄-alkoxycarbonyl, C₁-C₄-alkoxy-C₁-C₄-alkyl, C₁-C₄-alkoxycarbonyl-C₁-C₄-alkyl, mono- and di(C₁-C₄-alkyl)aminocarbonyl-C₁-C₄-alkyl, phenyl-C₁-C₄-alkyl, C₃-C₈-cycloalkyl, C₃-C₈-cycloalkyl-C₁-C₄-alkyl, 3-, 4-, 5-, 6- or 7-membered heterocyclyl-C₁-C₄-alkyl. If L is a nucleophilically diplaceable leaving group, the reaction with the alkylating agent R^{17′}-L is, as a rule, carried out in the presence of a base.

The compounds I in which the radicals X—R⁵ and R⁴ form a chain of the formula —O—C(R¹⁵, R¹⁶)—CO—NR¹⁷— or —S—C(R¹⁵, R¹⁶)—CO—NR¹⁷-can also be obtained by reductive cyclization of nitrophenoxycarboxylic acid derivatives of the formula IAs or of corresponding nitrothiophenoxycarboxylic acid derivatives. In the case of the nitrophenoxycarboxylic acid derivatives IAs, compounds IB are first formed in which X—R⁵ and R⁴ form a chain of the formula —O—C(R¹⁵, R¹⁶)—CO—N(O)₁H— where l=0 or 1. These can subsequently be functionalized. An example of such a synthesis sequences is shown in Scheme 3:

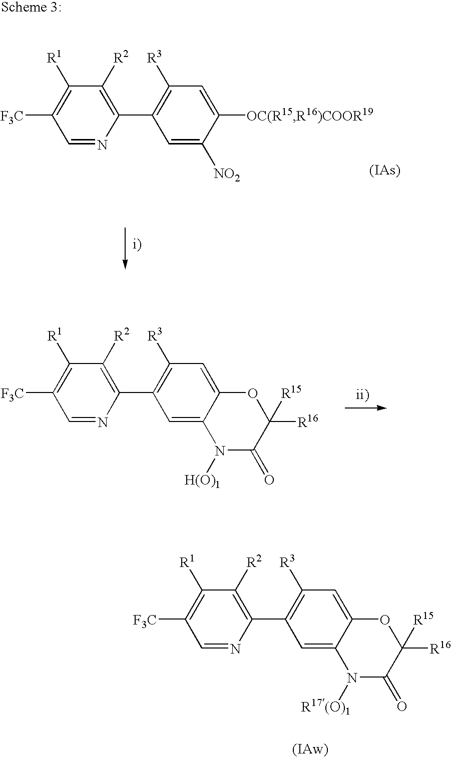

In the formula IAw in Scheme 3, R¹, R², R³, R¹⁵, R¹⁶ and R^{17′} have the abovementioned meanings. R¹⁹ is alkyl having, preferably, 1 to 4 C atoms, in particular methyl or ethyl. The variable l is 0 or 1. In Scheme 3, step i) is the reductive cyclization and step ii) is the above-described reaction with the electrophile L-R^{17′}.

The nitro(thio)phenoxyalkanecarboxylic acid derivatives IAs can be prepared and cyclized reductively to give the compounds IB for example in analogy to the prior art stated in Böger, “Peroxidizing Herbicides”, Springer Verlag, Berlin 1999, p. 32, or in analogy with the methods described by Sicker et al., Synthesis, 1989, p. 211; Atkinson et al. J. Org. Chem. 56, (1991) p. 1788; Coutts et al. J. Chem. Soc., 1963, S. 4610, U.S. Pat. No. 3,862,180, WO 95/02590 and the literature cited therein, DE-A 19508590, Sicker et. al. J. Het. Chem. 31, 1994, p.801, WO 98/07720 and international application PCT/EP 00/08639. Table 7 shows examples of preferred nitrophenoxyalkanecarboxylic acid derivatives of the formula IAs where R¹⁵ and R¹⁶ are hydrogen and R¹, R², R³ and R¹⁹ have the abovementioned meanings, in particular the meanings mentioned in Table 7, and which are of particular importance as intermediates for the preparation of compound B:

TABLE 7

| | |
|---|---|
| | (IAs) |
| 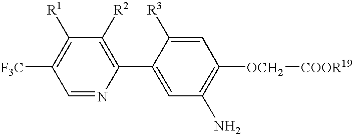 | |

| | R¹ | R² | R³ | R¹⁹ |
|---|---|---|---|---|
| IAs.1 | CH₃ | F | F | CH₃ |
| IAs.2 | CH₃ | F | F | CH₂CH₃ |
| IAs.3 | CH₃ | F | Cl | CH₃ |
| IAs.4 | CH₃ | F | Cl | CH₂CH₃ |
| IAs.5 | CH₃ | Cl | F | CH₃ |
| IAs.6 | CH₃ | Cl | F | CH₂CH₃ |
| IAs.7 | CH₃ | Cl | Cl | CH₃ |
| IAs.8 | CH₃ | Cl | Cl | CH₂CH₃ |
| IAs.9 | NH₂ | F | F | CH₃ |
| IAs.10 | NH₂ | F | F | CH₂CH₃ |
| IAs.11 | NH₂ | F | Cl | CH₃ |
| IAs.12 | NH₂ | F | Cl | CH₂CH₃ |
| IAs.13 | NH₂ | Cl | F | CH₃ |
| IAs.14 | NH₂ | Cl | F | CH₂CH₃ |
| IAs.15 | NH₂ | Cl | Cl | CH₃ |
| IAs.16 | NH₂ | Cl | Cl | CH₂CH₃ |

VII 4- or 8-(5′-Trifluoromethylpyridyl)benzazoles of the formula IC (compounds IC where X—R⁵ and R⁶ are a chain —N═C(R¹⁸)—O— or —N═C(R¹⁸)—S—) can be obtained in various ways, in particular by one of the following processes (see also WO 98/27090 and WO 99/55702, whose technical teaching can be applied to the preparation of the compounds IC):

A Reaction of a (3-aminophenyl)-5-trifluormethylpyridine of the formula IA {X—R⁵═NH₂) with halogen and ammonium thiocyanate or with an alkali metal thiocyanate or alkaline earth metal thiocyanate in accordance with the following scheme:

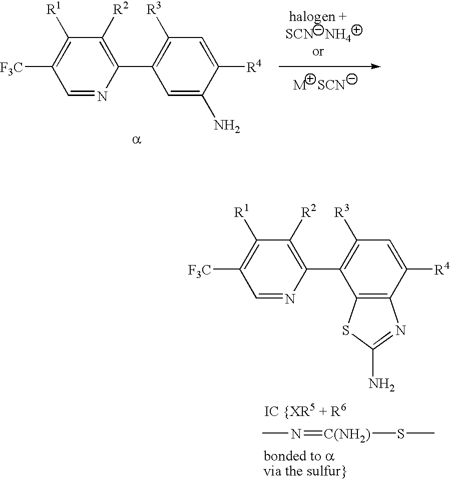

M⊕=alkali metal ion or ½ alkaline earth metal ion

Preferred halogen is chlorine or bromine; among the alkali metal thiocyanates and alkaline earth metal thiocyanates, sodium thiocyanate is preferred.

As a rule, the process is carried out in an inert solvent/diluent, for example in a hydrocarbon such as toluene and hexane, in a halogenated hydrocarbon such as dichloromethane, in an ether such as tetrahydrofuran, in an alcohol such as ethanol, in a carboxylic acid such as acetic acid, or in an aprotic solvent such as dimethylformamide, acetonitrile and dimethyl sulfoxide.

The reaction temperature is usually above the melting point and can be up to the boiling point of the solvent. It is preferably in the range of from 0 to 150° C.

To achieve as high as possible a yield of product of interest, halogen and ammonium thiocyanate, or alkali metal thiocyanate/alkaline earth metal thiocyanate, are employed in equimolar amounts or in an excess of up to approximately 5 times the molar amount based on the amount of (3-aminophenyl)-5-trifluoromethylpyridine IA {X—R⁵═NH₂}.

A variant of the process consists in first reacting the (3-aminophenyl)-5-trifluoromethylpyridine IA {X—R⁵═NH₂} with ammonium thiocyanate or an alkali metal thiocyanate or alkaline earth metal thiocyanate to give a thiourea IA {X—R⁵═NH—C(S)—NH₂}

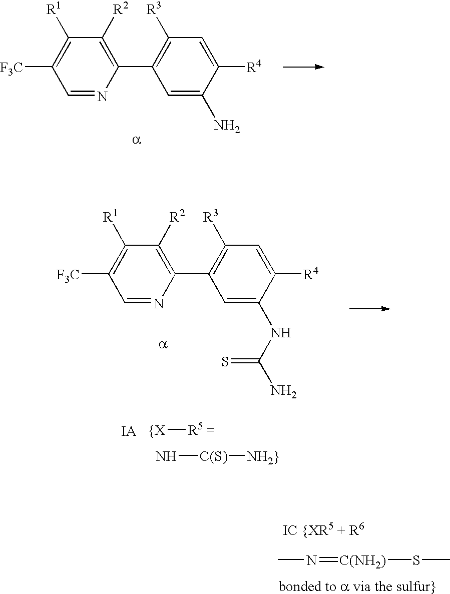

and subsequently to convert the thiourea IA {X—R⁵═NH—C(S)—NH₂} into 8-(5′-trifluoromethylpyridyl)benzothiazole IA {X—R⁵+R⁴═—N═C(NH₂)—S—} by treatment with halogen. The amino group in the 2-position on the thiazole radical can be functionalized in a known manner, for example via its diazonium compound (R¹⁸═N₂⁺).

The compounds of the formula IC in which R⁶ and X—R⁵ are a chain —S—C(R¹⁸)═N— with the nitrogen being bonded via the α carbon atom can be prepared analogously.

B Diazotization of a (3-aminophenyl)-5-trifluormethylpyridine IA {R⁶═H, X—R⁵═NH₂}, conversion of the respective diazonium salt into a (3-azidophenyl)-5-trifluoromethylpyridine IA {R⁶═H, X—R⁵═N₃} and its reaction with a carboxylic acid R¹⁸COOH or a derivative thereof in accordance with scheme 4 hereinbelow, giving rise to a compound IC in which R⁶ and X—R⁵ are a chain —O—C(R¹⁸)═N— with the oxygen being bonded via the a carbon atom.

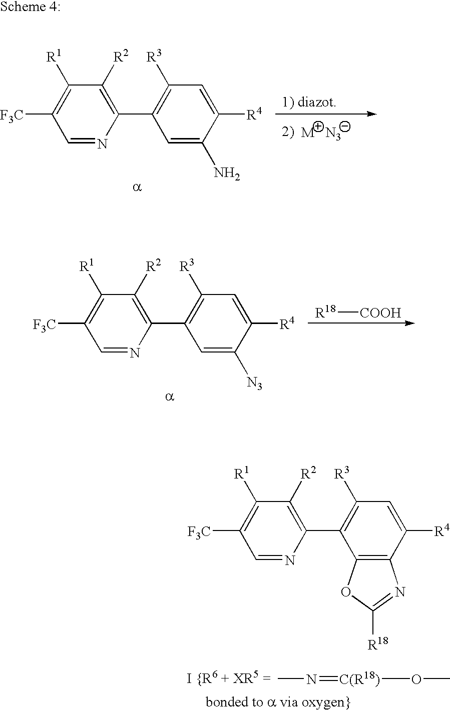

M⊕ is an alkali metal ion or ½ alkaline earth metal ion.

What has been said above also applies to the diazotization process. The conversion into the aryl azide IA {R⁶═H, X—R⁵═N₃} is preferably carried out by reacting the diazonium compounds {R⁶═H, X—R⁵═N₂+} with an alkali metal azide or alkaline earth metal azide such as sodium azide or by reaction with trimethylsilyl azide.

The reaction of the azides IA {R⁶═H, X—R⁵═N₃} with a carboxylic acid as shown in Scheme 4 is either carried out in an inert solvent, for example an ether such as tetrahydrofuran and dioxane, an aprotic solvent such as dimethylformamide and acetonitrile, a hydrocarbon such as toluene and hexane, a halogenated hydrocarbon such as dichloromethane, or without a solvent in an excess of the carboxylic acid R¹⁸—COOH. In the latter case, the addition of a mineral acid such as phosphoric acid may be helpful.

The reaction is preferably carried out at elevated temperature, for example at the boiling point of the reaction mixture.

VIII If desired, the 2-aryl-5-trifluoromethylpyridines of the formula I where m=0 can be converted by oxidation on the nitrogen to give the pyridine-N-oxides of the formula I where m=1, which also have a herbicidal and desiccant/defoliant action.

The oxidation of the pyridines to give the N-oxides can be carried out in analogy to known methods, for example by the methods described by A. Albini, S. Pietra in “Heterocyclic N-Oxides” CRC-Press Inc, Boca Raton USA 1991; Mosher et al. Org. Synth. Coll Vol. IV, 1963 page 828; Taylor et al., Org. Synth. Coll Vol. IV, 1963 page 704; Bell et al., Org. Synth. 69, 226, 1990; and JP 20000191644.

Oxidants which are customary for converting the pyridines I into their N-oxides are, for example, peracetic acid, trifluoroperacetic acid, perbenzoic acid, meta-chloroperbenzoic acid, magnesium monoperphthalate, 1,2-dicarboxylic acid derivatives in general, sodium perborate, oxone (contains peroxodisulfate), pertungstic acid, hydrogen peroxide, methyltrioxorhenium. These reagents can be used alone or as a mixture.

The oxidation is preferably carried out in a solvent or diluent. Suitable solvents are water, sulfuric acid, carboxylic acids such as, for example, acetic acid, and halogenated solvents such as, for example, dichloromethane and chloroform, or else mixtures of the above.

The reaction is normally carried out in a temperature range of from 0° C. to the boiling point of the solvent, preferably up to 150° C.

The oxidants are normally employed in at least equimolar amounts, frequently in a large excess of, for example, up to 5 equivalents based on the pyridine I to be oxidized.

In the case of the 4-aminopyridines I {R¹═NH₂} it may be necessary to protect the amino nitrogen and then to eliminate the protecting group when the reaction has ended, depending on the oxidant. Protecting groups which are suitable for this purpose and the conditions suitable for their introduction and elimination are found in Kocienski, “Protecting Groups”, Thieme Verlag Stuttgart 1994. Examples of suitable protecting groups which may be mentioned are benzyloxycarbonyl and fluorenylmethoxycarbonyl.

The examples which follow are intended to illustrate the invention in greater detail without imposing any limitation.

### PREPARATION EXAMPLES

### Example 1

### Preparation of 2-(2-fluoro-4-chloro-5-methoxyphenyl-1-yl)-3-chloro-4-methyl-5-trifluoromethylpyridine (IAa.3)

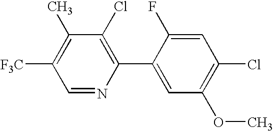

1.1 2-Benzyloxy-3-chloro-5-trifluoromethylpyridine

21.6 g (0.20 mol) of benzyl alcohol were added to a solution of 43.2 g (0.20 mol) of 2,3-dichloro-5-trifluoromethylpyridine in 250 ml of DMF, followed by the portionwise additon of 22.4 g (0.20 mol) of potassium tert-butoxide. The reaction mixture was stirred overnight, introduced into 11 of saturated ammonium chloride solution and then extracted three times using in each case 300 ml of methyl tert-butyl ether. After the combined organic phases had been washed with water and dried over sodium sulfate, the solvent was removed in vacuo. This gave 54.1 g of 2-benzyloxy-3-chloro-5-trifluoromethylpyridine, which was reacted in the next step without further purification.

¹H NMR (CDCl₃): δ (ppm)=8.3 (s, 1H), 7.8 (m, 1H), 7.5 to 7.3 (m, 5H), 5.5 (s, 2H).

1.2 2-Benzyloxy-3-chloro-4-methyl-5-trifluoromethylpyridine

57 ml of a butyllithium solution (1.3 M in hexane) were added dropwise at −70° C. to a solution of 20.0 g (0.07 mol) of 2-benzyloxy-3-chloro-5-trifluoromethylpyridine of Example 1.1 in 100 ml of THF, and stirring was continued for 30 minutes at −70° C. This solution was subsequently added dropwise at −70° C. to a solution of 29.6 g (0.21 mol) of methyl iodide in 100 ml of THF, and stirring was continued for 90 minutes at −70° C.

After heating to −10° C., 200 ml of a saturated ammonium chloride solution were added, the mixture was diluted with 200 ml of a saturated sodium chloride solution and extracted three times using in each case 200 ml of methyl tert-butyl ether. After drying of the combined organic phases, the mixture was concentrated, yielding 20.4 g of 2-benzyloxy-3-chloro-4-methyl-5-trifluoromethylpyridine of 90% purity.

¹H NMR (CDCl₃): δ (ppm)=8.3 (s, 1H), 7.5 to 7.3 (m, 5H), 5.5 (s, 2H), 2.5 (s, 3H).

1.3 2-Hydroxy-3-chloro-4-methyl-5-trifluoromethylpyridine

15.5 g (0.14 mol) of trimethylsilyl chloride were added to 21.4 g (0.14 mol) of sodium iodide in 250 ml of acetonitrile, the mixture was stirred for 15 minutes, and a solution of 28.7 g (0.095 mol) of 2-benzyloxy-3-chloro-4-methyl-5-trifluoromethylpyridine of Example 1.2 in 50 ml of acetonitrile was subsequently added dropwise at room temperature. The mixture was then stirred for 1 hour at 50° C. After removal of the solvent in vacuo, ice-cold water was carefully added to the residue, and the mixture was extracted three times using in each case 200 ml of methyl tert-butyl ether. The combined organic phases were dried over sodium sulfate and the solvent was removed in vacuo. Chromatography of the residue on silica gel with cyclohexane/ethyl acetate (4/1, v/v) yielded 17.4 g of 2-hydroxy-3-chloro-4-methyl-5-trifluoromethylpyridine of m.p. 204 to 205° C.

¹H NMR (CDCl₃): δ (ppm)=8.8 (s, 1H), 2/5 (s, 3H).

1.4 2,3-Dichloro-4-methyl-5-trifluoromethylpyridine

14.4 g (0.068 mol) of 2-hydroxy-3-chloro-4-methyl-5-trifluoromethylpyridine of Example 1.3 in 100 ml of phosphorus oxychloride were heated for 3 hours at 75° C. The reaction mixture was subsequently added dropwise to 1.51 of water/300 ml of methylene chloride with stirring, the organic phase was then separated off, and the aqueous phase was extracted twice more with in each case 300 ml of methylene chloride. After the combined organic phases had been dried, these were concentrated in vacuo, and the crude product was distilled in vacuo (b.p. 78 to 80° C. at 16 mm). This gave 9.4 g of 2,3-dichloro-4-methyl-5-trifluoromethylpyridine.

¹H NMR (CDCl₃): δ (ppm)=8.5 (s, 1H), 2.6 (s, 3H).

1.5 2-(2-Fluoro-4-chloro-5-methoxyphenyl-1-yl)-3-chloro-4-methyl-5-trifluoromethylpyridine

4.5 g (0.019 mol) of the dichloropyridine of Example 1.4, 4.0 g (0.019 mol) of 2-fluoro-4-chloro-5-methoxyphenylboronic acid, 1.1 g (0.001 mol) of tetrakistriphenylphosphine palladium and 12.0 g of sodium hydrogencarbonate in 150 ml of THF and 150 ml of water were refluxed for 20 hours with stirring. After cooling, the phases were separated, the aqueous phase was extracted twice using in each case 100 ml of methyl tert-butyl ether, and the combined organic phases were dried over sodium sulfate and concentrated in vacuo. Chromatography of the residue on silica gel with cyclohexane/ethyl acetate (100:1, v/v) yielded 2.4 g of 2-(2-fluoro-4-chloro-5-methoxyphenyl-1-yl)-3-chloro-4-methyl-5-trifluoromethylpyridine.

¹H NMR (CDCl₃): δ (ppm)=8.8 (s, 1H), 7.3 (d, 1H), 7.0 (d, 1H), 3.9 (s, 3H), 2.6 (s, 3H).

### Example 2

### 2-(2-Fluoro-4-chloro-5-hydroxyphenyl)-3-chloro-4-methyl-5-trifluoromethylpyridine (compound IAa.2)

27 ml (0.027 mol) of a boron tribromide solution (1 M in methylene chloride) were added dropwise at 0° C. to a solution of 2.4 g (0.007 mol) of the pyridine of Example 1.5 in 50 ml of dichloromethane. After the reaction mixture had been stirred for two hours at room temperature, ice-cold water was added, and the phases were subsequently separated. The aqueous phase was extracted twice using in each case 100 ml of methylene chloride. The combined organic phases were dried and concentrated in vacuo. This gave 2 g of 2-(2-fluoro-4-chloro-5-hydroxyphenyl)-3-chloro-4-methyl-5-trifluoromethylpyridine.

¹H NMR (CDCl₃): δ (ppm)=8.8 (s, 1H), 7.2 (d, 1H), 7.0 (s, 1H), 2.5 (s, 3H).

### Example 3

### 2-(2-Fluoro-4-chloro-5-propargyloxyphenyl)-3-chloro-4-methyl-5-trifluoromethylpyridine (Compound IAa.10)

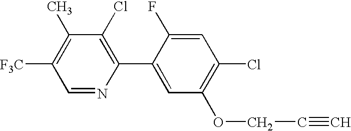

0.37 g (2.6 mmol) of potassium carbonate and 0.23 g (19.4 mmol) of propargyl bromide were added in succession to a solution of 0.6 g (1.7 mmol) of the phenol of Example 2 in 10 ml of dimethylformamide (DMF). The mixture was stirred for 4 hours at room temperature. The reaction mixture was subsequently introduced into ice-cold water and the mixture was extracted three times with methyl tert-butyl ether. The combined organic phases were washed with water and dried over sodium sulfate. Chromatography on silica gel with cyclhexane/ethyl acetate (9/1, v/v) gave 0.62 g of the title compound of m.p. 95 to 98° C.

¹H NMR (CDCl₃): δ (ppm)=8.8 (s, 1H), 7.3 (d, 1H), 7.1 (d, 1H), 4.8 (d, 2H), 2.6 (s, 3H), 2.5 (t, 1H).

### Example 4

### 2-(2-Fluoro-4-chloro-5-isopropoxycarbonylphenyl-1-yl)-3-chloro-4-methyl-5-trifluoromethylpyridine (IAa.131)

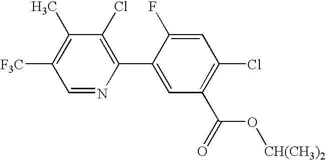

4.1 Isopropyl 2-chloro-4-fluoro-5-iodobenzoate

20.0 g (0.086 mol) of isopropyl 2-chloro-4-fluoro-5-aminobenzoate (CAS-No. 86819-51-4) were introduced into 100 ml of concentrated hydrochloric acid at 0° C., and a solution of 6.6 g (0.095 mol) of sodium nitrite in 20 ml of water was added dropwise at 0 to 5° C. Stirring was continued for 1 hour at 0° C., a solution of 2.6 g (0.043 mol) of urea in 20 ml of water was then added dropwise and the mixture was stirred for a further 15 minutes. The reaction mixture was subsequently added dropwise to a solution of 17.2 g (0.1 mol) of potassium iodide in 30 ml of water. The mixture was first allowed to come to room temperature and was subsequently warmed for 30 minutes at 60 to 70° C. After cooling, the mixture was extracted three times with in each case 200 ml of methylene chloride, and the combined organic phases were dried over sodium sulfate and concentrated. This gave 27.6 g of isopropyl 2-chloro-4-fluoro-5-iodobenzoate of m.p. 38 to 43° C.

¹H NMR (CDCl₃): δ (ppm)=8.2 (d, 1H), 7.2 (d, 1H), 5.2 (sept, 1H), 1.4 (d, 6H).

4.2 2-Fluoro-4-chloro-5-isopropoxycarbonylphenylboronic Acid

7.7 ml (0.015 mol) of an isopropylmagnesium chloride solution (2 M in ether) were added dropwise at −40° C. to a solution of 5.0 g (0.015 mol) of the iodide of Example 4.1 in 30 ml of methyl tert-butyl ether, and stirring was then continued for 1 hour at −40° C. A solution of 4.6 g (0.043 mol) of trimethyl borate in 10 ml of THF was subsequently added dropwise, stirring was continued for 1 hour at −40° C., and the mixture was allowed to come to room temperature. The mixture was treated with 50 ml of 10% strength hydrochloric acid and extracted three times with in each case 50 ml of methyl tert-butyl ether, and the combined organic phases were dried over sodium sulfate and subsequently concentrated. Recrystallization from n-hexane yielded 2.5 g of 2-fluoro-4-chloro-5-isopropoxycarbonylphenylboronic acid of m.p. 176 to 180° C., which in some cases also contained some trimeric boron oxine.

¹H NMR (d₆-DMSO): δ (ppm)=8.4 (br, 2H), 8.0 (d, 1H), 7.4 (d, 1H), 5.2 (sept, 1H), 1.4 (d, 6H).

4.3 2-(2-Fluoro-4-chloro-5-isopropoxycarbonylphenyl)-3-chloro-4-methyl-5-trifluoromethylpyridine

1.8 g (7.7 mmol) of the pyridine of Example 1.4 and 2 g (7.7 mmol) of the boronic acid of Example 4.2 were reacted analogously to the procedure described in Example 1.5, yielding 1.0 g of the title compound.

¹H NMR (CDCl₃): δ (ppm)=8.8 (s, 1H), 8.0 (d, 1H), 7.3 (d, 1H), 5.3 (sept, 1H), 2.6 (s, 3H), 1.4 (d, 6H).

### Example 5

### 2-(2-Fluoro-4-chloro-5-nitrophenyl)-3-chloro-4-methyl-5-trifluoromethylpyridine (Compound IAa.81)

1.87 g (29.6 mmol) of 100% strength nitric acid were added dropwise at 0 to 5° C. to a solution of 8.0 g (24.7 mmol) of 2-(2-fluoro-4-chlorophenyl-1-yl)-3-chloro-4-methyl-5-trifluoromethylpyridine (prepared analogously to the procedure described in Example 1.5 starting from the pyridine of Example 1.4 and 2-fluoro-4-chlorophenylboronic acid) in 100 ml of concentrated sulfuric acid, and stirring was continued for 3 hours at this temperature. The reaction mixture was subsequently introduced into 500 ml of ice-cold water and the mixture was extracted three times with in each case 200 ml of ethyl acetate. After the combined organic phases had been dried over sodium sulfate and the solvent had been removed, the residue which remained was filtered through a short silica gel column (eluent cyclohexane/ethyl acetate=4/1, (v/v)). This gave 3.8 g of 2-(2-fluoro-4-chloro-5-nitro-phenyl-1-yl)-3-chloro-4-methyl-5-trifluoromethylpyridine.

¹H NMR (CDCl₃): δ (ppm)=8.8 (s, 1H), 8.2 (d, 1H), 7.4 (d, 1H), 2.6 (s, 3H).

### Example 6

### 2-(2-Fluoro-4-chloro-5-nitrophenyl)-3-chloro-4-methyl-5-trifluoromethylpyridine (Compound IAa.83)

2.3 g of iron powder were refluxed in 600 ml of 100% strength acetic acid and a solution of 3.8 g (10.3 mmol) of the product of Example 5 in 40 ml of methanol were added dropwise. The mixture was then warmed for 2 hours at 80° C., and the methanol was subsequently removed. Approximately 500 ml of ethyl acetate were added, and the mixture was introduced into ice-cold water. The ethyl acetate phase was removed and the aqueous phase was extracted twice more with in each case 200 ml of ethyl acetate. After the combined organic phases had been dried over sodium sulfate, the solvent was removed in vacuo. This gave 3.0 g of the amino compound which was reacted further without further purification.

¹H NMR (CDCl₃): δ (ppm)=8.8 (s, 1H), 7.2 (d, 1H), 6.8 (d, 1H), 4.2 (br, 2H), 2.6 (s, 3H).

### Example 7

### 2-(2-Fluoro-4-chloro-5-(2-chloro-2-carbomethoxyethyl)-phenyl)-3-chloro-4-methyl-5-trifluoromethylpyridine (IAa.199)

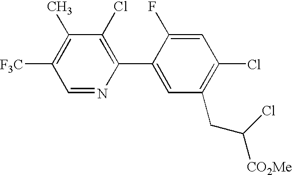

A mixture of 0.91 g (8.9 mmol) of tert-butyl nitrite, 0.51 g (5.9 mmol) of methyl acrylate and 0.99 g (7.3 mmol) of CuCl in 50 ml of acetonitrile was treated with 2.0 g (5.9 mmol) of 2-(2-fluoro-4-chloro-5-aminophenyl-1-yl)-3-chloro-4-methyl-5-trifluoromethylpyridine, and the mixture was stirred for 10 hours at 0° C. After the solvent had been removed, the residue was chromatographed on silica gel with cyclonexane/ethyl acetate (1/1, v/v), yielding 0.22 g of the title compound.

¹H NMR (CDCl₃): δ (ppm)=8.8 (s, 1H), 7.4 (d, 1H), 7.2 (d, 1H), 4.6 (m, 1H), 3.8 (s, 3H), 3.5 (m, 1H), 3.3 (m, 1H), 2.6 (s, 3H).

### Example 8

### 2-(2-Fluoro-4-chloro-5-hydroxycarbonylphenyl-1-yl)-3-chloro-4-methyl-5-trifluoromethylpyridine (IAa.124)

0.6 g of 2-(2-fluoro-4-chloro-5-isopropoxycarbonylphenyl)-3-chloro-4-methyl-5-trifluoromethylpyridine of Example 4.3 was refluxed for 3 hours in 40 ml of glacial acetic acid together with concentrated hydrochloric acid. The mixture was subsequently evaporated to dryness in vacuo. This gave the title compound in quantitative yield.

¹H NMR (CDCl₃): δ (ppm)=8.8 (S, 1H), 8.2 (d, 1H), 7.4 (d, 1H), 2.6 (s, 3H).

### Example 9

### 2-[2-Fluoro-4-chloro-5-(2-methoxycarbonylpropionyl)-carbonylphenyl-1-yl]-3-chloro-4-methyl-5-trifluoromethylpyridine (compound IAa.143 as R enantiomer and as S enantiomer)

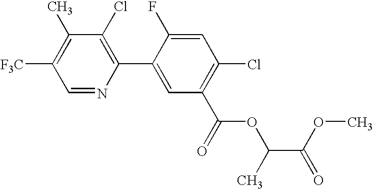

0.5 g (1.3 mmol) of the acid of Example 8 was treated with 5 ml of thionyl chloride and the mixture was subsequently refluxed for 3 hours. After cooling, excess thionyl chloride was removed in vacuo, and the resulting acid chloride (IAa.125) was dissolved in 5 ml of methylene chloride.

This solution of the acid chloride (IAa.125) was added dropwise to a solution of 0.16 g of methyl R-lactate in 10 ml of methylene chloride and 0.16 g of triethylamine, and the mixture was stirred for 8 hours with addition of a catalytic amount of DMAP. Removal of the solvent in vacuo and subsequent chromatography of the residue on silica gel with cyclohexane/ethyl acetate (95:5, v/v) yielded 0.47 g of the title compound (R enantiomer).

The experiment was repeated with the difference that the same amount of methyl S-lactate was employed instead of methyl R-lactate, yielding 0.42 g of the S enantiomer.

¹H NMR (CDCl₃): δ (ppm)=8.8 (S, 1H), 8.1 (d, 1H), 7.4 (d, 1H), 5.4 (q, 1H), 3.8 (s, 3H), 2.6 (s, 3H), 1.6 (d, 3H).

### Example 10

### Preparation of 2-(2,4-dichloro-5-methoxyphenyl)-fluoro-4-methyl-5-trifluoromethylpyridine (IAf.3)

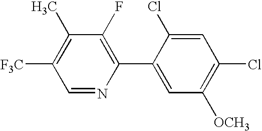

10.1 2-Benzyloxy-3-fluoro-5-trifluoromethylpyridine

The procedure as described in Example 1.1 was followed, and 13 g of 2-benzyloxy-3-fluoro-5-trifluoromethylpyridine were prepared starting from 9.9 g of 2,3-difluoro-5-trifluoromethylpyridine.

¹H NMR (CDCl₃): δ (ppm)=8.3 (s, 1H), 7.6 to 7.3 (m, 6H), 5.5 (s, 2H).

10.2 2-Benzyloxy-3-fluoro-4-methyl-5-trifluoromethylpyridine

8.1 g of 2-benzyloxy-3-fluoro-4-methyl-5-trifluoromethylpyridine were prepared starting from 9.4 g (36.5 mmol) of the pyridine of Example 10.1 analogously to the procedure described in Example 1.2.

¹H NMR (CDCl₃): δ (ppm)=8.1 (s, 1H), 7.5 to 7.3 (m, 5H), 5.5 (s, 2H), 2.4 (s, 3H).

10.3 2-Hydroxy-3-fluoro-4-methyl-5-trifluoromethylpyridine

3.8 g of 2-hydroxy-3-fluoro-4-methyl-5-trifluoromethylpyridine were prepared starting from 8.0 g (28.07 mmol) of the pyridine of Example 10.2 analogously to the procedure described in Example 1.3.

¹H NMR (CDCl₃): δ (ppm)=13.0 (br, 1H), 7.6 (s, 1H), 2.4 (s, 3H).

10.4 2-Chloro-3-fluoro-4-methyl-5-trifluoromethylpyridine

3.7 g of 2-chloro-3-fluoro-4-methyl-5-trifluoromethylpyridine were prepared starting from 3.8 g (19.5 mmol) of the pyridine of Example 10.3 analogously to the procedure described in Example 1.4.

¹H NMR (CDCl₃): δ (ppm)=8.4 (s, 1H), 2.5 (s, 3H).

10.5 2,4-Dichloro-5-methoxyphenylboronic Acid

20.8 g (6.8 mmol) of 2,4-dichloro-5-methoxyiodobenzene (CAS-No. 189138-40-7) were converted into the boronic acid with 36.4 ml (7.3 mmol) of an isopropylmagnesium chloride solution (2 M in ether) and 21.4 g of trimethyl borate analogously to the procedure described in Example 4.2. This gave 11.1 g of 2,4-dichloro-5-methoxyphenylboronic acid.

¹H NMR (d₆-DMSO): δ (ppm)=8.4 (br, 2H), 7.4 (s, 1H), 7.1 (s, 1H), 3.9 (s, 3H).

10.6 2-(2,4-Dichloro-5-methoxyphenyl-1-yl]-3-fluoro-4-methyl-5-trifluoromethylpyridine

This was prepared analogously to the procedure described in Example 1.5. Starting from 3.7 g (17.3 mmol) of the pyridine of Example 10.4 and 3.8 g (17.3 mmol) of the boronic acid of Example 10.5 in dimethoxyethane/water (4:1, v/v) in the presence of 0.8 mmol of [1,2-bis(diphenylphosphine)butane]palladium(II) chloride as catalyst, 2.8 g of 2-(2,4-dichloro-5-methoxyphenyl-1-yl]-3-fluoro-4-methyl-5-tri fluoromethylpyridine were obtained.

¹H NMR (CDCl₃): δ (ppm)=8.7 (s, 1H), 7.5 (s, 1H), 7.0 (s, 1H), 3.9 (s, 3H), 2.6 (s, 3H).

### Example 11

### Preparation of 2-(2-fluoro-4-cyano-5-methoxyphenyl]-3-chloro-4-methyl-5-trifluoromethylpyridine (IAd.3)

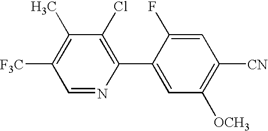

11.1 2-Fluoro-4-cyano-5-methoxyphenylboronic Acid

10 ml (20.3 mmol) of a solution of isopropylmagnesium chloride (2 M in ether) were added dropwise at −40° C. with stirring to 5.3 g (19.1 mmol) of 2-methoxy-4-iodo-5-fluorobenzonitrile (obtainable analogously to Example 4.1 from 2-fluoro-4-cyano-5-methoxyaniline) in 50 ml methyl tert-butyl ether and 20 ml of THF, and stirring was continued for 1 hour. 6.0 g (57.4 mmol) of trimethyl borate were subsequently added dropwise, stirring of the mixture was continued for 1 hour at −40° C. and the mixture was allowed to afterreact overnight at room temperature with stirring. The mixture was subsequently treated with 50 ml of saturated ammonium chloride solution, diluted with saturated sodium chloride solution and extracted three times with in each case 100 ml of ethyl acetate. After drying of the combined organic phases over sodium sulfate and concentrating the solution, the residue was digested in n-hexane and the solid was filtered off with suction. The mother liquor was subsequently diluted with ethyl acetate and extracted three times with 5% strength NaOH solution. The combined aqueous phases were acidified with 10% strength hydrochloric acid and subsequently extracted three more times with in each case 50 ml of ethyl acetate. In total, 1.6 g of a colorless solid of m.p. 213 to 214° C. were isolated. Depending on the work-up, it was possible that the product also contained the trimer boron oxine, which, however, reacted further like the desired boronic acid.

¹H NMR (d₆-DMSO): δ (ppm)=8.7 (br, 2H), 7.6 (d, 1H), 7.4 (d, 1H), 3.9 (s, 3H).

11.2 2-(2-Fluoro-4-cyano-5-methoxyphenyl-1-yl]-3-chloro-4-methyl-5-trifluoromethylpyridine

The title compound was prepared analogously to the procedure described in Example 1.5. Starting from 1.8 g (7.7 mol) of the pyridine of Example 1.4 and 1.5 g (7.7 mmol) of the cyanoboronic acid of Example 11.1, 1.0 g of the title compound of m.p. 108 to 109° C. were obtained.

¹H NMR (CDCl₃): δ (ppm)=8.8 (s, 1H), 7.4 (d, 1H), 7.0 (d, 1H), 4.0 (s, 3H), 2.6 (s, 3H).

### Example 12

### 2-[2-Fluoro-4-(methoxycarbonyl)methoxy-5-nitrophenyl]-3-chloro-4-methyl-5-trifluoromethylpyridine (Comp. Ias.5)

2-(2,4-Difluorophenyl)-3-chloro-4-methyl-5-trifluoromethylpyridine was prepared analogously to Example 1.5. This compound was nitrated anologously to the protocol of Example 5 yielding 2-(2,4-difluoro-5-nitrophenyl)-3-chloro-4-methyl-5-trifluormethyl pyridine. The nitro compound was then reacted with methyl glycolate in dioxane in the presence of potassium fluoride as base to give the title compound.

### Example 13

### 7-(3-Chloro-4-methyl-5-trifluoromethylpyridin-2-yl)-6-fluoro-2H-1,4-benzoxazin-3-one (IBa.1)

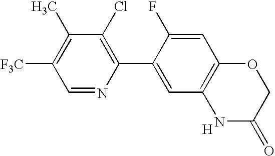

2.4 g (5.7 mmol) of the nitrophenyl ester Iaw.5 of Example 12 were dissolved in 150 ml of methanol, 1 g of Pt (5% on charcoal) was added, and the mixture was treated with 0.0171 mol H₂ (1 bar). The reaction mixture was subsequently filtered through kieselguhr in order to remove the catalyst and concentrated. The residue was taken up in 25 ml of DMF and 1.7 g (12.2 mmol) of K₂CO₃ were added. To complete the cyclization, the mixture was then stirred for 2 hours at 70° C. The mixture was then diluted with 150 ml of water and extracted three times with in each case 100 ml of methyl tert-butyl ether, and the combined organic phases were dried over sodium sulfate. Concentration gave 1.7 g of 7-(3-chloro-4-methyl-5-trifluoromethylpyridin-2-yl)-6-fluoro-2H-1,4-benzoxazin-3-one, which was directly reacted further.

1H NMR (CDCl₃): δ (ppm)=9.5 (br, 1H), 8.8 (s, 1H), 6.9 (d, 1H), 6.8 (d, 1H), 4.5 (s, 2H), 2.6 (s, 3H).

### Example 14

### 7-(3-Chloro-4-methyl-5-trifluoromethylpyridin-2-yl)-6-fluoro-4-propargyl-2H-1,4-benzoxazin-3-one (IBa.24)

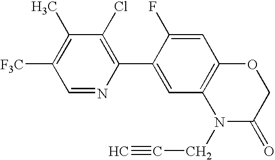

0.23 g (1.66 mmol) of potassium carbonate and then 0.18 g (1.5 mmol) of propargyl bromide were added to a solution of 0.5 g (1.4 mmol) of Example 13 in 10 ml of DMF. The mixture was stirred at room temperature until TLC revealed no further change. For work-up, the reaction mixture was poured into water and the product which precipitated was filtered off with suction. Washing of the residue with water gave 0.45 g of 7-(3-chloro-4-methyl-5-trifluoromethylpyridin-2-yl)-6-fluoro-4-propargyl-2H-1,4-benzoxazin-3-one of m.p. 156 to 157° C.

¹H NMR (CDCl₃): δ (ppm)=8.8 (s, 1H), 7.2 (d, 1H), 6.8 (d, 1H), 4.7 (m, 4H), 2.6 (s, 3H), 2.2 (t, 1H).

### Example 15

### 2-(2-Fluoro-4-chloro-5-azidophenyl)-3-chloro-4-methyl-5-trifluoromethylpyridine (IAa.84)

1.75 g (17.0 mmol) of tert-butyl nitrite were added dropwise at 5° C. to a solution of 5.5 g (16.2 mmol) of the aniline of Example 6 in 60 ml of trifluoroacetic acid. After 40 minutes at this temperature, 1.58 g (24.3 mmol) of sodium azide were added portionwise. Stirring was continued for 1 hour at 0 to 5° C. and for 2 hours at room temperature, and the reaction mixture was introduced into 500 ml of ice-cold water and extracted three times with in each case 200 ml of methylene chloride. The combined organic phases were washed twice with in each case 100 ml of water, once with 100 ml of 5% strength sodium hydroxide solution and again with 100 ml of water, dried over magnesium sulfate and concentrated. This gave 4.2 g of the title compound IAa.84

¹H NMR (CDCl₃): δ (ppm)=8.8 (s, 1H), 7.3 (m, 2H), 2.6 (s, 3H).

### Example 16

### 4-Chloro-7-[3-chloro-4-methyl-5-(trifluoromethyl)-2-pyridinyl]-2-ethyl-6-fluoro-1,3-benzoxazole (ICe.3)

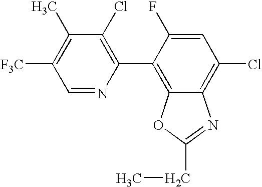

1.5 g of the azide of Example 15 were treated with 30 ml of propionic acid and the mixture was refluxed for 7 hours. The reaction mixture was subsequently poured into 200 ml of ice-cold water and neutralized with 5% strength sodium hydroxide solution. The mixture was extracted three times with in each case 100 ml of ethyl acetate, the combined organic phases were dried over sodium sulfate and the solvent was removed in vacuo. Chromatography on silica gel with cyclohexane/ethyl acetate (10/1, v/v) yielded 0.25 g of the title compound.

¹H NMR (CDCl₃): δ (ppm)=8.8 (s, 1H), 7.3 (s, 1H), 3.0 (q, 2H), 2.6 (s, 3H), 1.4 (t, 3H).

### Example 17

### 2-(2-Fluoro-4-chloro-5-methoxyphenyl-1-yl)-3-chloro-4-amino-5-trifluoromethylpyridine (IAi.3)

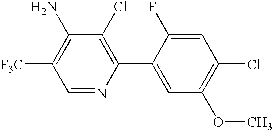

17.1 2-Benzyloxy-3-chloro-4-carboxy-5-trifluoromethylpyridine

41 ml (53.3 mmol) of a 1.3 M butyllithium solution in n-hexane was added dropwise −75° C. to a solution of 14.5 g (43.7 mmol) of 2-benzyloxy-3-chloro-5-trifluoromethylpyridine of Example 1.1 in approximately 200 ml of THF and stirring was continued at this temperature for 1 hour. This solution was then added dropwise at −75° C. to 100 ml of a saturated solution of carbon dioxide in THF. After the addition had ended, carbon dioxide was passed in within 1 hour. The mixture was defrosted to −10° C., 100 ml of saturated ammonium chloride solution were added, the mixture was diluted with saturated sodium chloride solution, and the organic phase was subsequently separated off. The aqueous phase was then extracted twice more with in each case approximately 200 ml of methyl tert-butyl ether and the combined organic phases were washed with water. After drying of the organic phase over sodium sulfate and concentrating the solution, 14.7 g of 2-benzyloxy-3-chloro-4-carboxy-5-trifluoromethylpyridine were obtained.

¹H NMR (d₆-DMSO): δ (ppm)=8.3 (s, 1H), 7.4 to 7.2 (m, 5H), 5.5 (s, 2H).

17.2 2-Benzyloxy-3-chloro-4-(N-tert-butoxycarbonyl)amino-5-trifluoromethylpyridine

4.63 g (45.9 mmol) of triethylamine and 12.0 g (43.7 mmol) of diphenylphosphoryl azide were added to 14.5 g (43.7 mmol) of the acid of Example 17.1 in 180 ml tert-butanol, and the mixture was stirred for 10 hours at room temperature. The mixture was subsequently concentrated and the residue was chromatographed on silica gel using cyclohexane/ethyl acetate. This gave 10.3 g of 2-benzyloxy-3-chloro-4-(N-tert-butoxycarbonyl)amino-5-trifluoromethylpyridine.

¹H NMR (CDCl₃): δ (ppm)=9.3 (br, 1H), 8.5 (s, 1H), 7.5 to 7.3 (m, 5H), 5.5 (s, 2H), 1.4 (s, 9H).

17.3 2-Hydroxy-3-chloro-4-amino-5-trifluoromethylpyridine

7.2 g (66.3 mmol) of trimethylsilyl chloride were added dropwise to 9.9 g of sodium iodide in 120 ml of acetonitrile. After 20 minutes, a solution of 10.7 g of the amide of Example 9.2 in 80 ml of acetonitrile was added. The reaction mixture was stirred for 2 hours at 50° C. The mixture was subsequently concentrated in vacuo, the residue was introduced into ice-cold water, and the mixture was extracted three times with in each case 200 ml of ethyl acetate. The aqeuous phase was brought to pH 7 using 5% strength sodium hydroxide solution and reextracted twice with ethyl acetate. The combined organic phases were subsequently washed with 100 ml of water. After drying of the organic phase over sodium sulfate and concentration, chromatography of the crude product on silica gel using cyclohexane/ethyl acetate (gradient 5/1 to 1/2, v/v) yielded 4.5 g of 2-hydroxy-3-chloro-4-amino-5-trifluoromethylpyridine.

¹H NMR (CDCl₃): δ (ppm)=11.5 (br, 1H), 7.6 (s, 1H), 6.4 (s, 2H).

7.4 2,3-Dichloro-4-amino-5-trifluoromethylpyridine

3.6 g (16.9 mmol) of the hydroxypyridine of Example 17.3 was stirred for 2 hours at 75° C. with 50 ml of phosphoryl chloride, excess phosphoryl chloride was removed on a rotary evaporator, and the residue was treated with water. The mixture was subsequently extracted three times with in each case 50 ml of methylene chloride, the organic phase was dried over sodium sulfate, and the solvent was removed in vacuo.

This gave 3.0 g of 2,3-dichloro-4-amino-5-trifluoromethylpyridine.

¹H NMR (d₆-DMSO): δ (ppm)=8.2 (s, 1H), 7.2 (s, 2H).

17.5 2-(2-Fluoro-4-chloro-5-methoxyphenyl-1-yl)-3-chloro-4-amino-5-trifluoromethylpyridine

Analogously to the procedure described in Example 1.5, 2.0 g (8.7 mmol) of the aminochloropyridine of Example 17.4 were treated with 2-fluoro-4-chloro-5-methoxyphenylboronic acid. Chromatography of the crude product on silica gel with cyclohexane/ethyl acetate (15/1, v/v) gave 1.0 g of the title compound.

¹H NMR (d₆-DMSO): δ (ppm)=8.4 (s, 1H), 7.6 (d, 2H), 7.2 (d, 2H), 3.9 (s, 3H). The NH₂ signal is located broadly under the H₂O signal.

The compounds of the following Examples 19 to 77 were prepared analogously.

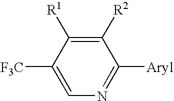

| Ex. | R¹ | R² | Aryl | ¹H NMR | m.p. |
|---|---|---|---|---|---|
| 19 | CH₃ | Cl | 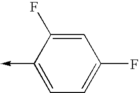 | (CDCl₃) 8.8 (s, 1 H), 7.4 (m, 1 H), 6.8 (m, 2 H), 2.6 (s, 3 H), | 38 to 40° C. |
| 20 | CH₃ | Cl | 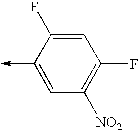 | (CDCl₃) 8.8 (s, 1 H), 8.3 (m, 1 H), 7.2 (m, 1H), 2.6 (s, 3 H). | 92 to 93° C. |
| 21 | CH₃ | Cl | 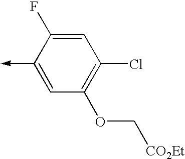 | (CDCl₃) 8.8 (s, 1 H), 7.3 (d, 1 H), 7.0 (d, 1 H), 4.7 (s, 2 H), 4.2 (q, 2 H), 2.6 (s, 3 H), 1.3 (t, 3 H). | 72 to 73° C. |
| 22 | CH₃ | Cl | 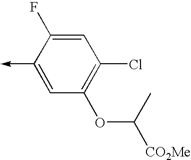 | (CDCl₃) 8.8 (s, 1 H), 7.2 (d, 1 H), 7.0 (d, 1 H), 4.8 (q, 1 H), 3.8 (s, 3 H), 2.6 (s, 3 H), 1.7 (d, 3 H). | |
| 23 | CH₃ | Cl | 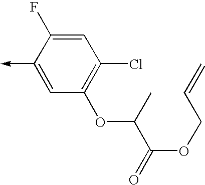 | (CDCl₃) 8.8 (s, 1 H), 7.2 (d, 1 H), 7.0 (d, 1 H), 5.8 to 5.1 (m, 2 H), 4.8 (q, 1 H), 4.6 (m, 2 H), 2.6 (s, 3 H), 1.7 (d, 3 H). | |
| 24 | CH₃ | Cl | 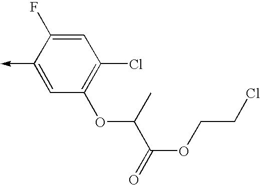 | (CDCl₃) 8.8 (s, 1 H), 7.2 (d, 1 H), 7.0 (d, 1 H), 4.8 (q, 1 H), 4.2 (m, 2 H), 3.7 (m, 2 H), 2.6 (s, 3 H), 1.7 (d, 3 H). | |
| 25 | CH₃ | Cl | 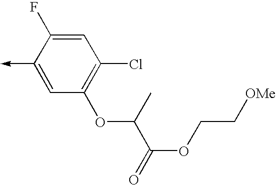 | (CDCl₃) 8.8 (s, 1 H), 7.2 (d, 1 H), 7.0 (d, 1 H), 4.8 (q, 1 H), 4.3 (m, 2 H), 3.4 (m, 2 H), 3.3 (s, 3 H) 2.6 (s, 3 H), 1.7 (d, 3 H) | |
| 26 | CH₃ | Cl | 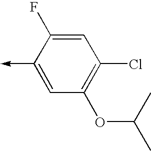 | (CDCl₃) 8.8 (s, 1 H), 7.2 (d, 1 H), 7.0 (d, 1 H), 4.5 (sept, 1 H), 2.6 (s, 3H), 1.4 (d, 6 H) | |
| 27 | CH₃ | Cl | 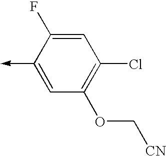 | (CDCl₃) 8.8 (s, 1 H), 7.3 (d, 1 H), 7.1 (d, 1 H), 4.8 (s, 2 H), 2.6 (8, 3H). | 136 to 137° C. |
| 28 | CH₃ | Cl | 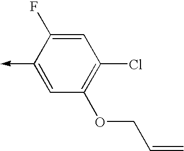 | (CDCl₃) 8.8 (s, 1 H), 7.2 (d, 1 H), 7,0 (d, 1 H), 6.0 (m, 1 H), 5.5 to 5.3 (m, 2 H), 4.6 (m, 2 H), 2.6 (s, 3 H). | 79 to 80° C. |
| 29 | CH₃ | Cl | 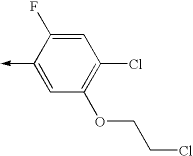 | (CDCl₃) 8.8 (s, 1 H), 7.2 (d, 1 H), 7.0 (d, 1 H), 4.3 (m, 2 H), 3.9 (m, 2 H), 2.6 (s, 3 H). | 97 to 98° C. |
| 30 | CH₃ | Cl | 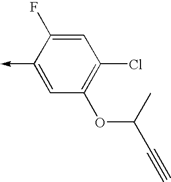 | (CDCl₃) 8.8 (s, 1 H), 7.3 to 7.1 (m, 2 H), 4.9 (m, 1 H), 2.6 (s, 3 H), 2.5 (s, 1 H), 1.8 (d, 3 H). | 71 to 73° C. |
| 31 | CH₃ | Cl | 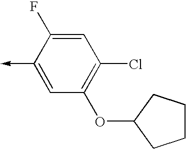 | (CDCl₃) 8.8 (s, 1 H), 7.4 (d, 1 H), 7.0 (d, 1 H), 4.8 (m, 1 H), 2.6 (s, 3 H), 2.0 to 1.6 (m, 8 H). | |
| 32 | CH₃ | Cl | 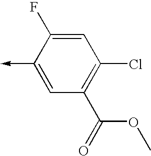 | (CDCl₃) 8.8 (s, 1 H), 8.0 (d, 1 H), 7.4 (d, 1 H), 3.9 (m, 2 H), 2.6 (s, 3 H). | 150 to 152° C. |
| 33 | CH₃ | Cl | 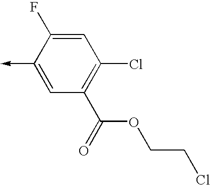 | (CDCl₃) 8.8 (s, 1 H), 8.1 (d, 1 H), 7.4 (d, 1 H), 4.6 (m, 2 H), 3.8 (m, 2 H), 2.6 (s, 3 H). | |
| 34 | CH₃ | Cl | 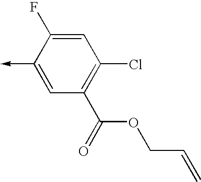 | (CDCl₃) 8.8 (s, 1 H), 8.1 (d, 1 H), 7.4 (d, 1 H), 6.0 (m, 1 H), 5.5 to 5.2 (m, 2 H), 4.8 (d, 2 H), 2.6 (5, 3 H). | |
| 35 | CH₃ | Cl | 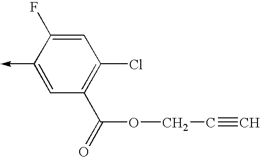 | (CDCl₃) 8.8 (s, 1 H), 8.1 (d, 1 H), 7.4 (d, 1 H), 4.9 (d, 2 H), 2.6 (s, 3 H), 2.5 (t, 1 H). | |
| 36 | CH₃ | Cl | 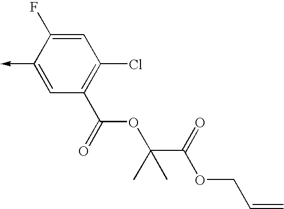 | (CDCl₃) 8.8 (s, 1 H), 8.0 (d, 1 H), 7.4 (d, 1 H), 5.9 (mc, 1 H), 5.4 to 5.2 (m, 2 H), 4.6 (m, 2 H), 2.6 (s, 3 H), 1.7 (s, 6 H). | |
| 37 | CH₃ | Cl | 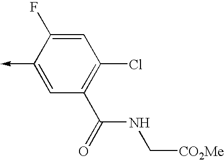 | (CDCl₃) 8.8 (s, 1 H), 7.9 (d, 1 H), 7.3 (d, 1 H), 6.9 (br, 1 H), 4.3 (d, 2 H), 3.8 (s, 3 H), 2.6 (s, 3 H), 2.5 (t, 1 H). | 104 to 105° C. |
| 38 | CH₃ | Cl | 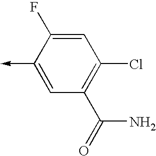 | (CDCl₃) 8.8 (s, 1 H), 8.0 (d, 1 H), 7.3 (d, 1 H), 6.5 to 6.0 (br, 2 H), 2.6 (s, 3 H), 2.5 (t, 1 H). | 183 to 184° C. |
| 39 | CH₃ | Cl | 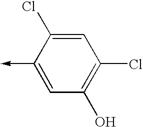 | (CDCl₃) 8.8 (s, 1 H), 7.4 (s, 1 H), 6.9 (s, 1 H), 4.6 (m, 2 H), 2.6 (s, 3H). | 201 to 203° C. |
| 40 | CH₃ | Cl | 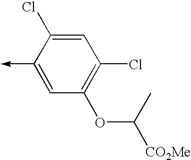 | (CDCl₃) 8.8 (s, 1 H), 7.4 (s, 1 H), 6.8 (s, 1 H), 4.8 (q, 1 H), 3.8 (s, 3 H), 2.6 (s, 3 H), 1.7 (d, 3 H). | |
| 41 | CH₃ | Cl | 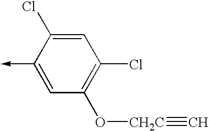 | (CDCl₃) 8.8 (s, 1 H), 7.5 (s, 1 H), 7.0 (s, 1 H), 4.8 (d, 2 H), 2.6 (t, 1H), 2.5 (s 3 H). | 95 to 96° C. |
| 42 | CH₃ | F | 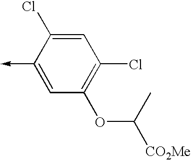 | (CDCl₃) 8.7 (s, 1 H), 7.5 (s, 1 H), 6.9 (s, 1 H), 4.8 (q, 1 H), 3.8 (s, 3 H), 2.5 (s, 3 H), 1.7 (d, 3 H). | |
| 43 | CH₃ | F | 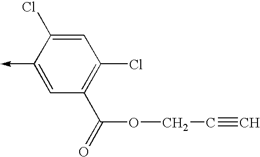 | (CDCl₃) 8.7 (s, 1 H), 7.57 (s, 1 H), 7.0 (s, 1 H), 4.8 (d, 2 H), 2.6 (t, 1 H), 2.5 (s, 3 H). | 69 to 70° C. |
| 44 | CH₃ | F | 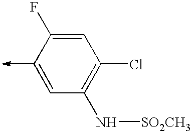 | (CDCl₃) 8.8 (s, 1 H), 7.7 (d, 1 H), 7.3 (d, 1 H), 7.1 (br, 1 H), 3.0 (s, 3 H), 2.6 (t, 1 H), 2.5 (s, 3 H). | 138 to 139° C. |
| 45 | CH₃ | F | 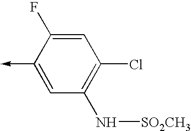 | (CDCl₃) 8.8 (s, 1 H), 7.7 (d, 1 H), 7.6 (br, 1 H), 7.3 (d, 1 H), 3.2 (q, 2 H), 2.6 (t, 1 H), 2.5 (s, 3 H), 1.4 (t, 3 H). | 142 to 146° C. |
| 46 | CH₃ | F | 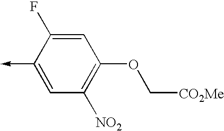 | (CDCl₃) 8.8 (s, 1 H), 8.1 (d, 1 H), 6.8 (d, 1 H), 4.9 (s, 1 H), 3.8 (s, 3 H), 2.6 (s, 3 H). | |
| 47 | CH₃ | F | 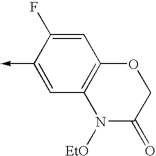 | (CDCl₃) 8.8 (s, 1 H), 7.2 (d, 1 H), 6.8 (d, 1 H), 4.8 (s, 2 H), 4.2 (q, 2 H), 2.6 (s, 3 H), 1.4 (t, 3 H). | 107 to 109° C. |
| 48 | CH₃ | F | 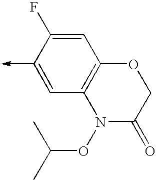 | (CDCl₃) 8.8 (s, 1 H), 7.2 (d, 1 H), 6.8 (d, 1 H), 4.8 (s, 2 H), 4.5 (sept, 1 H), 2.6 (s, 3 H), 1.3 (d, 6 H). | 113 to 114° C. |
| 49 | CH₃ | F | 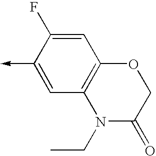 | (CDCl₃) 8.8 (s, 1 H), 7.0 (d, 1 H), 6.8 (d, 1 H), 4.5 (s, 2 H), 4.0 (q, 2 H), 2.6 (s, 3 H), 1.4 (t, 3 H). | 127 to 128° C. |
| 50 | CH₃ | F | 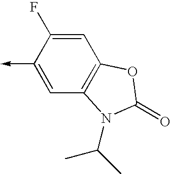 | (CDCl₃) 8.8 (s, 1 H), 7.1 (d, 1 H), 6.9 (d, 1 H), 4.5 (sept, 1 H), 2.6 (s, 3 H), 1.6 (d, 6 H). | |
| 51 | CH₃ | F | 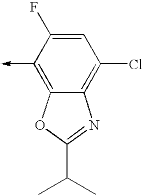 | (CDCl₃) 8.8 (s, 1 H), 7.2 (d, 1 H), 3.2 (sept, 1 H), 2.6 (s, 3 H), 1.4 (d, 6 H). | |
| 52 | CH₃ | F | 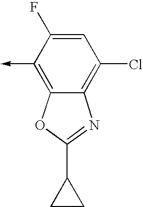 | (CDCl₃) 8.8 (s, 1 H), 7.2 (d, 1 H), 6.9 (d, 1 H), 2.6 (s, 3 H), 2.2 (m 1, 1.4 to 1.2 (m, 4 H). | |
| 53 | CH₃ | Cl | 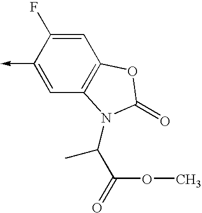 | (CDCl₃) 8.8 (s, 1 H), 7.2 (d, 1 H), 7.0 (d, 1 H), 5.1 (q, 1 H), 3.8 (s, 3 H), 2.6 (s, 3H), 1.8 (d, 2 H). | |
| 54 | CH₃ | Cl | 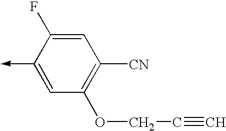 | (d₆-DMSO) 8.8 (s, 1 H), 7.5 (d, 1 H), 7,2 (d, 1 H), 4.8 (s, 1 H), 2.7-2.6 (m, 4H). | |
| 55 | NH₂ | Cl | 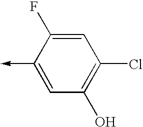 | (d₆-DMSO) 8.6 (s, 1 H), 8.1 (br, 1 H), 7.6 (d, 1 H), 7.1 (d, 1 H), NH₂ broad | |
| 56 | NH₂ | Cl | 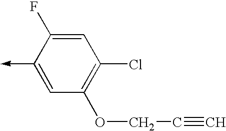 | (d₆-DMSO) 8.4 (s, 1 H), 7.6 (d, 1 H), 7.3 (d, 1 H), 7.0 (br, 2 H), 4.9 (d, 2 H), 3.6 (t, 1 H). | 115 to 118° C. |
| 57 | NH₂ | Cl | 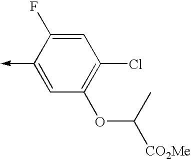 | (CDCl₃) 8.5 (s, 1 H), 7.3 (d, 1 H), 7.0 (d, 1 H), 5.4 (br, 2 H), 4.7 (q, 2 H), 3.8 (s, 3 H), 1.7 (d, 3 H). | |
| 58 | CH₃ | Cl | 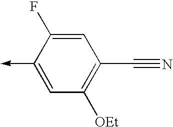 | (CDCl₃) 8.8 (s, 1 H), 7.4 (d, 1 H), 7.0 (d, 1 H), 4.2 (q, 2 H), 2.6 (s, 3 H), 1.5 (t, 3 H). | |
| 59 | CH₃ | Cl | 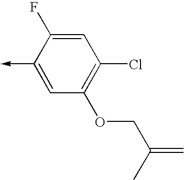 | (CDCl₃) 8.8 (s, 1 H), 7.3 (d, 1 H), 7.0 (d, 1 H), 5.2 (s, 1 H), 5.1 (s, 1 H), 4.4 (s, 2 H), 2.6 (s, 3 H). | |
| 60 | CH₃ | Cl | 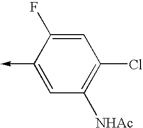 | (CDCl₃) 8.8 (s, 1 H), 8.5 (d, 1 H), 7.6 (br. s., 1 H), 7.2 (d, 1 H), 2.6 (s, 3 H), 2.2 (s, 3 H). | |
| 61 | CH₃ | Cl | 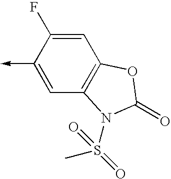 | (CDCl₃) 8.8 (s, 1 H), 7.3 (d, 1 H), 7.0 (d, 1 H). | |
| 62 | CH₃ | Cl | 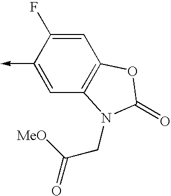 | (CDCl₃) 8.8 (s, 1 H), 7.2 (d, 1 H), 7.0 (d, 1 H), 4.6 (s 2 H), 3.8 (s, 3 H), 2.6 (s, 3 H). | |
| 63 | CH₃ | Cl | 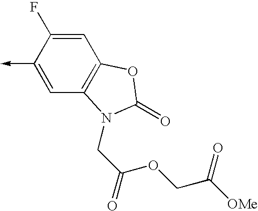 | (CDCl₃) 8.8 (s, 1 H), 7.2 (d, 1 H), 7.0 (d, 1 H), 5.2 (q 1 H), 4.7 (m, 2 H), 3.7 (s, 3 H), 2.6 (s, 3 H), 1.3 (d, 3 H). | |
| 64 | CH₃ | Cl | 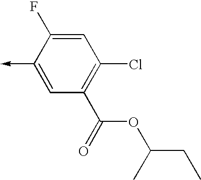 | (CDCl₃) 8.8 (s, 1 H), 8.0 (d, 1 H), 7.4 (d, 1 H), 5.2 (m 1 H), 2.6 (6, 3 H), 1.7 (d 3 H), 1.8 to 1.6 (m, 1 H), 1.4 (d, 3 H), 1.0 (t, 3 H). | |
| 65 | CH₃ | Cl | 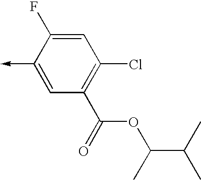 | (CDCl₃) 8.8 (s, 1 H), 8.0 (d, 1 H), 7.4 (d, 1 H), 5.0 (m, 1 H), 2.6 (s, 3 H), 2.0 (m, 1 H), 1.3 (d, 3 H), 1.0 (d, 6 H). | |
| 66 | CH₃ | Cl | 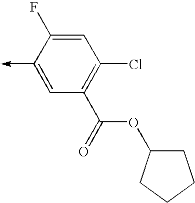 | (CDCl₃) 8.8 (s, 1 H), 8.0 (d, 1 H), 7.4 (d, 1 H), 5.4 (m, 1 H), 2.6 (s, 3 H), 2.0 to 1.5 (m 8 H). | |
| 67 | CH₃ | Cl | 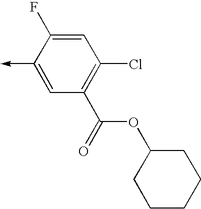 | (CDCl₃) 8.8 (s, 1 H), 8.0 (d, 1 H), 7.4 (d, 1 H), 5.1 (m, 1 H), 2.6 (s, 3 H), 2.0 to 1.5 (m, 10 H). | |
| 68 | CH₃ | Cl | 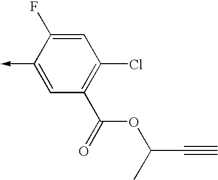 | (CDCl₃) 8.8 (s, 1 H), 8.0 (d, 1 H), 7.4 (d, 1 H), 6.7 (m, 1 H), 2.6 (s, 3 H), 2.5 (s, 1 H), 1.6 (d, 3 H). | |
| 69 | CH₃ | Cl | 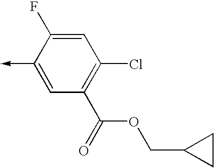 | (CDCl₃) 8.8 (s, 1 H), 8.0 (d, 1 H), 7.4 (d, 1 H), 4.2 (d 2 H), 2.6 (s, 3 H), 1.3 (m, 1 H), 1.6 to 1.4 (m, 4 H). | |
| 70 | CH₃ | Cl | 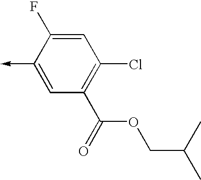 | (CDCl₃) 8.8 (s, 1 H), 8.0 (d, 1 H), 7.4 (d, 1 H), 4.1 (d 2 H), 2.6 (s, 3 H), 1.3 (m, 1 H), 2.1 (m, 1 H), 1.0 (d, 6 H). | |
| 71 | CH₃ | Cl | 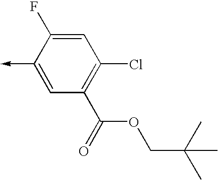 | (CDCl₃) 8.8 (s, 1 H), 8.0 (d, 1 H), 7.4 (d, 1 H), 4.0 (s, 2 H), 2.6 (s, 3 H), 1.0 (s, 9 H). | |
| 72 | CH₃ | Cl | 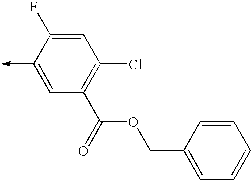 | (CDCl₃) 8.8 (s, 1 H), 8.1 (d, 1 H), 7.5 to 7.2 (m, 6 H), 5.4 (s 2 H), 2.6 (s, 3 H). | |
| 73 | CH₃ | Cl | 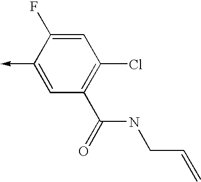 | (CDCl₃) 8.8 (s, 1 H), 7.8 (d, 1 H), 7.3 (d, 1 H), 6.4 (br. s, 1 H), 6.0 (m, 1 H), 5.4 to 5.2 (m, 2 H), 4.1 (m, 2 H), 2.6 (s, 3 H), | |
| 74 | CH₃ | Cl | 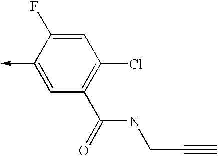 | (CDCl₃) 8.8 (s, 1 H), 7.8 (d, 1 H), 7.3 (d, 1 H), 6.5 (br. s. 1 H), 4.3 (m, 2 H), 2.6 (s, 3 H), 2.3 (m, 1 H). | |
| 75 | CH₃ | Cl | 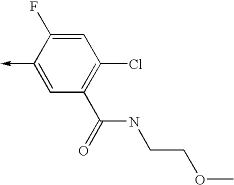 | (CDCl₃) 8.8 (s, 1 H), 7.8 (d, 1 H), 7.4 (d, 1 H), 5.9 (mc, 1 H), 5.4 to 5.2 (m, 2 H), 4.6 (m, 2 H), 2.6 (s, 3 H), 1.7 (s, 6 H). | |
| 76 | CH₃ | Cl | 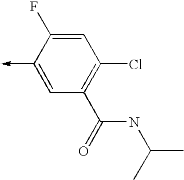 | (CDCl₃) 8.8 (s, 1 H), 7.8 (d, 1 H), 7.4 (d, 1 H), 6.1 (br. d, 1 H), 4.3 (m, 1 H), 2.6 (s, 3 H), 1.3 (d, 6 H). | |
| 77 | CH₃ | Cl | 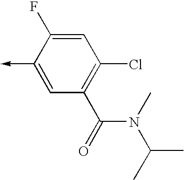 | (CDCl₃) rotamer mixture: 8.8 (s, 1 H), 7.8 (d, 1 H), 7.3 (d, 1 H), 5.0 (m, 0.5 H), 4.8 (m, 0.5 H), 3.0 (s, 1.5 H), 2.8 (s, 1.5 H), 2.6 (s, 3 H), 1.2 (m, 6 H). | |

The compounds I and their agriculturally useful salts, not only as isomer mixtures, but also in the form of the pure isomers, are suitable as herbicides. The herbicidal compositions comprising I effect very good control of vegetation on noncrop areas, especially at high application rates. In crops such as wheat, rice, maize, soybeans and cotton, they act against broad-leaved weeds and grass weeds without substantially harming the crop plants. This effect is observed especially at low application rates.

Depending on the application method in question, the compounds I or compositions comprising them can also be employed in a further number of crop plants for eliminating undesired plants. Examples of suitable crops are the following:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (S. vulgare), Theobroma cacao, Trifolium pratense, Triticum aestivum, Triticum durum, Vicia faba, Vitis vinifera, Zea mays.

In addition, the compounds I can also be used in crops which tolerate the effect of herbicides owing to breeding, including recombinant methods.

Moreover, the 3-halo-2-phenylpyridines and their agriculturally useful salts are also suitable for the desiccation and/or defoliation of plants.

As desiccants, they are suitable in particular for desiccating the aerial parts of crop plants such as potato, oilseed rape, sunflower and soybeans. This makes possible the full mechanization of the harvest of these important crop plants.

Also of economic interest are:

the dehiscence of fruit concentrated over a period of time, or the reduction in their adherence to the plant, for example in the case of citrus fruit, olives or other species and varieties of pomaceous fruit, stone fruit and hard-shelled fruit, since this facilitates the harvest of these fruits, and

the controlled removal of the foliage of useful plants, in particular cotton (defoliation).

The dehiscence which is promoted by the use of compounds of the formula I according to the invention and their agriculturally useful salts is based on the formation of abscission tissue between the fruit organ or leaf organ and the shoot organ of the plants. The defoliation of cotton is of very particular economic interest since it facilitates harvesting. At the same time, the shortening of the period of time within which the individual plants mature leads to an increased quality of the harvested fiber material.

The compounds I or the compositions comprising them can be applied for example in the form of directly sprayable aqueous solutions, powders, suspensions, also highly-concentrated aqueous, oily or other suspensions or dispersions, emulsions, oil dispersions, pastes, dusts, materials for spreading or granules by means of spraying, atomizing, dusting, spreading, pouring or treating the seed or mixing with the seed. The use forms depend on the intended purpose; in any case, they should guarantee the finest possible distribution of the active ingredients according to the invention. The herbicidal compositions comprise a herbicidally active amount of at least one compound of the formula I or of an agriculturally useful salt of I and auxiliaries conventional in the formulation of crop protection products.

Inert additives which are suitable are essentially the following:

Mineral oil fractions of medium to high boiling point, such as kerosene or diesel oil, furthermore coal tar oils and oils of vegetable or animal origin, aliphatic, cyclic and aromatic hydrocarbons, for example paraffin, tetrahydronaphthalene, alkylated napthalenes or their derivatives, alkylated benzenes or their derivatives, alcohols such as methanol, ethanol, propanol, butanol, cyclohexanol, ketones such as cyclohexanone, or strongly polar solvents, for example amines such as N-methylpyrrolidone, or water.

Aqueous use forms can be prepared from emulsion concentrates, suspensions, pastes, wettable powders or water-dispersible granules by adding water. To prepare emulsions, pastes or oil dispersions, the 3-halo-2-phenylpyridines, as such or dissolved in an oil or solvent, can be homogenized in water by means of wetter, adhesive, dispersant or emulsifier. However, it is also possible to prepare concentrates composed of active substance, wetter, adhesive, dispersant or emulsifier and, if appropriate, solvent or oil, which concentrates are suitable for dilution with water.

Suitable surface-active substances are the alkali metal salts, alkaline earth metal salts, ammonium salts of aromatic sulfonic acids, for example lignosulfonic acid, phenolsulfonic acid, naphthalenesulfonic acid and dibutylnaphthalenesulfonic acid, and of fatty acids, alkylsulfonates and alkylarylsulfonates, of alkyl sulfates, lauryl ether sulfates and fatty alcohol sulfates, and salts of sulfated hexa-, hepta- and octadecanols and of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl polyglycol ethers, tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignin-sulfite waste liquors or methylcellulose.

Powders, materials for spreading and dusts can be prepared by mixing or concomitantly grinding the active substances together with a solid carrier.

Granules, for example coated granules, impregnated granules, and homogeneous granules can be prepared by binding the active ingredients to solid carriers. Solid carriers are mineral earths such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas, and products of vegetable origin such as cereal meal, tree bark meal, wood meal and nutshell meal, cellulose powders or other solid carriers.

The concentrations of the active ingredients I in the ready-to-use preparations can be varied within wide ranges. In general, the formulations comprise 0.001 to 98% by weight, preferably 0.01 to 95% by weight, of at least one active ingredient. In this context, the active ingredients are employed in a purity of from 90% to 100%, preferably 95% to 100% (according to NMR spectrum).

For example, the compounds I according to the invention can be formulated as follows:

I 20 parts by weight of the compound No. IAa.3 are dissolved in a mixture composed of 80 parts by weight of alkylated benzene, 10 parts by weight of the adduct of 8 to 10 mol of ethylene oxide and 1 mol of oleic acid N-monoethanolamide, 5 parts by weight of calcium dodecylbenzenesulfonate and 5 parts by weight of the adduct of 40 mol of ethylene oxide and 1 mol of castor oil. Pouring the solution into 100,000 parts by weight of water and finely distributing it therein gives an aqueous dispersion comprising 0.02% by weight of the active ingredient.

II 20 parts by weight of the compound No. IAa.10 are dissolved in a mixture composed of 40 parts by weight of cyclohexanone, 30 parts by weight of isobutanol, 20 parts by weight of the adduct of 7 mol of ethylene oxide and 1 mol of isooctylphenol and 10 parts by weight of the adduct of 40 mol of ethylene oxide and 1 mol of castor oil. Pouring the solution into 100,000 parts by weight of water and finely distributing it therein gives an aqueous dispersion comprising 0.02% by weight of the active ingredient.

III 20 parts by weight of the active ingredient No. IAa.131 are dissolved in a mixture composed of 25 parts by weight of cyclohexanone, 65 parts by weight of a mineral oil fraction of boiling point 210 to 280° C. and 10 parts by weight of the adduct of 40 mol of ethylene oxide and 1 mol of castor oil. Pouring the solution into 100,000 parts by weight of water and finely distributing it therein gives an aqueous dispersion comprising 0.02% by weight of the active ingredient.

IV 20 parts by weight of the active ingredient No. IAa.143 are mixed thoroughly with 3 parts by weight of sodium diisobutylnaphthalenesulfonate, 17 parts by weight of the sodium salt of a lignosulfonic acid from a sulfite waste liquor and 60 parts by weight of pulverulent silica gel, and the mixture is ground in a hammer mill. Finely distributing the mixture in 20,000 parts by weight of water gives a spray mixture comprising 0.1% by weight of the active ingredient.

V 3 parts by weight of the active ingredient No. IAi.10 are mixed with 97 parts by weight of finely divided kaolin. This gives a dust comprising 3% by weight of the active ingredient.

VI 20 parts by weight of the active ingredient No. IBa.24 are mixed intimately with 2 parts by weight of calcium dodecylbenzenesulfonate, 8 parts by weight of fatty alcohol polyglycol ether, 2 parts by weight of the sodium salt of a phenol/urea/formaldehyde condensate and 68 parts by weight of a paraffinic mineral oil. This gives a stable oily dispersion.

VII 1 part by weight of the compound No. IBa.11 is dissolved in a mixture composed of 70 parts by weight of cyclohexanone, 20 parts by weight of ethoxylated isooctylphenol and 10 parts by weight of ethoxylated castor oil. This gives a stable emulsion concentrate.

VIII 1 part by weight of the compound No. ICe.3 is dissolved in a mixture composed of 80 parts by weight of cyclohexanone and 20 parts by weight of Wettol® EM 31 (nonionic emulsifier based on ethoxylated castor oil). This gives a stable emulsion concentrate.

The application of the herbicidal compositions or of the active ingredients can be effect pre-emergence, post-emergence or together with the seed of a crop plant. There is also the possibility of applying the herbicidal compositions or active ingredients by sowing the seed, of a crop plant, which has been pretreated with the herbicidal compositions or active ingredients. If the active ingredients are less well tolerated by specific crop plants, application techniques can be used in which the herbicidal compositions are sprayed with the aid of the spraying apparatus in such a way that the leaves of the sensitive crop plants come into as little contact as possible with the active ingredients, while these reach the leaves of undesired plants growing underneath the crop plants, or the naked soil (post-directed, lay-by).

Depending on the intended aim, the season, the target plants and the growth stage, the application rates of active ingredient are from 0.001 to 3.0, preferably 0.01 to 1.0 kg/ha of active substance (a.s.) per ha.

To widen the spectrum of action and to achieve synergistic effects, the 3-halo-2-phenylpyridines can be mixed, and applied jointly, with numerous representatives of other groups of herbicidally or growth-regulatory active ingredients. Examples of suitable components in mixtures are 1,2,4-thiadiazoles, 1,3,4-thiadiazoles, amides, aminophosphoric acid and its derivatives, aminotriazoles, anilides, (het)aryloxyalkanoic acids and their derivatives, benzoic acid and its derivatives, benzothiadiazinones, 2-aroyl-1,3-cyclohexanediones, 2-hetaroyl-1,3-cyclohexanediones, hetaryl aryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- or heteroaryloxyphenoxypropionic esters, phenylacetic acid and its derivatives, phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides, uracils.

Moreover, it may be advantageous to employ the compounds I, alone or in combination with other herbicides, as a mixture with yet further crop protection agents, for example with agents for controlling pests or phytopathogenic fungi or bacteria. Also of interest is the miscibility with mineral salt solutions which are employed for alleviating nutritional and trace element deficiencies. Nonphytotoxic oils and oil concentrates may also be added.

### USE EXAMPLES

The herbicidal action of the 3-halo-5-trifluoromethyl-2-phenylpyridines of the formula I was demonstrated by greenhouse 5 experiments:

The culture containers used were plastic pots with loamy sand with approximately 3.0% humus as substrate. The seeds of the test plants were sown separately for each species.

In the case of the pre-emergence treatment, the active ingredients which were suspended or emulsified in water were applied directly after sowing by means of finely distributing nozzles. The containers were irrigated gently to promote germination and growth and subsequently covered with translucent plastic hoods until the plants had rooted. This cover causes uniform germination of the test plants provided this was not adversely affected by the active ingredients.

For the purposes of the post-emergence treatment, the test plants were first grown to a height of 3 to 15 cm, depending on the growth habit, and only then treated with the active ingredients which were suspended or emulsified in water. To this end, the test plants were either sown directly and grown on in the same containers, or else they were first grown separately as seedlings and then transplanted into the experimental containers a few days prior to treatment. The application rate for the post-emergence treatment was 31.3, 15.6, 7.8 and/or 3.9 g of a.s./ha.

The plants were kept at temperatures of 10-25° C. or 20-35° C., depending on the species. The experimental period extended over 2 to 4 weeks. During this time, the plants were tended, and their response to the individual regimes were evaluated.

For the evaluation, a scale of 0 to 100 was used. 100 means no emergence of the plants, or complete destruction of at least the aerial parts, and 0 means no damage or normal course of growth.

The plants used in the greenhouse experiments consisted of the following species:

| Bayercode | Common name |
|---|---|
| ECHCG | barnyardgrass |
| SETFA | giant foxtail |
| BIDPI | hairy beggarticks |
| CHEAL | lambsquarters |
| BRAPL | alexandergrass |

The compounds I according to the invention which were tested were No. IAa.3 (Example 1) and IAa.10 (Example 3) and the corresponding compounds No. 1.501 (Comparative Example VA) and 1.512 (Comparative Example VB) of WO 95/02580.

Other compounds I according to the invention which were tested were No. ICe.3 (Example 16) and IBa.24 (Example 14) and the corresponding compounds No. Iz.003 (Comparative Example VC) of WO 99/06394 and the compound No. Ih.005 (Comparative Example VD) of WO 95/02590.

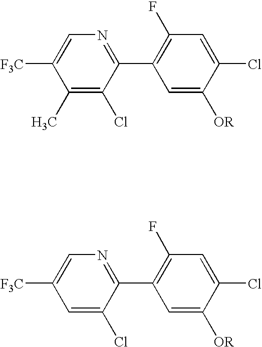

Compound according Comparative Example to the invention

| | | |
|---|---|---|
| R = CH₃ | No. IAa.3, | VA |
| R = CH₂C≡CH | No. IAa.10, | VB |

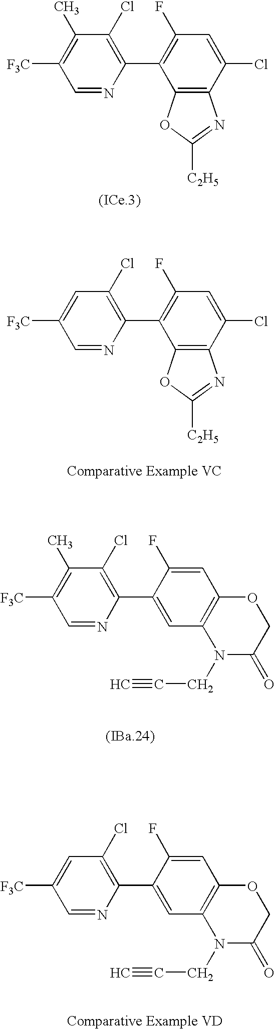

The post-emergence herbicidal action found is compiled in Tables 8 and 9.

TABLE 8
Application

| rate | IAa.3 | | VA | | IAa.10 | | VB | |
|---|---|---|---|---|---|---|---|---|
| [g/ha a.s.] | 31.3 | 15.6 | 31.3 | 15.6 | 7.8 | 3.9 | 7.8 | 3.9 |

Harmful plant/action

| | | | | | | | | |
|---|---|---|---|---|---|---|---|---|
| ECHCG | 100 | 90 | 80 | 30 | 85 | 70 | 70 | 50 |
| SETFA | 100 | 95 | 80 | 70 | 98 | 98 | 90 | 80 |
| BIDPI | 60 | 60 | 40 | 35 | 100 | 100 | 70 | 40 |
| CHEAL | 100 | 100 | 98 | 90 | 100 | 100 | 100 | 100 |

TABLE 9

| Application rate | ICe.3 | | VC | | IBa.24 | | VD | |
|---|---|---|---|---|---|---|---|---|
| [g/ha a.s.] | 7.8 | 3.9 | 7.8 | 3.9 | 3.9 | 1.9 | 3.9 | 1.9 |

Harmful plant/action

| | | | | | | | | |
|---|---|---|---|---|---|---|---|---|
| BRAPL | 90 | 80 | 55 | 40 | — | — | — | — |
| SETFA | 100 | 100 | 95 | 90 | 100 | 70 | 90 | 60 |
| BIDPI | 100 | 100 | 55 | 50 | 100 | 100 | 55 | 50 |

At application rates of 31.3 and 15.6 g of a.s./ha, compound No. IAa.3, applied post-emergence, showed a considerably better action against the harmful plants ECHCG, SETFA, BIDPI and CHEAL than Comparative Example VA.

At application rates of 7.8 and 3.9 g of a.s./ha, compound No. IAa10, applied post-emergence, showed a considerably better action against the harmful plants ECHCG, SETFA and BIDPI than Comparative Example VB.

At application rates of 7.8 and 3.9 g of a.s./ha, compound No. ICe.3, applied post-emergence, showed a considerably better action against the harmful plants BRAPL, SETFA and BIDPI than Comparative Example VC.

At application rates of 3.9 and 1.9 g of a.s./ha, compound No. IBa.24, applied post-emergence, showed a considerably better action against the harmful plants SETFA and BIDPI than Comparative Example VD.

### Use Examples (Desiccant/Defoliant Activity)

The test plants used were young cotton plants with 4 leaves (without cotyledons) which had been grown under greenhouse conditions (relative atmospheric humidity 50 to 70%; day/night temperature 27/20° C.).

The young cotton plants were subjected to leaf treatment to runoff point with aqueous preparations of the active ingredient (with addition of 0.15% by weight of the fatty alcohol alkoxide Plurafac® LF 700, based on the spray mixture). The amount of water applied corresponded to 1000 1/ha (converted). After 13 days, the number of shed leaves and the degree of defoliation were determined in %.

No leaves were shed in the case of the untreated control plants.

We claim:

1. A 2-aryl-5-trifluoromethylpyridine of the formula I 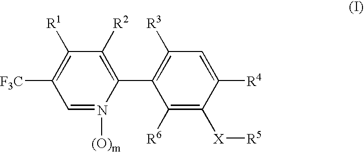
in which the variables m, R¹, R², R³, R⁴, R⁵, R⁶ and X have the following meanings:
m is 0 or 1,
X is a chemical bond, a methylene, 1,2-ethylene, propane-1,3-diyl, ethene-1,2-diyl or ethyne-1,2-diyl chain, or an oxymethylene or thiamethylene chain bonded to the phenyl ring via the hetero atom, it being possible for all chains to be unsubstituted or to have attached to them one or two substituents, in each case selected from the group consisting of cyano, carboxyl, halogen, C₁-C₄-alkyl, C₁-C₄-haloalkyl, C₁-C₄-alkoxy, (C₁-C₄-alkoxy)carbonyl, di(C₁-C₄-alkyl)amino and phenyl;
R¹ is NH₂ or CH₃;
R² is halogen;
R³ is hydrogen or halogen;
R⁴ is halogen, cyano, OH, C₁-C₄-alkoxy or C₁-C₄-alkoxycarbonyl-C₁-C₄-alkoxy;
R⁵ is hydrogen, nitro, cyano, halogen, halosulfonyl, N₃, —O—Y—R⁷, —O—CO—Y—R⁷, —N (Y—R⁷)(Z-R⁸), —N(Y—R⁷)—SO₂-Z-R⁸, —N(SO₂—Y—R⁷)(SO₂-Z-R⁸), —N(Y—R⁷)—CO-Z-R⁸, —N(Y—R⁷)(O-Z-R⁸), —S—Y—R⁷, —SO—Y—R⁷, —SO₂—Y—R⁷, —SO₂—O—Y—R⁷, —SO₂—N(Y—R⁷)(Z-R⁸), —CO—Y—R⁷, —C(═NOR⁹)—Y—R⁷, —C(═NOR⁹)—O—Y—R⁷, —CO—O—Y—R⁷, —CO—S—Y—R⁷, —CO—N(Y—R⁷)(Z-R⁸), —CO—N(Y—R⁷)(O-Z-R⁸) or —PO(O—Y—R⁷)₂;
R⁶ is hydrogen; or
R⁴ and X—R⁵ or X—R⁵ and R⁶ are a 3- or 4-membered chain whose chain members, in addition to carbon, can have 1, 2 or 3 hetero atoms selected from among nitrogen, oxygen and sulfur atoms, which hetero atoms can be unsubstituted or can have attached to them, in turn, one, two or three substituents, and whose members can also encompass one or two nonadjacent carbonyl, thiocarbonyl or sulfonyl groups,
Y, Z independently of one another are:
a chemical bond, a methylene or ethylene group which can be unsubstituted or can have attached to it one or two substituents, in each case selected from the group consisting of carboxyl, C₁-C₄-alkyl, C₁-C₄-haloalkyl, (C₁-C₄-alkoxy)carbonyl and phenyl;
R⁷, R⁸ independently of one another are:
hydrogen, C₃-C₈-cycloalkyl-C₁-C₄-alkyl, C₁-C₆-alkyl, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, —CH(R¹⁰) (R¹¹), —C(R¹⁰) (R¹¹)—CN, —C(R¹⁰) (R¹¹)-halogen, —C(R¹⁰) (R¹¹)—OR¹², —C(R¹⁰) (R¹¹)—N(R¹²)R¹³, —C(R¹⁰)(R¹¹)—N(R¹²)—OR¹³, —C(R¹⁰)(R¹¹)—SR¹², —C(R¹⁰) (R¹¹)—SO—R¹², —C(R¹⁰) (R¹¹)—SO₂—R¹², —C(R¹⁰)(R¹¹)—SO₂—OR¹², —C(R¹⁰)(R¹¹)—SO₂—N(R¹²)R¹³, —C(R¹⁰)(R¹¹)—CO—R¹², —C(R¹⁰)(R¹¹)—C(═NOR¹⁴)—R¹², —C(R¹⁰)(R¹¹)—CO—OR¹², —C(R¹⁰)(R¹¹)—CO—SR¹², —C(R¹⁰)(R¹¹)—CO—N(R¹²)R¹³, —C(R¹⁰)(R¹¹)—CO—N(R¹²)—OR¹³, —C(R¹⁰) (R¹¹)—PO(OR¹²)₂,
C₃-C₈-cycloalkyl which can contain a carbonyl or thiocarbonyl ring member,
phenyl or 3-, 4-, 5-, 6- or 7-membered heterocyclyl which can contain a carbonyl or thiocarbonyl ring member, it being possible for each cycloalkyl ring, for the phenyl ring and for each heterocyclyl ring to be unsubstituted or to have attached to it one, two, three or four substituents, in each case selected from the group consisting of cyano, nitro, amino, hydroxyl, carboxyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, (C₁-C₄-alkoxy)carbonyl and di (C₁-C₄-alkyl) amino;
R⁹ is hydrogen, C₁-C₆-alkyl, C₁-C₄-alkoxycarbonyl-C₁-C₄-alkyl, C₄-C₈-cycloalkyl-C₁-C₄-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;
where the variables R¹⁰ to R¹⁴ have the following meanings:
R¹⁰, R¹¹ independently of one another are
hydrogen, C₁-C₄-alkyl, C₁-C₄-alkoxy-C₁-C₄-alkyl, C₁-C₄-alkylthio-C₁-C₄-alkyl, (C₁-C₄-alkoxy)carbonyl-C₁-C₄-alkyl or phenyl-C₁-C₄-alkyl, it being possible for the phenyl ring to be unsubstituted or to have attached to it one to three substituents, in each case selected from the group consisting of cyano, nitro, carboxyl, halogen, C₁-C₄-alkyl, C₁-C₄-haloalkyl and (C₁-C₄-alkoxy)carbonyl;
R¹², R¹³ independently of one another are
hydrogen, C₁-C₆-alkyl, C₁-C₆-haloalkyl, C₁-C₄-alkoxy-C₁-C₄-alkyl, C₂-C₆-alkenyl, C₂-C₆-haloalkenyl, C₂-C₆-alkynyl, C₂-C₆-haloalkynyl, C₃-C₈-cycloalkyl, C₃-C₈-cycloalkyl-C₁-C₄-alkyl, phenyl, phenyl-C₁-C₄-alkyl, 3- to 7-membered heterocyclyl or heterocyclyl-C₁-C₄-alkyl, it being possible for each cycloalkyl and each heterocyclyl ring to contain a carbonyl or thiocarbonyl ring member,
and where each cycloalkyl ring, the phenyl ring and each heterocyclyl ring can be unsubstituted or have attached to it one, two, three or four substituents, in each case selected from the group consisting of cyano, nitro, amino, hydroxyl, carboxyl, halogen, C₁-C₄-alkyl, C₁-C₄-haloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, (C₁-C₄-alkoxy)carbonyl and di(C₁-C₄-alkyl)amino;
R¹⁴ is hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₁-C₄-alkoxycarbonyl-C₁-C₄-alkyl, phenyl or phenyl-C₁-C₄-alkyl;
or an agriculturally useful salt of I.

2. A 2-aryl-5-trifluoromethylpyridine as claimed in claim 1, where R² is fluorine or chlorine.

3. A 2-aryl-5-trifluoromethylpyridine as claimed in claim 1 or 2, where R³ is hydrogen, fluorine or chlorine.

4. A 2-aryl-5-trifluoromethylpyridine as claimed in any of the preceding claims, where R⁴ is chlorine or cyano and R⁶ is hydrogen.

5. A 2-aryl-5-trifluoromethylpyridine as claimed in any of the preceding claims, where R¹ is methyl.

6. A 2-aryl-5-trifluoromethylpyridine as claimed in claim 5, where R² is chlorine, R³ is fluorine, R⁴ is chlorine or cyano and R⁶ is hydrogen.

7. A 2-aryl-5-trifluoromethylpyridine as claimed in claim 6, where X is a single bond and R⁵ is selected from among C₃-C₄-alkynyloxy, OCH(R¹⁹)—COOR²⁰, CO—OR²¹ and COO—CH(R²²)COOR²³ where
R¹⁹, R²² independently of one another are hydrogen or C₁-C₄-alkyl,
R²⁰, R²¹, R²³ are C₁-C₄-alkyl, C₃-C₄-alkenyl, C₃-C₄-alkenyl, C₁-C₄-haloalkyl or C₁-C₄-alkoxy-C₁-C₄-alkyl.

8. A 2-aryl-5-trifluoromethylpyridine as claimed in any of claims 1 to 3 or 5, where R⁴ together with —X—R⁵ is a chain of the formulae:
—O—(C(R¹⁵)(R¹⁶))ₙ—CO—N(R¹⁷)— or —S—(C(R¹⁵)(R¹⁶))ₙ—CO—N(R¹⁷)—,
where the nitrogen atom of the chain is attached to the C atom which, in formula I, has the group —X—R⁵ attached to it, in which the variables n, R¹⁵ to R¹⁷ have the following meanings:
n is 0 or 1,
R¹⁵, R¹⁶ independently of one another are
hydrogen, C₁-C₆-alkyl, C₁-C₆-alkoxy, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, phenyl or phenyl-C₁-C₄-alkyl;
R¹⁷ is hydrogen, hydroxyl, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylsulfonyl, C₁-C₄-haloalkylsulfonyl, C₁-C₄-alkylcarbonyl, C₁-C₄-haloalkylcarbonyl, C₁-C₄-alkoxycarbonyl, C₁-C₄-alkoxy-C₁-C₄-alkyl, C₁-C₄-alkoxycarbonyl-C₁-C₄-alkyl, C₁-C₄-alkoxycarbonyl-C₁-C₄-alkoxy, mono- and di(C₁-C₄-alkyl)aminocarbonyl, mono- and di(C₁-C₄-alkyl)aminocarbonyl-C₁-C₄-alkyl, mono- and di(C₁-C₄-alkyl)aminocarbonyl-C₁-C₄-alkoxy, phenyl, phenyl-C₁-C₄-alkyl, C₃-C₈-cycloalkyl, C₃-C₈-cycloalkyl-C₁-C₄-alkyl, 3-, 4-, 5-, 6- or 7-membered heterocyclyl, 3-, 4-, 5-, 6- or 7-membered heterocyclyl-C₁-C₄-alkyl which has one or two ring hetero atoms selected from among oxygen, nitrogen or sulfur.

9. A 2-aryl-5-trifluoromethylpyridine as claimed in claim 8, where R³ is fluorine or hydrogen.

10. A 2-aryl-5-trifluoromethylpyridine as claimed in any of claims 1 to 3 or 5, where R⁶ together with —X—R⁵ is a chain of the formulae —N═C(R¹⁸)—O— and —N═C(R¹¹)—S— in which the nitrogen atom of the chain is bonded to the C atom in the phenyl ring of the formula I which has the group X—R⁵ attached to it and where
R¹⁸ is hydrogen, halogen, cyano, amino, C₁-C₆-alkyl, C₁-C₆-haloalkyl, C₂-C₆-alkenyl, C₂-C₆-haloalkenyl, C₂-C₆-alkynyl, C₁-C₄-alkoxy, C₁-C₄-haloalkoxy, C₃-C₆-alkenyloxy, C₃-C₆-alkynyloxy, C₁-C₄-alkylamino, di(C₁-C₄-alkyl)amino, C₁-C₄-haloalkoxy, C₁-C₄-alkylthio, C₁-C₄-haloalkylthio, C₁-C₄-alkylsulfinyl, C₁-C₄-haloalkylsulfinyl, C₁-C₄-alkylsulfonyl, C₁-C₄-haloalkylsulfonyl, C₁-C₄-alkylcarbonyl, C₁-C₄-haloalkylcarbonyl, C₁-C₄-alkoxy-C₁-C₄-alkyl, C₁-C₄-alkoxycarbonyl, C₁-C₄-alkoxycarbonyl-C₁-C₄-alkyl, C₁-C₄-alkoxycarbonyl-C₁-C₄-alkoxy, C₁-C₄-alkoxycarbonyl-C₁-C₄-alkylthio, di(C₁-C₄-alkyl) aminocarbonyl, di-(C₁-C₄-alkyl) aminocarbonyl-C₁-C₄-alkyl, di(C₁-C₄-alkyl) aminocarbonyl-C₁-C₄-alkoxy, di(C₁-C₄-alkyl) aminocarbonyl-C₁-C₄-alkylthio, C₃-C₈-cycloalkyl, phenyl, phenyl-C₁-C₄-alkyl, C₃-C₈-cycloalkyl-C₁-C₄-alkyl, 3-, 4-, 5-, 6- or 7-membered heterocyclyl which has one or two ring hetero atoms selected from among oxygen, nitrogen or sulfur.

11. A 2-aryl-5-trifluoromethylpyridine as claimed in claim 10, where R³ is fluorine or hydrogen and R⁴ is chlorine or cyano.

12. The use of a 2-aryl-5-trifluoromethylpyridine of the formula I and of its agriculturally useful salts as claimed in claim 1 as herbicides or for the desiccation/defoliation of plants.

13. A composition comprising a herbicidally effective amount of at least one 2-aryl-5-trifluoromethylpyridine of the formula I or of an agriculturally useful salt of I as claimed in claim 1 and at least one inert liquid and/or solid carrier and, if desired, at least one surface-active substance.

14. A composition for the desiccation and/or defoliation of plants comprising such an amount of at least one 2-aryl-5-trifluoromethylpyridine of the formula I or of an agriculturally useful salt of I as claimed in claim 1 and at least one inert liquid and/or solid carrier and, if desired, at least one surface-active agent that it has a desiccant and/or defoliant action.

15. A method of controlling undesired vegetation, which comprises allowing a herbicidally active amount of at least one 2-aryl-5-trifluoromethylpyridine of the formula I or of an agriculturally useful salt of I as claimed in claim 1 to act on plants, their environment or on seed.

16. A method for the desiccation and/or defoliation of plants, which comprises allowing such an amount of at least one 2-aryl-5-trifluoromethylpyridine of the formula I or of an agriculturally useful salt of I as claimed in claim 1 to act on plants that it has a desiccant and/or defoliant action.

17. A method as claimed in claim 16, wherein cotton is treated.

18. A pyridine compound of the formula II 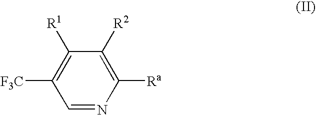
in which the variables R¹ and R² have the meanings given in claim 1 and
R^{a} is halogen, OH, benzyloxy, C₁-C₄-alkoxy or is S(O)ₖphenyl where k is 0, 1 or 2.

19. A boronic acid compound of the formula IIIa 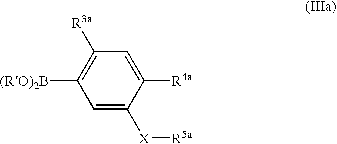
in which X is a single bond and the variables R′, R^{3a}, R^{4a} and R^{5a} have the following meanings:
R′ is hydrogen or C₁-C₁₀-alkyl or two radicals R′ together form a chain of the formula —CH₂—CH₂— or —CH₂—CH₂—CH₂—,
R^{3a} is hydrogen or halogen;
R^{4a} is halogen or C₁-C₄-alkoxy;
R^{5a} is hydrogen, cyano, halogen, —O—Y—R^{7a}, —O—CO—Y—R⁷, —S—Y—R^{7a}, —CO—O—Y—R⁷ or —PO(O—Y—R^{7a})₂; where R^{7a} is a group —C(R¹⁰)(R¹¹)—CO—OR¹² and Y, R⁷, R¹⁰, R¹¹ and R¹² have the meanings given in claim 1;
or R^{4a} is CN and R^{5a} have the following meanings:
R^{5a} is cyano, halogen, —O—Y—R⁷, —O—CO—Y—R⁷, —S—Y—R⁷, —CO—O—Y—R⁷ or —PO(O—Y—R⁷) ₂; where Y and R⁷ have the meanings given in claim 1.
